(19) United States
(12) Patent Application Publication
(10) Pub. No.: US 2007/0238094 A1
(43) Pub. Date: Oct. 11, 2007

(54) Diagnosis, prognosis and monitoring of disease progression of systemic lupus erythematosus through blood leukocyte microarray analysis

(75) Inventors: Damien Chaussabel, Richardson, TX (US); Maria Virginia Pascual, Dallas, TX (US); Jacques F. Banchereau, Dallas, TX (US)

Correspondence Address: CHALKER FLORES, LLP, 2711 LBJ FRWY, Suite 1036, DALLAS, TX, 75234, US

(73) Assignee: Baylor Research Institute, Dallas, TX (US)

(21) Appl. No.: 11/446,825

(22) Filed: Jun. 5, 2006

Related U.S. Application Data
(60) Provisional application No. 60/748,884, filed on Dec. 9, 2005.

(51) Int. Cl. C12Q 1/70; C12Q 1/68; C12M 3/00

(52) U.S. Cl. 435/5; 435/6; 435/287.2

(57) ABSTRACT

The present invention includes compositions, systems, arrays and methods for the early detection and consistent determination of SLE using modular analysis of gene expression data.

### STATEMENT OF FEDERALLY FUNDED RESEARCH

This invention was made with U.S. Government support under National Institutes of Health Contract Nos. R01-01 AR46589, CA78846 and U19 A1057234-02. The government has certain rights in this invention.

### TECHNICAL FIELD OF THE INVENTION

The present invention relates in general to the field of diagnostic for Systemic Lupus Erythematosus, and more particularly, to a system, method and apparatus for the diagnosis, prognosis and monitoring of Systemic Lupus Erythematosus disease progression before, during and after treatment.

### LENGTHY TABLE

The patent application contains a lengthy table section. A copy of the table is available in electronic form from the USPTO web site (http://seqdata.uspto.gov/). An electronic copy of F the table will also be available from the USPTO upon request and payment of the fee set forth in 37 CFR 1.19(b)(3).

LENGTHY TABLES FILED ON CD
The patent application contains a lengthy table section. A copy of the table is available in electronic form from the USPTO web site (http://seqdata.uspto.gov/?pageRequest=docDetail&DocID=US20070238094A1). An electronic copy of the table will also be available from the USPTO upon request and payment of the fee set forth in 37 CFR 1.19(b)(3).

### BACKGROUND OF THE INVENTION

This application claims priority to U.S. Provisional Patent Application Ser. No. 60/748,884, filed Dec. 9, 2005, the entire contents of which are incorporated herein by reference. Without limiting the scope of the invention, its background is described in connection with the diagnosis, prognosis and monitoring of disease progression.

Systemic Lupus Erythematosus (SLE) is an autoimmune disease characterized by dysregulation of innate and adaptive immunity (1-6). The disease course is characterized by recurrent flares which cannot be predicted and worsen the status of the patient. Current treatments are based on non-specific immune suppression, which underscores the need to identify new targets for therapeutic intervention. Studies in mice and humans provide strong evidence that interferon-alpha, a potent anti-viral cytokine, contributes to the SLE immune system abnormalities and may represent one such new target (7-9).

Clinical trials to test new therapeutic agents, however, are hampered by the heterogeneity of SLE clinical manifestations and the lack of reliable markers of disease activity and end organ damage. At least 6 composite measures of SLE global disease activity are available (10-15). These instruments provide metrics to document and quantify disease activity and have been used in clinical trials. Some of the included measures, however, are not easy to obtain. Conversely, given the heterogeneous nature of the clinical disease, not all SLE manifestations are computed in these instruments, making the overall assessment of the patient condition difficult. Hence, there is an important need to develop better systems to assess global disease activity, e.g., to monitor disease progression.

Current methods for determining and following SLE disease-activity and constitutional-symptom variables characterizing the individual's SLE condition include, e.g., SLE Disease activity index (SLEDAI), Systemic Lupus Activity Measurement (SLAM), Patient Visual Analog Scale (Patient VAS), and Krupp Fatigue Severity Score (KFSS). The differences between the values for SLEDAI, KFSS, VAS, and SLAM after initiating evaluation and baseline values for SLEDAI, KFSS, VAS, and SLAM before initiating therapy are determined.

Although SLE preferentially affects women in child bearing years, up to 20% of patients are diagnosed before the age of 18. Presentation, clinical symptoms and immunological findings are similar in pediatric and adult SLE patients. Children, however, tend to have a more severe disease at onset, higher incidence of organ involvement and a more aggressive clinical course than adult patients (16-18). The diagnosis of SLE in children is based upon the same criteria used in adults (19, 20).

The presence of anti-nuclear antibodies (ANA) in serum is a universal finding in SLE. However, up to 5-10% of the normal population displays a positive ANA test at low titer (21). When patients suffering from chronic musculoskeletal pain have positive ANA titers (22) they may be misdiagnosed with SLE and undergo unnecessary tests and lengthy treatments. One such syndrome is fibromyalgia, a condition that affects both adults and children (23).

### SUMMARY OF THE INVENTION

Genomic research is facing significant challenges with the analysis of transcriptional data that are notoriously noisy, difficult to interpret and do not compare well across laboratories and platforms. The present inventors have developed an analytical strategy emphasizing the selection of biologically relevant genes at an early stage of the analysis, which are consolidated into analytical modules that overcome the inconsistencies among microarray platforms. The transcriptional modules developed may be used for the analysis of large gene expression datasets. The results derived from this analysis are easily interpretable and particularly robust, as demonstrated by the high degree of reproducibility observed across commercial microarray platforms.

Applications for this analytical process are illustrated through the mining of a large set of PBMC transcriptional profiles. Twenty-eight transcriptional modules regrouping 4742 genes were identified. Using the present invention is it possible to demonstrate that diseases are uniquely characterized by combinations of transcriptional changes in, e.g., blood leukocytes, measured at the modular level. Indeed, module-level changes in blood leukocytes transcriptional levels constitute the molecular fingerprint of a disease or sample.

This invention has a broad range of applications, e.g., to characterize modular transcriptional components of any biological system (e.g., peripheral blood mononuclear cells (PBMCs), blood cells, fecal cells, peritoneal cells, solid organ biopsies, resected tumors, primary cells, cells lines, cell clones, etc.). Modular PBMC transcriptional data generated through this approach can be used for molecular diagnostic, prognostic, assessment of disease severity, response to drug treatment, drug toxicity, etc. Other data processed using this approach can be employed for instance in mechanistic studies, or screening of drug compounds. In fact, the data analysis strategy and mining algorithm can be implemented in generic gene expression data analysis software and may even be used to discover, develop and test new, disease- or condition-specific modules. The present invention may also be used in conjunction with pharmacogenomics, molecular diagnostic, bioinformatics and the like, where in in-depth expression data may be used to improve the results (e.g., by improving or sub-selecting from within the sample population) that may be obtained during clinical trails.

More particularly, the present invention includes arrays, apparatuses, systems and method for diagnosing a disease or condition by obtaining the transcriptome of a patient; analyzing the transcriptome based on one or more transcriptional modules that are indicative of a disease or condition; and determining the patient's disease or condition based on the presence, absence or level of expression of genes within the transcriptome in the one or more transcriptional modules. The transcriptional modules may be obtained by: iteratively selecting gene expression values for one or more transcriptional modules by: selecting for the module the genes from each cluster that match in every disease or condition; removing the selected genes from the analysis; and repeating the process of gene expression value selection for genes that cluster in a sub-fraction of the diseases or conditions; and iteratively repeating the generation of modules for each clusters until all gene clusters are exhausted.

Examples of clusters selected for use with the present invention include, but are not limited to, expression value clusters, keyword clusters, metabolic clusters, disease clusters, infection clusters, transplantation clusters, signaling clusters, transcriptional clusters, replication clusters, cell-cycle clusters, siRNA clusters, miRNA clusters, mitochondrial clusters, T cell clusters, B cell clusters, cytokine clusters, lymphokine clusters, heat shock clusters and combinations thereof. Examples of diseases or conditions for analysis using the present invention include, e.g., autoimmune disease, a viral infection a bacterial infection, cancer and transplant rejection. More particularly, diseases for analysis may be selected from one or more of the following conditions: systemic onset juvenile idiopathic arthritis, systemic lupus erythematosus, type I diabetes, liver transplant recipients, melanoma patients, and patients bacterial infections such as Escherichia coli, Staphylococcus aureus, viral infections such as influenza A, and combinations thereof. Specific array may even be made that detect specific diseases or conditions associated with a bioterror agent.

Cells that may be analyzed using the present invention, include, e.g., peripheral blood mononuclear cells (PBMCs), blood cells, fetal cells, peritoneal cells, solid organ biopsies, resected tumors, primary cells, cells lines, cell clones and combinations thereof. The analytical tools described herein may be used to analyze the expression of genes within certain modules in a variety of organisms, e.g., mouse, rat, dog, bovine, ovine, equine, zebrafish, etc. The cells may be single cells, a collection of cells, tissue, cell culture, cells in bodily fluid, e.g., blood. Cells may be obtained from a tissue biopsy, one or more sorted cell populations, cell culture, cell clones, transformed cells, biopies or a single cell. The types of cells may be, e.g., brain, liver, heart, kidney, lung, spleen, retina, bone, neural, lymph node, endocrine gland, reproductive organ, blood, nerve, vascular tissue, and olfactory epithelium cells. After cells are isolated, these mRNA from these cells is obtained and individual gene expression level analysis is performed using, e.g., a probe array, PCR, quantitative PCR, bead-based assays and combinations thereof. The individual gene expression level analysis may even be performed using hybridization of nucleic acids on a solid support using cDNA made from mRNA collected from the cells as a template for reverse transcriptase.

The present invention includes a system and a method to analyze samples for the prognosis, diagnosis and monitoring of disease progression of Systemic Lupus Erythematosus (SLE) using multivariate gene expression analysis. The gene expression differences that remain can be attributed with a high degree of confidence to the unmatched variation. The gene expression differences thus identified can be used, for example, to diagnose disease, identify physiological state, design drugs, and monitor therapies.

In one embodiment, the present invention includes a method of identifying a human subject predisposed to SLE by determining the expression level of one or more biomarker that form part of a gene module, as described herein, such as the genes within the modules as described herein below:

| | Total number | | | | |
|---|---|---|---|---|---|
| | of transcripts | Number of transcripts | | % transcripts | |
| | per Module | Overexpressed | Underexpressed | Overexpressed | Underexpressed |
| M1.1 | 76 | 34 | 0 | | 0 |
| M1.7 | 129 | 0 | 101 | 0 | [symbol] |
| M2.1 | 95 | 2 | 20 | 2 | [symbol] |
| M2.2 | 49 | 16 | 0 | | 0 |
| M2.3 | 148 | 39 | 4 | | 3 |
| M2.4 | 133 | 0 | 102 | 0 | [symbol] |
| M2.5 | 315 | 3 | 86 | 1 | [symbol] |
| M2.6 | 165 | 38 | 3 | | 2 |
| M2.7 | 71 | 1 | 22 | 1 | [symbol] |
| M2.8 | 141 | 0 | 59 | 0 | [symbol] |
| M3.1 | 122 | 111 | 0 | | 0 |

While the following modules are listed by a letter and number for use in this example, the module includes one or more of the listed genes (and their complements or equivalents) that form the modules listed as: M1.7, M2.2; M2.7; and 3.1. As such, the limitation in the module is one or more of the listed genes, e.g., 1, 2, 3, 4, 5, 6, 7, 8, 9, 10, 15, 20, 25, 30, 50, 75, 100 or more of the following genes that are separated into the following modules that may be uses to analyze a transcriptome for the expression of one or more genes that are then processed into one or more expression vectors, that is a composite of the expression levels (and changes thereto) in a patient suspected of a certain autoinflammatory, autoimmune, or other disease (genetic or acquired) for diagnosis, prognosis and even disease treatment and monitoring, including:

Module M1.7 includes one or more of the following genes or gene fragments: UniGene ID; Hs.406683; Hs.514581; Hs.546356; Hs.374553; Hs.448226; Hs.381172; Hs.534255; Hs.406620; Hs.534255; Hs.410817; Hs.136905; Hs.546394; Hs.419463; Hs.5308; Hs.514581; Hs.387804; Hs.546286; Hs.300141; Hs.356366; Hs.433427; Hs.533624; Hs.546356; Hs.370504; Hs.433701; Hs.153177; Hs.150580; Hs.514581; Hs.356794; Hs.419463; Hs.433427; Hs.469473; Hs.380953; Hs.410817; Hs.421257; Hs.408054; Hs.433529; Hs.458476; Hs.439552; Hs.156367; Hs.546291; Hs.546290; Hs.514581; Hs.144835; Hs.439552; Hs.356502; Hs.397609; Hs.446628; Hs.546356; Hs.265174; Hs.425125; Hs.374596; Hs.381126; Hs.381061; Hs.406620; Hs.533977; Hs.447600; Hs.148340; Hs.421907; Hs.448226; Hs.410817; Hs.119598; Hs.433427; Hs.410817; Hs.8102; Hs.446628; Hs.356572; Hs.381123; Hs.515329; Hs.408054; Hs.483877; Hs.386384; Hs.337766; Hs.408073; Hs.546289; Hs.374596; Hs.512199; Hs.119598; Hs.499839; Hs.446588; Hs.356572; Hs.397609; Hs.356572; Hs.144835; Hs.515329; Hs.534833; Hs.374588; Hs.144835; Hs.80545; Hs.546356; Hs.400295; Hs.119598; Hs.408073; Hs.412370; Hs.401929; Hs.425125; Hs.374588; Hs.374588; Hs.356366; Hs.186350; and/or Hs.186350; and;

M2.2 includes one or more of the following genes or gene fragments: UniGene ID; Hs.513711; Hs.375108; Hs.176626; Hs.2962; Hs.41; Hs.99863; Hs.530049; Hs.51120; Hs.480042; Hs.36977; Hs.294176; Hs.529019; Hs.2582; Hs.550853; Hs.529517; and/or Hs.204238; and;

M2.4 includes one or more of the following genes or gene fragments: Hs.518827; Hs.8102; Hs.190968; Hs.508266; Hs.523913; Hs.437594; Hs.515598; Hs.54780; Hs.534384; Hs.527105; Hs.522885; Hs.462341; Hs.127610; Hs.408018; Hs.381219; Hs.6917; Hs.109798; Hs.497581; Hs.369728; Hs.432485; Hs.314359; Hs.409140; Hs.529798; Hs.477028; Hs.107003; Hs.528668; Hs.314359; Hs.6917; Hs.333120; Hs.500822; Hs.131255; Hs.469925; Hs.410817; Hs.277517; Hs.529631; Hs.367900; Hs.408054; Hs.467284; Hs.111099; Hs.378103; Hs.108332; Hs.397609; Hs.80545; Hs.529631; Hs.472558; Hs.519452; Hs.516023; Hs.438429; Hs.515472; Hs.512675; Hs.438429; Hs.314359; Hs.75056; Hs.482526; Hs.333388; Hs.483305; Hs.515329; Hs.288856; Hs.546288; Hs.483305; Hs.534346; Hs.528435; Hs.381219; Hs.469925; Hs.172791; Hs.190968; Hs.182825; Hs.492599; Hs.406620; Hs.549130; Hs.532359; Hs.534346; Hs.421257; Hs.511831; Hs.380920; Hs.311640; Hs.546356; Hs.119598; Hs.405590; Hs.178551; Hs.499839; Hs.148340; Hs.483305; Hs.505735; Hs.381219; Hs.299002; Hs.532359; Hs.5662; Hs.515329; Hs.408073; Hs.515070; Hs.448226; Hs.515329; Hs.511582; Hs.421608; Hs.186350; Hs.529798; and/or Hs.294094; and;

M2.8 includes one or more of the following genes or gene fragments: Hs.397891; Hs.438801; Hs.125036; Hs.210891; Hs.220629; Hs.376208; Hs.316931; Hs.196981; Hs.271272; Hs.397891; Hs.7946; Hs.505326; Hs.369581; Hs.58685; Hs.7236; Hs.17109; Hs.49143; Hs.505806; Hs.60339; Hs.13262; Hs.22380; Hs.233044; Hs.133397; Hs.445489; Hs.60339; Hs.428214; Hs.431498; Hs.533994; Hs.533994; Hs.498317; Hs.533994; Hs.517717; Hs.173135; Hs.522679; Hs.446149; Hs.525700; Hs.519580; Hs.481704; Hs.379414; Hs.125036; Hs.440776; Hs.475602; Hs.173135; Hs.481704; Hs.167087; Hs.142023; Hs.524134; Hs.98309; Hs.433700; Hs.480837; Hs.5019; Hs.525700; Hs.94229; Hs.446149; Hs.502710;

M3.1 includes one or more of the following genes or gene fragments: Hs.276925; Hs.98259; Hs.478275; Hs.273330; Hs.175120; Hs.190622; Hs.175120; Hs.415534; Hs.62661; Hs.344812; Hs.145150; Hs.5148; Hs.302123; Hs.65641; Hs.62661; Hs.86724; Hs.120323; Hs.370515; Hs.291000; Hs.62661; Hs.118110; Hs.131431; Hs.464419; Hs.65641; Hs.145150; Hs.415534; Hs.54483; Hs.520102; Hs.414579; Hs.190622; Hs.374950; Hs.478275; Hs.369039; Hs.229988; Hs.458414; Hs.425777; Hs.531314; Hs.352018; Hs.526464; Hs.470943; Hs.514535; Hs.487933; Hs.481143; Hs.217484; Hs.524117; Hs.137007; Hs.458414; Hs.374650; Hs.470943; Hs.50842; Hs.118633; Hs.130759; Hs.384598; Hs.524760; Hs.441975; Hs.530595; Hs.546467; Hs.529317; Hs.175687; Hs.112420; Hs.1706; Hs.523847; Hs.388733; Hs.163173; Hs.470943; Hs.481141; Hs.171426; Hs.174195; Hs.518201; Hs.118633; Hs.489118; Hs.489118; Hs.193842; Hs.551516; Hs.518203; Hs.371794; Hs.529317; Hs.195642; Hs.12341; Hs.414332; Hs.524760; Hs.479264; Hs.501778; Hs.414332; Hs.12646; Hs.518200; Hs.441975; Hs.441975; Hs.437609; Hs.130759; Hs.82316; Hs.518200; Hs.458485; Hs.31869; Hs.166120; Hs.549041; Hs.17518; Hs.546467; Hs.517307; Hs.549041; Hs.528634; Hs.389724; Hs.546523; Hs.82316; Hs.7155; Hs.521903; Hs.26663; Hs.120323; and/or Hs.926.

wherein the biomarker is correlated with a predisposition and/or prognosis to SLE.

The biomarker may include transcriptional regulation genes selected from upregulation and downregulation of these genes. A specific set of one or more gene modules selected from the group consisting of: one or more “MHC/Ribosomal genes” comprising MHC class I molecules: HLA-A,B,C,G,E)+Beta 2-microglobulin (B2M), Ribosomal proteins: RPLs, RPSs; and genes listed for module M1.7 in attached table;

one or more “Neutrophil genes” comprising Lactotransferrin: LTF, defensin: DEAF1, Bacterial Permeability Increasing protein (BPI), Cathelicidin antimicrobial protein (CAMP); and genes listed for module M2.2 in attached table;

one or more “Ribosomal protein genes” comprising RPLs, RPSs, Eukaryotic Translation Elongation factor family members (EEFs), Nucleolar proteins: NPM1, NOAL2, NAP1L1; and genes listed for module M2.4 in attached table;

one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B; and genes listed for module M2.8 in attached table; and

one or more “interferon-inducible genes” comprising antiviral molecules (OAS1/2/3L, GBP1, G1P2, EIF2AK2/PKR, MX1, PML), chemokines (CXCL10/IP-10), signaling molecules (STAT1, STAt2, IRF7, ISGF3G) and genes listed for module M3.1 in attached table;

and are sufficient to distinguish between SLE, Fibromyalgia, a viral infection a bacterial infection, cancer and transplant rejection. In particular, and with reference to the Lengthy Table incorporated herein by reference, the Modules that may be used for the differentiation between SLE and Fibromyalgia may include: M1.1, M1.7, M2.1, M 2.2, M2.3, M2.4, M2.5, M2.6, M2.7, M 2.8 and M 3.1, each of which may include 1, 2, 3, 4, 5, 6, 7, 8, 9, 10, 15, 20 or more genes for analysis.

The biomarkers may be screened by quantitating the mRNA, protein or both mRNA and protein level of the biomarker. When the biomarker is mRNA level, it may be quantitated by a method selected from polymerase chain reaction, real time polymerase chain reaction, reverse transcriptase polymerase chain reaction, hybridization, probe hybridization, and gene expression array. The screening method may also include detection of polymorphisms in the biomarker. Alternatively, the screening step may be accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. For use with the present invention the sample may be any of a number of immune cells, e.g., total blood cells, leukocytes or sub-components thereof.

Another embodiment includes a method for diagnosing Systemic Lupus Erythematosus (SLE) from a tissue sample that includes obtaining a gene expression profile from the tissue sample wherein expression of the two or more of the following genes is measured from M1.1, M 1.7, M2.1, M 2.2, M2.3, M2.4, M2.5, M2.6, M2.7 M 2.8 and/or M 3.1 as compared to a normal control sample. The tissue used for the source of biomarker, e.g., RNA, may be blood or sub-components thereof.

The arrays, methods and systems of the present invention may even be used to select patients for a clinical trial by obtaining the transcriptome of a prospective patient; comparing the transcriptome to one or more transcriptional modules that are indicative of a disease or condition that is to be treated in the clinical trial; and determining the likelihood that a patient is a good candidate for the clinical trial based on the presence, absence or level of one or more genes that are expressed in the patient's transcriptome within one or more transcriptional modules that are correlated with success in a clinical trial. Generally, for each module a vector that correlates with a sum of the proportion of transcripts in a sample may be used, e.g., when each module includes a vector and wherein one or more diseases or conditions is associated with the one or more vectors. Therefore, each module may include a vector that correlates to the expression level of one or more genes within each module.

The present invention also includes arrays, e.g., custom microarrays, bead arrays, liquid suspension arrays, etc., which include nucleic acid probes immobilized on a solid support that includes sufficient probes from one or more modules to provide a sufficient proportion of differentially expressed genes to distinguish between one or more diseases, the probes being selected from the Table below. For example, an array of nucleic acid probes immobilized on a solid support, in which the array includes at least two sets of probe modules selected from M 1.1, M 1.7, M 2.1, M 2.2, M 2.3, M 2.4, M 2.5, M 2.6, M 2.7 M 2.8 and/or M 3.1, wherein the probes in the first probe set have one or more interrogation positions respectively corresponding to one or more diseases. The array may have between 100 and 100,000 probes, and each probe may be, e.g., 9, 15, 20, 30, 40, 50, 75, 100 or more nucleotides long. In certain embodiments, the length of the probe may be thousands if not hundreds of thousands of bases (e.g., a restriction fragment, plasmid, cosmid and the like). When separated into organized probe sets, these may be interrogated together or separately.

The present invention also includes one or more nucleic acid probes immobilized on a solid support to form a module array that includes at least one pair of first and second probe groups, each group having one or more probes as defined by Table 3 (e.g., those listed in the modules listed as M 1.7, M 2.2, M2.4, M 2.8 and M 3.1). The probe groups are selected to provide a composite transcriptional marker (vector) that is consistent across microarray platforms. In fact, the probe groups may even be used to provide a composite transcriptional vector that is consistent across microarray platforms and displayed in a summary for regulatory approval. The skilled artisan will appreciate that using the modules of the present invention it is possible to rapidly develop one or more disease specific arrays that may be used to rapidly diagnose or distinguish between different disease and/or conditions.

A method for determining whether an individual has systemic lupus erythematosus (SLE), by obtaining the transcriptome of a patient, scoring the transcriptome based on one or more transcriptional modules; and determining the patient's disease or condition based on the presence, absence or level of expression of genes within the transcriptome in the one or more transcriptional modules that are indicative of SLE. More particularly, the transcriptional modules are obtained by: iteratively selecting gene expression values for one or more transcriptional modules by: selecting for the module the genes from each cluster that match in every disease or condition; removing the selected genes from the analysis; and repeating the process of gene expression value selection for genes that cluster in a sub-fraction of the diseases or conditions; and iteratively repeating the generation of modules for each clusters until all gene clusters are exhausted. The clusters may be selected from expression value clusters, keyword clusters, metabolic clusters, disease clusters, infection clusters, transplantation clusters, signaling clusters, transcriptional clusters, replication clusters, cell-cycle clusters, siRNA clusters, miRNA clusters, mitochondrial clusters, T cell clusters, B cell clusters, cytokine clusters, lymphokine clusters, heat shock clusters and combinations thereof. The patient may be a human SLE patient and may even be provided with a therapeutically effective amount of a drug selected from the group of: a glucocorticoid, a non-steroidal anti-inflammatory agent and an immunosuppressant.

The present invention also includes a method of diagnosing or monitoring an autoimmune or chronic inflammatory disease in a patient, comprising detecting the expression level of two or more gene modules that include genes selected from: immunoglobulin, neutrophils, interferon, T cells, and ribosomal proteins. The one or more genes may be selected from M 1.7, M 2.2, M2.4, M 2.8 and M 3.1 and the disease is systemic lupus erythematosus (SLE).

In another embodiment, the expression level of the genes or its products are detected by measuring the RNA level expressed by the gene. The method may also include isolating RNA from the patient prior to detecting the RNA level expressed by the gene, wherein the RNA level is detected by PCR and/or by hybridization, e.g., to a complementary oligonucleotide. In certain embodiments, the analysis of gene expression may also use probes that are DNA, RNA, cDNA, PNA, genomic DNA, or synthetic oligonucleotides. Alternatively or in conjunction with the above, the level of expression of the genes from the patient may be detected by measuring protein levels of the gene.

Yet another embodiment of the present invention include a disease analysis tool that includes one or more probes that are part of the transcriptions modules that include one or more genes selected from the group consisting of:

### Transcriptional Modules

one or more MHC/Ribosomal genes comprising MHC class I molecules: HLA-A,B,C,G,E)+Beta 2-microglobulin (B2M), Ribosomal proteins: RPLs, RPSs; & genes listed for module M1.7 in attached table

one or more Neutrophil genes comprising Lactotransferrin: LTF, defensin: DEAF1, Bacterial Permeability Increasing protein (BPI), Cathelicidin antimicrobial protein (CAMP); & genes listed for module M2.2 in attached table and

one or more Ribosomal protein genes comprising RPLs, RPSs, Eukaryotic Translation Elongation factor family members (EEFs), Nucleolar proteins: NPM1, NOAL2, NAP1L1; & genes listed for module M2.4 in attached table and

one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B; & genes listed for module M2.8 in attached table and

one or more interferon-inducible genes comprising antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G) & genes listed for module M3.1 in attached table;

sufficient to distinguish between an autoimmune disease (e.g., SLE), a viral infection a bacterial infection, cancer and transplant rejection.

Another embodiment is a prognostic gene array that is a customized gene array that includes a combination of genes that are representative of one or more transcriptional modules, wherein the transcriptome of a patient that is contacted with the customized gene array is prognostic of SLE. The array may be used to monitor the patient's response to therapy for SLE. The array may also be used to distinguish between an autoimmune disease, a viral infection a bacterial infection, cancer and transplant rejection. For certain direct measurement purposes the array may even be organized into two or more transcriptional modules that may be visually scanned and the extent of expression analyzed optically, e.g., with the naked eye and/or with image processing equipment. For example, the array may be organized into three transcriptional modules with one or more submodules selected from:

| | Number | | |
|---|---|---|---|
| | of probe | | |
| Module I.D. | sets | Keyword selection | Assessment |
| M 1.1 | 76 | Ig, Immunoglobulin, | Plasma cells. Includes genes coding for |
| | | Bone, Marrow, PreB, | Immunoglobulin chains (e.g. IGHM, IGJ, IGLL1, |
| | | IgM, Mu. | IGKC, IGHD) and the plasma cell marker CD38. |
| M 1.2 | 130 | Platelet, Adhesion, | Platelets. Includes genes coding for platelet |
| | | Aggregation, | glycoproteins (ITGA2B, ITGB3, GP6, GP1A/B), and |
| | | Endothelial, Vascular | platelet-derived immune mediators such as PPPB |
| | | | (pro-platelet basic protein) and PF4 (platelet factor 4). |
| M 1.3 | 80 | Immunoreceptor, BCR, | B-cells. Includes genes coding for B-cell surface |
| | | B-cell, IgG | markers (CD72, CD79A/B, CD19, CD22) and other |
| | | | B-cell associated molecules: Early B-cell factor |
| | | | (EBF), B-cell linker (BLNK) and B lymphoid |
| | | | tyrosine kinase (BLK). |
| M 1.4 | 132 | Replication, | Undetermined. This set includes regulators and |
| | | Repression, Repair, | targets of cAMP signaling pathway (JUND, ATF4, |
| | | CREB, Lymphoid, | CREM, PDE4, NR4A2, VIL2), as well as repressors |
| | | TNF-alpha | of TNF-alpha mediated NF-KB activation (CYLD, |
| | | | ASK, TNFAIP3). |
| M 1.5 | 142 | Monocytes, Dendritic, | Myeloid lineage. Includes molecules expressed by |
| | | MHC, Costimulatory, | cells of the myeloid lineage (CD86, CD163, |
| | | TLR4, MYD88 | FCGR2A), some of which being involved in pathogen |
| | | | recognition (CD14, TLR2, MYD88). This set also |
| | | | includes TNF family members (TNFR2, BAFF). |
| M 1.6 | 141 | Zinc, Finger, P53, RAS | Undetermined. This set includes genes coding for |
| | | | signaling molecules, e.g. the zinc finger containing |
| | | | inhibitor of activated STAT (PIAS1 and PIAS2), or |
| | | | the nuclear factor of activated T-cells NFATC3. |
| M 1.7 | 129 | Ribosome, | MHC/Ribosomal proteins. Almost exclusively |
| | | Translational, 40S, 60S, | formed by genes coding MHC class I molecules |
| | | HLA | (HLA-A, B, C, G, E) + Beta 2-microglobulin (B2M) or |
| | | | Ribosomal proteins (RPLs, RPSs). |
| M 1.8 | 154 | Metabolism, | Undetermined. Includes genes encoding metabolic |
| | | Biosynthesis, | enzymes (GLS, NSF1, NAT1) and factors involved in |
| | | Replication, Helicase | DNA replication (PURA, TERF2, EIF2S1). |
| M 2.1 | 95 | NK, Killer, Cytolytic, | Cytotoxic cells. Includes cytotoxic T-cells amd NK- |
| | | CD8, Cell-mediated, T- | cells surface markers (CD8A, CD2, CD160, NKG7, |
| | | cell, CTL, IFN-g | KLRs), cytolytic molecules (granzyme, perforin, |
| | | | granulysin), chemokines (CCL5, XCL1) and |
| | | | CTL/NK-cell associated molecules (CTSW). |
| M 2.2 | 49 | Granulocytes, | Neutrophils. This set includes innate molecules that |
| | | Neutrophils, Defense, | are found in neutrophil granules (Lactotransferrin: |
| | | Myeloid, Marrow | LTF, defensin: DEAF1, Bacterial Permeability |
| | | | Increasing protein: BPI, Cathelicidin antimicrobial |
| | | | protein: CAMP . . . ). |
| M 2.3 | 148 | Erythrocytes, Red, | Erythrocytes. Includes hemoglobin genes (HGBs) |
| | | Anemia, Globin, | and other erythrocyte-associated genes (erythrocytic |
| | | Hemoglobin | alkirin: ANK1, Glycophorin C: GYPC, |
| | | | hydroxymethylbilane synthase: HMBS, erythroid |
| | | | associated factor: ERAF). |
| M 2.4 | 133 | Ribonucleoprotein, | Ribosomal proteins. Including genes encoding |
| | | 60S, nucleolus, | ribosomal proteins (RPLs, RPSs), Eukaryotic |
| | | Assembly, Elongation | Translation Elongation factor family members (EEFs) |
| | | | and Nucleolar proteins (NPM1, NOAL2, NAP1L1). |
| M 2.5 | 315 | Adenoma, Interstitial, | Undetermined. This module includes genes encoding |
| | | Mesenchyme, Dendrite, | immune-related (CD40, CD80, CXCL12, IFNA5, |
| | | Motor | IL4R) as well as cytoskeleton-related molecules |
| | | | (Myosin, Dedicator of Cytokenesis, Syndecan 2, |
| | | | Plexin C1, Distrobrevin). |
| M 2.6 | 165 | Granulocytes, | Myeloid lineage. Includes genes expressed in |
| | | Monocytes, Myeloid, | myeloid lineage cells (IGTB2/CD18, Lymphotoxin |
| | | ERK, Necrosis | beta receptor, Myeloid related proteins 8/14 Formyl |
| | | | peptide receptor 1), such as Monocytes and |
| | | | Neutrophils. |
| M 2.7 | 71 | No keywords extracted. | Undetermined. This module is largely composed of |
| | | | transcripts with no known function. Only 20 genes |
| | | | associated with literature, including a member of the |
| | | | chemokine-like factor superfamily (CKLFSF8). |
| M 2.8 | 141 | Lymphoma, T-cell, | T-cells. Includes T-cell surface markers (CD5, CD6, |
| | | CD4, CD8, TCR, | CD7, CD26, CD28, CD96) and molecules expressed |
| | | Thymus, Lymphoid, | by lymphoid lineage cells (lymphotoxin beta, IL2- |
| | | IL2 | inducible T-cell kinase, TCF7, T-cell differentiation |
| | | | protein mal, GATA3, STAT5B). |
| M 2.9 | 159 | ERK, Transactivation, | Undetermined. Includes genes encoding molecules |
| | | Cytoskeletal, MAPK, | that associate to the cytoskeleton (Actin related |
| | | JNK | protein 2/3, MAPK1, MAP3K1, RAB5A). Also |
| | | | present are T-cell expressed genes (FAS, |
| | | | ITGA4/CD49D, ZNF1A1). |
| M 2.10 | 106 | Myeloid, Macrophage, | Undetermined. Includes genes encoding for Immune- |
| | | Dendritic, | related cell surface molecules (CD36, CD86, LILRB), |
| | | Inflammatory, | cytokines (IL15) and molecules involved in signaling |
| | | Interleukin | pathways (FYB, TICAM2-Toll-like receptor |
| | | | pathway). |
| M 2.11 | 176 | Replication, Repress, | Undetermined. Includes kinases (UHMK1, |
| | | RAS, | CSNK1G1, CDK6, WNK1, TAOK1, CALM2, |
| | | Autophosphorylation, | PRKCI, ITPKB, SRPK2, STK17B, DYRK2, PIK3R1, |
| | | Oncogenic | STK4, CLK4, PKN2) and RAS family members |
| | | | (G3BP, RAB14, RASA2, RAP2A, KRAS). |
| M 3.1 | 122 | ISRE, Influenza, | Interferon-inducible. This set includes interferon- |
| | | Antiviral, IFN-gamma, | inducible genes: antiviral molecules (OAS1/2/3/L, |
| | | IFN-alpha, Interferon | GBP1, G1P2, EIF2AK2/PKR, MX1, PML), |
| | | | chemokines (CXCL10/IP-10), signaling molecules |
| | | | (STAT1, STAt2, IRF7, ISGF3G). |
| M 3.2 | 322 | TGF-beta, TNF, | Inflammation I. Includes genes encoding molecules |
| | | Inflammatory, | involved in inflammatory processes (e.g. IL8, |
| | | Apoptotic, | ICAM1, C5R1, CD44, PLAUR, IL1A, CXCL16), and |
| | | Lipopolysaccharide | regulators of apoptosis (MCL1, FOXO3A, RARA, |
| | | | BCL3/6/2A1, GADD45B). |
| M 3.3 | 276 | Inflammatory, Defense, | Inflammation II. Includes molecules inducing or |
| | | Lysosomal, Oxidative, | inducible by inflammation (IL18, ALOX5, ANPEP, |
| | | LPS | AOAH, HMOX1, SERPINB1), as well as lysosomal |
| | | | enzymes (PPT1, CTSB/S, NEU1, ASAH1, LAMP2, |
| | | | CAST). |
| M 3.4 | 325 | Ligase, Kinase, KIP1, | Undetermined. Includes protein phosphatases |
| | | Ubiquitin, Chaperone | (PPP1R12A, PTPRC, PPP1CB, PPM1B) and |
| | | | phosphoinositide 3-kinase (PI3K) family members |
| | | | (PIK3CA, PIK32A, PIP5K3). |
| M 3.5 | 22 | No keyword extracted | Undetermined. Composed of only a small number of |
| | | | transcripts. Includes hemoglobin genes (HBA1, |
| | | | HBA2, HBB). |
| M 3.6 | 288 | Ribosomal, T-cell, | Undetermined. This set includes mitochondrial |
| | | Beta-catenin | ribosomal proteins (MRPLs, MRPs), mitochondrial |
| | | | elongations factors (GFM1/2), Sortin Nexins |
| | | | (SN1/6/14) as well as lysosomal ATPases |
| | | | (ATP6V1C/D). |
| M 3.7 | 301 | Spliceosome, | Undetermined. Includes genes encoding proteasome |
| | | Methylation, Ubiquitin | subunits (PSMA2/5, PSMB5/8); ubiquitin protein |
| | | | ligases HIP2, STUB1, as well as components of |
| | | | ubiqutin ligase complexes (SUGT1). |
| M 3.8 | 284 | CDC, TCR, CREB, | Undetermined. Includes genes encoding enzymes: |
| | | Glycosylase | aminomethyltransferase, arginyltransferase, |
| | | | asparagines synthetase, diacylglycerol kinase, inositol |
| | | | phosphatases, methyltransferases, helicases . . . |
| M 3.9 | 260 | Chromatin, Checkpoint, | Undetermined. Includes genes encoding kinases |
| | | Replication, | (IBTK, PRKRIR, PRKDC, PRKCI) and phosphatases |
| | | Transactivation | (e.g. PTPLB, PPP2CB/3CB, PTPRC, MTM1, |
| | | | MTMR2). |

wherein probes that bind specifically to one or more of the genes are selected from within the three or more modules and are indicative of systemic lupus erythematosus.

Another embodiment of the present invention includes a method for selecting patients for a clinical trial by obtaining the transcriptome of a prospective patient; comparing the transcriptome to one or more transcriptional modules that are indicative of a disease or condition that is to be treated in the clinical trial; and determining the likelihood that a patient is a good candidate for the clinical trial based on the presence, absence or level of one or more genes that are expressed in the patient's transcriptome within one or more transcriptional modules that are correlated with success in a clinical trial. For use with the method, each module may include a vector that correlates with a sum of the proportion of transcripts in a sample; a vector wherein one or more diseases or conditions are associated with the one or more vectors; a vector that correlates to the expression level of one or more genes within each module and/or a vector that includes modules for the detection, characterization, diagnosis, prognosis and/or monitoring of normal versus SLE patients (or other patients (e.g., fibromyalgia)) selected from:

### Transcriptional Modules

M 1.7 one or more MHC/Ribosomal genes comprising MHC class I molecules: HLA-A,B,C,G,E)+Beta 2-microglobulin (B2M), Ribosomal proteins: RPLs, RPSs;
M 2.2 one or more Neutrophil genes comprising Lactotransferrin: LTF, defensin: DEAF1, Bacterial Permeability Increasing protein (BPI), Cathelicidin antimicrobial protein (CAMP);
M 2.4 one or more Ribosomal protein genes comprising RPLs, RPSs, Eukaryotic Translation Elongation factor family members (EEFs), Nucleolar proteins: NPM1, NOAL2, NAP1L1;
M 2.8 one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B; and
M 3.1 one or more interferon-inducible genes comprising antiviral molecules (OAS1/2/3/L, GBP1, GIP2, EIF2AK2/PKR, MX1, PML), chemokines (CXCL10/IP-10), signaling molecules (STAT1, STAt2, IRF7, ISGF3G).
   and combinations thereof.

Yet another embodiment is an array of nucleic acid probes immobilized on a solid support with sufficient probes from one or more modules to provide a sufficient proportion of differentially expressed genes to distinguish between one or more diseases, the probes being selected from Table 4. Another embodiment is a prognostic gene array that includes a customized gene array that has disposed thereon a combination of probes that are prognostic of SLE and the probes are selected from M 1.7, M 2.2, M2.4, M 2.8 and M 3.1.

### BRIEF DESCRIPTION OF THE DRAWINGS

For a more complete understanding of the features and advantages of the present invention, reference is now made to the detailed description of the invention along with the accompanying figures and in which:

FIGS. 1a to 1c summarize the microarray data analysis strategies schema representing the steps involved in accepted gene-level microarray data analyses (1a), and the proposed modular data analysis strategy (1b). A full size representation of the module extraction algorithm is provided in FIG. 1c. FIG. 1c: Module extraction algorithm. Data are generated in the context of a defined experimental system (e.g., ex vivo PBMCs). Transcriptional profiles are obtained for several experimental groups (e.g., G1-8). For each group, genes are distributed among x clusters (e.g., x=30) based on similarity of expression profiles (using K-means clustering algorithm). The cluster distribution of each gene across the different experimental groups is recorded in a table and distribution patterns are matched. Modules are selected through an iterative process, starting with the largest set of genes distributed among the same cluster across all experimental groups (e.g., found in the same cluster for eight out of eight groups). The selection is expanded from this core reference pattern to include genes with 7/8, 6/8 and 5/8 matches. Once a module has been formed, the genes are withdrawn from the selection pool. The process is then repeated, starting with the second largest group of genes, progressively reducing levels of stringency.

FIGS. 2a to 2d show and summarize an analysis of patient blood leukocyte transcriptional profiles. FIG. 2a is the result of a conventional gene-level analysis representing patterns of expression for differentially expressed transcripts between patients with metastatic melanoma or liver transplant recipients and their respective controls (p<0.001, Mann Whitney U test). Clustering analysis grouped genes based on expression patterns and results are represented by a heatmap (overexpressed transcripts=red, underexpressed=blue; The expression of each gene is normalized to the median expression value of the control group). (FIG. 2b) Module-level analysis: Gene expression levels obtained for patients (“Melanoma” or “Transplant”) and respective healthy volunteer PBMCs were compared (p<0.05, Mann-Whitney U test) in modules M1.2, M1.3, M1.4 and M2.1. Pie charts indicate the proportion of genes that were significantly changed. Graphs represent transcriptional profiles of the genes that were significantly changed, with each line showing levels of expression (y-axis) of a single transcript across multiple conditions (samples, x-axis). The expression of each gene is normalized to the median expression value of the control group. (middle panel) Results obtained for the 28 PBMC transcriptional modules are displayed on a grid. The coordinates are used to indicate module IDs (e.g., M2.8 is row M2, column 8). Spots indicate the proportion of genes that were significantly changed for each module. Red spots: proportion of over-expressed genes (i.e. increased gene activity in patients vs. healthy), Blue spots: proportion of under-expressed genes (i.e. decreased gene activity in patients vs. healthy). (lower panel) Functional interpretation is indicated on a grid by a color code. A more detailed functional description of each module can be found in Supplementary Table 1 (attached as a Lengthy Table and incorporated herein by reference). FIGS. 2c and 2d: Modules form coherent transcriptional and functional units a) Coherence in transcriptional behavior is illustrated in a set of samples obtained from 21 healthy volunteers. These samples were not used in the module selection process. The graphs represent transcriptional profiles, with each line showing levels of expression (y-axis) of a single transcript across multiple conditions (samples, x-axis). Transcriptional profiles of Modules 1.2, 1.7, 2.1 and 2.11 are shown. The expression of each gene is normalized to the median of the measurements obtained for that gene across all samples. b) Term occurrence levels in abstracts were computed for all the genes in M3.1, M1.5, M1.3 and M1.2 associated with at least ten publications (representing more than 26,000 abstracts). Keyword profiles were extracted for each module and a selection was used to generate this figure. Levels of keyword occurrence in abstracts are indicated by color scale, with yellow representing high occurrence. M3.1 (e.g., STAT1, CXCL10, OAS2, MX2) is associated with interferon, M1.5 (e.g., MYD88, CD86, TLR2, LILRB2, CD163) is associated with pathogen recognition molecules/myeloid lineage cells, M1.3 (e.g., CD19, CD22, CD72A, BLNK, PAX5) is associated with B-cells and M1.2 (e.g., ITGA2B, PF4, SELP, GP6) is associated with platelets.

FIGS. 3a to 3c show an analysis of significance patterns. FIG. 3a shows the genes expressed at significantly higher levels in both stage 1V melanoma and liver transplant patients compared to healthy volunteers. P-values were obtained from gene expression profiles generated in other diseases: in patients suffering from SLE, GVHD, or acute infections with influenza virus (Influenza A), E. coli, S. pneumoniae (Strep. Pneumo.) or S. aureus (Staph. aureus). Each of these cohorts was compared to their respective control group (healthy volunteers accrued in the context of these studies). The genes were ranked by hierarchical clustering of p-values generated for all the conditions listed above. P-values are represented according to a color scale: Green=Low p-value/significant, White=High p-value/not significant. Distinct significant patterns are identified: P1=ubiquitous; P2=most specific to melanoma and liver transplant groups. FIG. 3b shows the modular distribution of ubiquitous and specific gene signatures common to melanoma and transplant groups. Distribution of P1 (specific—red) and P2 (ubiquitous—blue) transcripts among 28 PBMC transcriptional modules was determined. For each module the proportion of genes shared with either P1 or P2 is represented on a bar graph. FIG. 3c shows a transcriptional signature of immunosuppression. Transcripts overexpressed most specifically in patients with melanoma and transplant recipients (P1) include repressors of immune responses that inhibit: (1) NF-kB translocation; (2) interleukin-2 production and signaling; (3) MAPK pathways and (4) cell proliferation. Some of these factors are well characterized anti-inflammatory molecules, and others are expressed in anergic T-cells.

FIG. 4 shows a schema representing the selection steps leading to the characterization of disease-specific expression vectors.

FIGS. 5a to 5g show some of the immune transcriptional vectors identified from a pediatric SLE patient population sampled prior to the initiation of therapy. Each line on the radar plot represents a patient profile. In FIG. 5a, the thicker line represents the average normalized expression profile for this group of patients. Profiles were generated using the same set of vectors for PBMC isolated from healthy volunteers (FIG. 5b) and an independent cohort of pediatric SLE patients under treatment (FIG. 5c). Averaged normalized expression profiles for treated (green) and untreated (orange) SLE patients cohorts are plotted in (FIG. 5d). Patient profiles were plotted on the same vectors on the basis of clinical activity (SLEDAI), regardless of treatment. Patients with low disease activity (SLEDAI from 0 to 6) are represented in FIG. 5e, and patients with high disease activity (SLEDAI from 14 to 28) are represented in (FIG. 5f). An additional panel is shown in FIG. 5g that summarized the modular transcriptional changes for treated pediatric SLE patients.

FIGS. 6a to 6c show the immune transcriptional vectors identified from a pediatric SLE patient population sampled prior to the initiation of therapy. Each line on the radar plot represents a patient profile. The thicker line represents the average normalized expression profile for this group of patients. Profiles were generated using this set of vectors for PBMC isolated from adult SLE patients under treatment (FIG. 6a), healthy adults (FIG. 6b), and adult subjects diagnosed with fibromyalgia (FIG. 6c).

FIG. 7 shows the expression profiles of genes composing transcriptional vectors M1.7_{SLE}, M2.2_{SLE}, M2.4_{SLE}, M2.8_{SLE} and M3.1_{SLE} that correlate with a clinical SLE disease activity index (SLEDAI). Graphs represent expression level of individual transcripts forming each of the vectors in 12 healthy individuals and 21 untreated pediatric SLE patients. Average expression values across transcripts forming each vector are shown on the graph in yellow. Correlations between averaged vector expression values and SLEDAI are shown below (Spearman correlation).

FIGS. 8a and 8b are graphs that show the Spearman correlations of the multivariate microarray scores (or “genomic scores”—y axis) obtained using averaged expression values of the genes forming vectors M1.7_{SLE}, M2.2_{SLE}, M2.4_{SLE}, M2.8_{SLE}, M3.1_{SLE}, and SLEDAI (x axis). (a) Scores were obtained for 22 untreated pediatric SLE patients. (b) The same analysis was applied to the scores of 31 pediatric SLE patients receiving different combinations of therapy.

FIGS. 9a and 9b show the SLEDAI scores (blue, right y axis) and microarray scores (red, left y axis) of pediatric patients followed longitudinally over time (x axis) (FIG. 9a). Time elapsed between sampling is indicated in months. FIG. 9b shows the SLEDAI scores (blue, right y axis) and U-scores (red, left y axis) of pediatric patients followed longitudinally over time (x axis). Time elapsed between sampling is indicated in months.

FIG. 10 is a cross-platform comparison using PBMC samples from healthy donors and liver transplant recipient analyzed on two different microarray platforms: Affymetrix U133A&B GeneChips and Illumina Sentrix Human Ref8 BeadChips. The same source of total RNA was used to independently prepare biotin-labeled cRNA targets. Results are shown for transcripts that were found on both platforms. The expression of each gene is normalized to the median of the measurements obtained across all samples. The averaged expression values of the genes forming each transcriptional module are shown for both Affymetrix and Illumina platforms.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless defined otherwise, all technical and scientific terms used herein have the meaning commonly understood by a person skilled in the art to which this invention belongs. The following references provide one of skill with a general definition of many of the terms used in this invention: Singleton, et al., Dictionary Of Microbiology And Molecular Biology (2d ed. 1994); The Cambridge Dictionary Of Science And Technology (Walker ed., 1988); The Glossary Of Genetics, 5th Ed., R. Rieger et al. (eds.), Springer Verlag (1991); and Hale & Marham, The Harper Collins Dictionary Of Biology (1991).

Various biochemical and molecular biology methods are well known in the art. For example, methods of isolation and purification of nucleic acids are described in detail in WO 97/10365, WO 97/27317, Chapter 3 of Laboratory Techniques in Biochemistry and Molecular Biology: Hybridization With Nucleic Acid Probes, Part 1. Theory and Nucleic Acid Preparation, (P. Tijssen, ed.) Elsevier, N.Y. (1993); Chapter 3 of Laboratory Techniques in Biochemistry and Molecular Biology Hybridization With Nucleic Acid Probes, Part 1. Theory and Nucleic Acid Preparation, (P. Tijssen, ed.) Elsevier, N.Y. (1993); and Sambrook et al., Molecular Cloning: A Laboratory Manual, Cold Spring Harbor Press, N.Y., (1989); and Current Protocols in Molecular Biology, (Ausubel, F. M. et al., eds.) John Wiley & Sons, Inc., New York (1987-1999), including supplements such as supplement 46 (April 1999).

Bioinformatics Definitions

As used herein, an “object” refers to any item or information of interest (generally textual, including noun, verb, adjective, adverb, phrase, sentence, symbol, numeric characters, etc.). Therefore, an object is anything that can form a relationship and anything that can be obtained, identified, and/or searched from a source. “Objects” include, but are not limited to, an entity of interest such as gene, protein, disease, phenotype, mechanism, drug, etc. In some aspects, an object may be data, as further described below.

As used herein, a “relationship” refers to the co-occurrence of objects within the same unit (e.g., a phrase, sentence, two or more lines of text, a paragraph, a section of a webpage, a page, a magazine, paper, book, etc.). It may be text, symbols, numbers and combinations, thereof

As used herein, “meta data content” refers to information as to the organization of text in a data source. Meta data can comprise standard metadata such as Dublin Core metadata or can be collection-specific. Examples of metadata formats include, but are not limited to, Machine Readable Catalog (MARC) records used for library catalogs, Resource Description Format (RDF) and the Extensible Markup Language (XML). Meta objects may be generated manually or through automated information extraction algorithms.

As used herein, an “engine” refers to a program that performs a core or essential function for other programs. For example, an engine may be a central program in an operating system or application program that coordinates the overall operation of other programs. The term “engine” may also refer to a program containing an algorithm that can be changed. For example, a knowledge discovery engine may be designed so that its approach to identifying relationships can be changed to reflect new rules of identifying and ranking relationships.

As used herein, “statistical analysis” refers to a technique based on counting the number of occurrences of each term (word, word root, word stem, n-gram, phrase, etc.). In collections unrestricted as to subject, the same phrase used in different contexts may represent different concepts. Statistical analysis of phrase co-occurrence can help to resolve word sense ambiguity. “Syntactic analysis” can be used to further decrease ambiguity by part-of-speech analysis. As used herein, one or more of such analyses are referred to more generally as “lexical analysis.” “Artificial intelligence (AI)” refers to methods by which a non-human device, such as a computer, performs tasks that humans would deem noteworthy or “intelligent.” Examples include identifying pictures, understanding spoken words or written text, and solving problems.

As used herein, the term “database” refers to repositories for raw or compiled data, even if various informational facets can be found within the data fields. A database is typically organized so its contents can be accessed, managed, and updated (e.g., the database is dynamic). The term “database” and “source” are also used interchangeably in the present invention, because primary sources of data and information are databases. However, a “source database” or “source data” refers in general to data, e.g., unstructured text and/or structured data, that are input into the system for identifying objects and determining relationships. A source database may or may not be a relational database. However, a system database usually includes a relational database or some equivalent type of database which stores values relating to relationships between objects.

As used herein, a “system database” and “relational database” are used interchangeably and refer to one or more collections of data organized as a set of tables containing data fitted into predefined categories. For example, a database table may comprise one or more categories defined by columns (e.g. attributes), while rows of the database may contain a unique object for the categories defined by the columns. Thus, an object such as the identity of a gene might have columns for its presence, absence and/or level of expression of the gene. A row of a relational database may also be referred to as a “set” and is generally defined by the values of its columns. A “domain” in the context of a relational database is a range of valid values a field such as a column may include.

As used herein, a “domain of knowledge” refers to an area of study over which the system is operative, for example, all biomedical data. It should be pointed out that there is advantage to combining data from several domains, for example, biomedical data and engineering data, for this diverse data can sometimes link things that cannot be put together for a normal person that is only familiar with one area or research/study (one domain). A “distributed database” refers to a database that may be dispersed or replicated among different points in a network.

Terms such “data” and “information” are often used interchangeably, as are “information” and “knowledge.” As used herein, “data” is the most fundamental unit that is an empirical measurement or set of measurements. Data is compiled to contribute to information, but it is fundamentally independent of it. Information, by contrast, is derived from interests, e.g., data (the unit) may be gathered on ethnicity, gender, height, weight and diet for the purpose of finding variables correlated with risk of cardiovascular disease. However, the same data could be used to develop a formula or to create “information” about dietary preferences, i.e., likelihood that certain products in a supermarket have a higher likelihood of selling.

As used herein, “information” refers to a data set that may include numbers, letters, sets of numbers, sets of letters, or conclusions resulting or derived from a set of data. “Data” is then a measurement or statistic and the fundamental unit of information. “Information” may also include other types of data such as words, symbols, text, such as unstructured free text, code, etc. “Knowledge” is loosely defined as a set of information that gives sufficient understanding of a system to model cause and effect. To extend the previous example, information on demographics, gender and prior purchases may be used to develop a regional marketing strategy for food sales while information on nationality could be used by buyers as a guideline for importation of products. It is important to note that there are no strict boundaries between data, information, and knowledge; the three terms are, at times, considered to be equivalent. In general, data comes from examining, information comes from correlating, and knowledge comes from modeling.

As used herein, “a program” or “computer program” refers generally to a syntactic unit that conforms to the rules of a particular programming language and that is composed of declarations and statements or instructions, divisible into, “code segments” needed to solve or execute a certain function, task, or problem. A programming language is generally an artificial language for expressing programs.

As used herein, a “system” or a “computer system” generally refers to one or more computers, peripheral equipment, and software that perform data processing. A “user” or “system operator” in general includes a person, that uses a computer network accessed through a “user device” (e.g., a computer, a wireless device, etc) for the purpose of data processing and information exchange. A “computer” is generally a functional unit that can perform substantial computations, including numerous arithmetic operations and logic operations without human intervention.

As used herein, “application software” or an “application program” refers generally to software or a program that is specific to the solution of an application problem. An “application problem” is generally a problem submitted by an end user and requiring information processing for its solution.

As used herein, a “natural language” refers to a language whose rules are based on current usage without being specifically prescribed, e.g., English, Spanish or Chinese. As used herein, an “artificial language” refers to a language whose rules are explicitly established prior to its use, e.g., computer-programming languages such as C, C++, Java, BASIC, FORTRAN, or COBOL.

As used herein, “statistical relevance” refers to using one or more of the ranking schemes (O/E ratio, strength, etc.), where a relationship is determined to be statistically relevant if it occurs significantly more frequently than would be expected by random chance.

As used herein, the terms “coordinately regulated genes” or “transcriptional modules” are used interchangeably to refer to grouped, gene expression profiles (e.g., signal values associated with a specific gene sequence) of specific genes. Each transcriptional module correlates two key pieces of data, a literature search portion and actual empirical gene expression value data obtained from a gene microarray. The set of genes that is selected into a transcriptional modules is based on the analysis of gene expression data (module extraction algorithm described above). Additional steps are taught by Chaussabel, D. & Sher, A. Mining microarray expression data by literature profiling. Genome Biol 3, RESEARCH0055 (2002), (http://genomebiology.com/2002/3/10/research/0055) relevant portions incorporated herein by reference and expression data obtained from a disease or condition of interest, e.g., Systemic Lupus erythematosus, arthritis, lymphoma, carcinoma, melanoma, acute infection, autoimmune disorders, autoinflammatory disorders, etc.).

The Table below lists examples of keywords that were used to develop the literature search portion or contribution to the transcription modules. The skilled artisan will recognize that other terms may easily be selected for other conditions, e.g., specific cancers, specific infectious disease, transplantation, etc. For example, genes and signals for those genes associated with T cell activation are described hereinbelow as Module ID “M 2.8” in which certain keywords (e.g., Lymphoma, T-cell, CD4, CD8, TCR, Thymus, Lymphoid, IL2) were used to identify key T-cell associated genes, e.g., T-cell surface markers (CD5, CD6, CD7, CD26, CD28, CD96); molecules expressed by lymphoid lineage cells (lymphotoxin beta, IL2-inducible T-cell kinase, TCF7; and T-cell differentiation protein mal, GATA3, STAT5B). Next, the complete module is developed by correlating data from a patient population for these genes (regardless of platform, presence/absence and/or up or downregulation) to generate the transcriptional module. In some cases, the gene profile does not match (at this time) any particular clustering of genes for these disease conditions and data, however, certain physiological pathways (e.g., cAMP signaling, zinc-finger proteins, cell surface markers, etc.) are found within the “Underdetermined” modules. In fact, the gene expression data set may be used to extract genes that have coordinated expression prior to matching to the keyword search, i.e., either data set may be correlated prior to cross-referencing with the second data set.

TABLE 1
Examples of Transcriptional Modules

| Example | | |
|---|---|---|
| Module | Example Keyword | |
| I.D. | selection | Gene Profile Assessment |
| M 1.1 | Ig, Immunoglobulin, Bone, | Plasma cells. Includes genes coding for Immunoglobulin |
| | Marrow, PreB, IgM, Mu. | chains (e.g. IGHM, IGJ, IGLL1, IGKC, IGHD) and the |
| | | plasma cell marker CD38. |
| M 1.2 | Platelet, Adhesion, | Platelets. Includes genes coding for platelet glycoproteins |
| | Aggregation, Endothelial, | (ITGA2B, ITGB3, GP6, GP1A/B), and platelet-derived |
| | Vascular | immune mediators such as PPPB (pro-platelet basic protein) |
| | | and PF4 (platelet factor 4). |
| M 1.3 | Immunoreceptor, BCR, B- | B-cells. Includes genes coding for B-cell surface markers |
| | cell, IgG | (CD72, CD79A/B, CD19, CD22) and other B-cell |
| | | associated molecules: Early B-cell factor (EBF), B-cell |
| | | linker (BLNK) and B lymphoid tyrosine kinase (BLK). |
| M 1.4 | Replication, Repression, | Undetermined. This set includes regulators and targets of |
| | Repair, CREB, Lymphoid, | cAMP signaling pathway (JUND, ATF4, CREM, PDE4, |
| | TNF-alpha | NR4A2, VIL2), as well as repressors of TNF-alpha |
| | | mediated NF-KB activation (CYLD, ASK, TNFAIP3). |
| M 1.5 | Monocytes, Dendritic, | Myeloid lineage. Includes molecules expressed by cells of |
| | MHC, Costimulatory, | the myeloid lineage (CD86, CD163, FCGR2A), some of |
| | TLR4, MYD88 | which being involved in pathogen recognition (CD14, |
| | | TLR2, MYD88). This set also includes TNF family |
| | | members (TNFR2, BAFF). |
| M 1.6 | Zinc, Finger, P53, RAS | Undetermined. This set includes genes coding for signaling |
| | | molecules, e.g., the zinc finger containing inhibitor of |
| | | activated STAT (PIAS1 and PIAS2), or the nuclear factor of |
| | | activated T-cells NFATC3. |
| M 1.7 | Ribosome, Translational, | MHC/Ribosomal proteins. Almost exclusively formed by |
| | 40S, 60S, HLA | genes coding MHC class I molecules (HLA-A, B, C, G, E) + Beta |
| | | 2-microglobulin (B2M) or Ribosomal proteins (RPLs, |
| | | RPSs). |
| M 1.8 | Metabolism, Biosynthesis, | Undetermined. Includes genes encoding metabolic |
| | Replication, Helicase | enzymes (GLS, NSF1, NAT1) and factors involved in DNA |
| | | replication (PURA, TERF2, EIF2S1). |
| M 2.1 | NK, Killer, Cytolytic, | Cytotoxic cells. Includes cytotoxic T-cells and NK-cells |
| | CD8, Cell-mediated, T- | surface markers (CD8A, CD2, CD160, NKG7, KLRs), |
| | cell, CTL, IFN-g | cytolytic molecules (granzyme, perforin, granulysin), |
| | | chemokines (CCL5, XCL1) and CTL/NK-cell associated |
| | | molecules (CTSW). |
| M 2.2 | Granulocytes, Neutrophils, | Neutrophils. This set includes innate molecules that are |
| | Defense, Myeloid, Marrow | found in neutrophil granules (Lactotransferrin: LTF, |
| | | defensin: DEAF1, Bacterial Permeability Increasing protein: |
| | | BPI, Cathelicidin antimicrobial protein: CAMP). |
| M 2.3 | Erythrocytes, Red, | Erythrocytes. Includes hemoglobin genes (HGBs) and |
| | Anemia, Globin, | other erythrocyte-associated genes (erythrocytic |
| | Hemoglobin | alkirin: ANK1, Glycophorin C: GYPC, hydroxymethylbilane |
| | | synthase: HMBS, erythroid associated factor: ERAF). |
| M 2.4 | Ribonucleoprotein, 60S, | Ribosomal proteins. Including genes encoding ribosomal |
| | nucleolus, Assembly, | proteins (RPLs, RPSs), Eukaryotic Translation Elongation |
| | Elongation | factor family members (EEFs) and Nucleolar proteins |
| | | (NPM1, NOAL2, NAP1L1). |
| M 2.5 | Adenoma, Interstitial, | Undetermined. This module includes genes encoding |
| | Mesenchyme, Dendrite, | immune-related (CD40, CD80, CXCL12, IFNA5, IL4R) as |
| | Motor | well as cytoskeleton-related molecules (Myosin, Dedicator |
| | | of Cytokenesis, Syndecan 2, Plexin C1, Distrobrevin). |
| M 2.6 | Granulocytes, Monocytes, | Myeloid lineage. Related to M 1.5. Includes genes |
| | Myeloid, ERK, Necrosis | expressed in myeloid lineage cells (IGTB2/CD18, |
| | | Lymphotoxin beta receptor, Myeloid related proteins 8/14 |
| | | Formyl peptide receptor 1), such as Monocytes and |
| | | Neutrophils. |
| M 2.7 | No keywords extracted. | Undetermined. This module is largely composed of |
| | | transcripts with no known function. Only 20 genes |
| | | associated with literature, including a member of the |
| | | chemokine-like factor superfamily (CKLFSF8). |
| M 2.8 | Lymphoma, T-cell, CD4, | T-cells. Includes T-cell surface markers (CD5, CD6, CD7, |
| | CD8, TCR, Thymus, | CD26, CD28, CD96) and molecules expressed by lymphoid |
| | Lymphoid, IL2 | lineage cells (lymphotoxin beta, IL2-inducible T-cell kinase, |
| | | TCF7, T-cell differentiation protein mal, GATA3, |
| | | STAT5B). |
| M 2.9 | ERK, Transactivation, | Undetermined. Includes genes encoding molecules that |
| | Cytoskeletal, MAPK, JNK | associate to the cytoskeleton (Actin related protein 2/3, |
| | | MAPK1, MAP3K1, RAB5A). Also present are T-cell |
| | | expressed genes (FAS, ITGA4/CD49D, ZNF1A1). |
| M 2.10 | Myeloid, Macrophage, | Undetermined. Includes genes encoding for Immune- |
| | Dendritic, Inflammatory, | related cell surface molecules (CD36, CD86, LILRB), |
| | Interleukin | cytokines (IL15) and molecules involved in signaling |
| | | pathways (FYB, TICAM2-Toll-like receptor pathway). |
| M 2.11 | Replication, Repress, RAS, | Undetermined. Includes kinases (UHMK1, CSNK1G1, |
| | Autophosphorylation, | CDK6, WNK1, TAOK1, CALM2, PRKCI, ITPKB, SRPK2, |
| | Oncogenic | STK17B, DYRK2, PIK3R1, STK4, CLK4, PKN2) and RAS |
| | | family members (G3BP, RAB14, RASA2, RAP2A, KRAS). |
| M 3.1 | ISRE, Influenza, Antiviral, | Interferon-inducible. This set includes interferon-inducible |
| | IFN-gamma, IFN-alpha, | genes: antiviral molecules (OAS1/2/3/L, GBP1, G1P2, |
| | Interferon | EIF2AK2/PKR, MX1, PML), chemokines (CXCL10/IP-10), |
| | | signaling molecules (STAT1, STAt2, IRF7, ISGF3G). |
| M 3.2 | TGF-beta, TNF, | Inflammation I. Includes genes encoding molecules |
| | Inflammatory, Apoptotic, | involved in inflammatory processes (e.g., IL8, ICAM1, |
| | Lipopolysaccharide | C5R1, CD44, PLAUR, IL1A, CXCL16), and regulators of |
| | | apoptosis (MCL1, FOXO3A, RARA, BCL3/6/2A1, |
| | | GADD45B). |
| M 3.3 | Granulocyte, | Inflammation II. Includes molecules inducing or inducible |
| | Inflammatory, Defense, | by Granulocyte-Macrophage CSF (SPI1, IL18, ALOX5, |
| | Oxidize, Lysosomal | ANPEP), as well as lysosomal enzymes (PPT1, CTSB/S, |
| | | CES1, NEU1, ASAH1, LAMP2, CAST). |
| M 3.4 | No keyword extracted | Undetermined. Includes protein phosphates (PPP1R12A, |
| | | PTPRC, PPP1CB, PPM1B) and phosphoinositide 3-kinase |
| | | (PI3K) family members (PIK3CA, PIK32A, PIP5K3). |
| M 3.5 | No keyword extracted | Undetermined. Composed of only a small number of |
| | | transcripts. Includes hemoglobin genes (HBA1, HBA2, |
| | | HBB). |
| M 3.6 | Complement, Host, | Undetermined. Large set that includes T-cell surface |
| | Oxidative, Cytoskeletal, T- | markers (CD101, CD102, CD103) as well as molecules |
| | cell | ubiquitously expressed among blood leukocytes (CXRCR1: |
| | | fraktalkine receptor, CD47, P-selectin ligand). |
| M 3.7 | Spliceosome, Methylation, | Undetermined. Includes genes encoding proteasome |
| | Ubiquitin, Beta-catenin | subunits (PSMA2/5, PSMB5/8); ubiquitin protein ligases |
| | | HIP2, STUB1, as well as components of ubiqutin ligase |
| | | complexes (SUGT1). |
| M 3.8 | CDC, TCR, CREB, | Undetermined. Includes genes encoding for several |
| | Glycosylase | enzymes: aminomethyltransferase, arginyltransferase, |
| | | asparagines synthetase, diacylglycerol kinase, inositol |
| | | phosphatases, methyltransferases, helicases . . . |
| M 3.9 | Chromatin, Checkpoint, | Undetermined. Includes genes encoding for protein kinases |
| | Replication, | (PRKPIR, PRKDC, PRKCI) and phosphatases (e.g., |
| | Transactivation | PTPLB, PPP1R8/2CB). Also includes RAS oncogene family |
| | | members and the NK cell receptor 2B4 (CD244). |

Biological Definitions

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays”, “gene-chips” or DNA chips that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods. Bead arrays that include 50-mer oligonucleotide probes attached to 3 micrometer beads may be used that are, e.g., lodged into microwells at the surface of a glass slide or are part of a liquid phase suspension arrays (e.g., Luminex or Illumina) that are digital beadarrays in liquid phase and uses “barcoded” glass rods for detection and identification.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the term “biological state” refers to the state of the transcriptome (that is the entire collection of RNA transcripts) of the cellular sample isolated and purified for the analysis of changes in expression. The biological state reflects the physiological state of the cells in the sample by measuring the abundance and/or activity of cellular constituents, characterizing according to morphological phenotype or a combination of the methods for the detection of transcripts.

As used herein, the term “expression profile” refers to the relative abundance of RNA, DNA or protein abundances or activity levels. The expression profile can be a measurement for example of the transcriptional state or the translational state by any number of methods and using any of a number of gene-chips, gene arrays, beads, multiplex PCR, quantitiative PCR, run-on assays, Northern blot analysis, Western blot analysis, protein expression, fluorescence activated cell sorting (FACS), enzyme linked immunosorbent assays (ELISA), chemiluminescence studies, enzymatic assays, proliferation studies or any other method, apparatus and system for the determination and/or analysis of gene expression that are readily commercially available.

As used herein, the term “transcriptional state” of a sample includes the identities and relative abundances of the RNA species, especially mRNAs present in the sample. The entire transcriptional state of a sample, that is the combination of identity and abundance of RNA, is also referred to herein as the transcriptome. Generally, a substantial fraction of all the relative constituents of the entire set of RNA species in the sample are measured.

As used herein, the terms “transcriptional vectors,” “expression vectors,” and “genomic vectors” (used interchangeably) refers to transcriptional expression data that reflects the “proportion of differentially expressed genes.” For example, for each module the proportion of transcripts differentially expressed between at least two groups (e.g., healthy subjects vs patients). This vector is derived from the comparison of two groups of samples. The first analytical step is used for the selection of disease-specific sets of transcripts within each module. Next, there is the “expression level.” The group comparison for a given disease provides the list of differentially expressed transcripts for each module. It was found that different diseases yield different subsets of modular transcripts. With this expression level it is then possible to calculate vectors for each module(s) for a single sample by averaging expression values of disease-specific subsets of genes identified as being differentially expressed. This approach permits the generation of maps of modular expression vectors for a single sample, e.g., those described in the module maps disclosed herein. These vector module maps represent an averaged expression level for each module (instead of a proportion of differentially expressed genes) that can be derived for each sample. These composite “expression vectors” are formed through successive rounds of selection: 1) of the modules that were significantly changed across study groups and 2) of the genes within these modules which are significantly changed across study groups. Expression levels are subsequently derived by averaging the values obtained for the subset of transcripts forming each vector. Patient profiles can then be represented by plotting expression levels obtained for each of these vectors on a graph (e.g. on a radar plot). Therefore a set of vectors results from two round of selection, first at the module level, and then at the gene level. Vector expression values are composite by construction as they derive from the average expression values of the transcript forming the vector.

Using the present invention it is possible to identify and distinguish diseases not only at the module-level, but also at the gene-level; i.e., two diseases can have the same vector (identical proportion of differentially expressed transcripts, identical “polarity”), but the gene composition of the expression vector can still be disease-specific. This disease-specific customization permits the user to optimize the performance of a given set of markers by increasing its specificity.

Using modules as a foundation grounds expression vectors to coherent functional and transcriptional units containing minimized amounts of noise. Furthermore, the present invention takes advantage of composite transcriptional markers. As used herein, the term “composite transcriptional markers” refers to the average expression values of multiple genes (subsets of modules) as compared to using individual genes as markers (and the composition of these markers can be disease-specific). The composite transcriptional markers approach is unique because the user can develop multivariate microarray scores to assess disease severity in patients with, e.g., SLE, or to derive expression vectors disclosed herein. The fact that expression vectors are composite (i.e. formed by a combination of transcripts) further contributes to the stability of these markers. Most importantly, it has been found that using the composite modular transcriptional markers of the present invention the results found herein are reproducible across microarray platform, thereby providing greater reliability for regulatory approval. Indeed, vector expression values proved remarkably robust, as indicated by the excellent reproducibility obtained across microarray platforms; as well as the validation results obtained in an independent set of pediatric lupus patients. These results are of importance since improving the reliability of microarray data is a prerequisite for the widespread use of this technology in clinical practice (see, e.g., FDA MAQC program, which aims at establishing reproducibility across array platforms).

Gene expression monitoring systems for use with the present invention may include customized gene arrays with a limited and/or basic number of genes that are specific and/or customized for the one or more target diseases. Unlike the general, pan-genome arrays that are in customary use, the present invention provides for not only the use of these general pan-arrays for retrospective gene and genome analysis without the need to use a specific platform, but more importantly, it provides for the development of customized arrays that provide an optimal gene set for analysis without the need for the thousands of other, non-relevant genes. One distinct advantage of the optimized arrays and modules of the present invention over the existing art is a reduction in the financial costs (e.g., cost per assay, materials, equipment, time, personnel, training, etc.), and more importantly, the environmental cost of manufacturing pan-arrays where the vast majority of the data is irrelevant. The modules of the present invention allow for the first time the design of simple, custom arrays that provide optimal data with the least number of probes while maximizing the signal to noise ratio. By eliminating the total number of genes for analysis, it is possible to, e.g., eliminate the need to manufacture thousands of expensive platinum masks for photolithography during the manufacture of pan-genetic chips that provide vast amounts of irrelevant data. Using the present invention it is possible to completely avoid the need for microarrays if the limited probe set(s) of the present invention are used with, e.g., digital optical chemistry arrays, ball bead arrays, beads (e.g., Luminex), multiplex PCR, quantitiative PCR, run-on assays, Northern blot analysis, or even, for protein analysis, e.g., Western blot analysis, 2-D and 3-D gel protein expression, MALDI, MALDI-TOF, fluorescence activated cell sorting (FACS) (cell surface or intracellular), enzyme linked immunosorbent assays (ELISA), chemiluminescence studies, enzymatic assays, proliferation studies or any other method, apparatus and system for the determination and/or analysis of gene expression that are readily commercially available.

The “molecular fingerprinting system” of the present invention may be used to facilitate and conduct a comparative analysis of expression in different cells or tissues, different subpopulations of the same cells or tissues, different physiological states of the same cells or tissue, different developmental stages of the same cells or tissue, or different cell populations of the same tissue against other diseases and/or normal cell controls. In some cases, the normal or wild-type expression data may be from samples analyzed at or about the same time or it may be expression data obtained or culled from existing gene array expression databases, e.g., public databases such as the NCBI Gene Expression Omnibus database.

As used herein, the term “differentially expressed” refers to the measurement of a cellular constituent (e.g., nucleic acid, protein, enzymatic activity and the like) that varies in two or more samples, e.g., between a disease sample and a normal sample. The cellular constituent may be on or off (present or absent), upregulated relative to a reference or down-regulated relative to the reference. For use with gene-chips or gene-arrays, differential gene expression of nucleic acids, e.g., mRNA or other RNAs (miRNA, siRNA, hnRNA, rRNA, tRNA, etc.) may be used to distinguish between cell types or nucleic acids. Most commonly, the measurement of the transcriptional state of a cell is accomplished by quantitative reverse transcriptase (RT) and/or quantitative reverse transcriptase-polymerase chain reaction (RT-PCR), genomic expression analysis, post-translational analysis, modifications to genomic DNA, translocations, in situ hybridization and the like.

For some disease states it is possible to identify cellular or morphological differences, especially at early levels of the disease state. The present invention avoids the need to identify those specific mutations or one or more genes by looking at modules of genes of the cells themselves or, more importantly, of the cellular RNA expression of genes from immune effector cells that are acting within their regular physiologic context, that is, during immune activation, immune tolerance or even immune anergy. While a genetic mutation may result in a dramatic change in the expression levels of a group of genes, biological systems often compensate for changes by altering the expression of other genes. As a result of these internal compensation responses, many perturbations may have minimal effects on observable phenotypes of the system but, profound effects to the composition of cellular constituents. Likewise, the actual copies of a gene transcript may not increase or decrease, however, the longevity or half-life of the transcript may be affected leading to greatly increases protein production. The present invention eliminates the need of detecting the actual message by, in one embodiment, looking at effector cells (e.g., leukocytes, lymphocytes and/or sub-populations thereof) rather than single messages and/or mutations.

The skilled artisan will appreciate readily that samples may be obtained from a variety of sources including, e.g., single cells, a collection of cells, tissue, cell culture and the like. In certain cases, it may even be possible to isolate sufficient RNA from cells found in, e.g., urine, blood, saliva, tissue or biopsy samples and the like. In certain circumstances, enough cells and/or RNA may be obtained from: mucosal secretion, feces, tears, blood plasma, peritoneal fluid, interstitial fluid, intradural, cerebrospinal fluid, sweat or other bodily fluids. The nucleic acid source, e.g., from tissue or cell sources, may include a tissue biopsy sample, one or more sorted cell populations, cell culture, cell clones, transformed cells, biopies or a single cell. The tissue source may include, e.g., brain, liver, heart, kidney, lung, spleen, retina, bone, neural, lymph node, endocrine gland, reproductive organ, blood, nerve, vascular tissue, and olfactory epithelium.

The present invention includes the following basic components, which may be used alone or in combination, namely, one or more data mining algorithms; one or more module-level analytical processes; the characterization of blood leukocyte transcriptional modules; the use of aggregated modular data in multivariate analyses for the molecular diagnostic/prognostic of human diseases; and/or visualization of module-level data and results. Using the present invention it is also possible to develop and analyze composite transcriptional markers, which may be further aggregated into a single multivariate score.

The present inventors have recognized that current microarray-based research is facing significant challenges with the analysis of data that are notoriously “noisy,” that is, data that is difficult to interpret and does not compare well across laboratories and platforms. A widely accepted approach for the analysis of microarray data begins with the identification of subsets of genes differentially expressed between study groups. Next, the users try subsequently to “make sense” out of resulting gene lists using pattern discovery algorithms and existing scientific knowledge.

Rather than deal with the great variability across platforms, the present inventors have developed a strategy that emphasized the selection of biologically relevant genes at an early stage of the analysis. Briefly, the method includes the identification of the transcriptional components characterizing a given biological system for which an improved data mining algorithm was developed to analyze and extract groups of coordinately expressed genes, or transcriptional modules, from large collections of data.

The biomarker discovery strategy described herein is particularly well adapted for the exploitation of microarray data acquired on a global scale. Starting from ˜44,000 transcripts a set of 28 modules was defined that are composed of nearly 5000 transcripts. Sets of disease-specific composite expression vectors were then derived. Vector expression values (expression vectors) proved remarkably robust, as indicated by the excellent reproducibility obtained across microarray platforms. This finding is notable, since improving the reliability of microarray data is a prerequisite for the widespread use of this technology in clinical practice. Finally, expression vectors can in turn be combined to obtain unique multivariate scores, therefore delivering results in a form that is compatible with mainstream clinical practice. Interestingly, multivariate scores recapitulate global patterns of change rather than changes in individual markers. The development of such “global biomarkers” can be used for both diagnostic and pharmacogenomics fields.

In one example, twenty-eight transcriptional modules regrouping 4742 probe sets were obtained from 239 blood leukocyte transcriptional profiles. Functional convergence among genes forming these modules was demonstrated through literature profiling. The second step consisted of studying perturbations of transcriptional systems on a modular basis. To illustrate this concept, leukocyte transcriptional profiles obtained from healthy volunteers and patients were obtained, compared and analyzed. Further validation of this gene fingerprinting strategy was obtained through the analysis of a published microarray dataset. Remarkably, the modular transcriptional apparatus, system and methods of the present invention using pre-existing data showed a high degree of reproducibility across two commercial microarray platforms.

The present invention includes the implementation of a widely applicable, two-step microarray data mining strategy designed for the modular analysis of transcriptional systems. This novel approach was used to characterize transcriptional signatures of blood leukocytes, which constitutes the most accessible source of clinically relevant information.

As demonstrated herein, it is possible to determine, differential and/or distinguish between two disease based on two vectors even if the vector is identical (+/+) for two diseases—e.g. M1.3=53% down for both SLE and FLU because the composition of each vector can still be used to differentiate them. For example, even though the proportion and polarity of differentially expressed transcripts is identical between the two diseases for M1.3, the gene composition can still be disease-specific. The combination of gene-level and module-level analysis considerably increases resolution. Furthermore, it is possible to use 2, 3, 4, 5, 10, 15, 20, 25, 28 or more modules to differentiate diseases.

The term “gene” refers to a nucleic acid (e.g., DNA) sequence that includes coding sequences necessary for the production of a polypeptide (e.g.), precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “nucleic acid” refers to any nucleic acid containing molecule, including but not limited to, DNA, cDNA and RNA. In particular, the terms “a gene in Table X” refers to at least a portion or the full-length sequence listed in a particular table, as found hereinbelow. The gene may even be found or detected a genomic form, that is, it includes one or more intron(s). Genomic forms of a gene may also include sequences located on both the 5′ and 3′ end of the coding sequences that are present on the RNA transcript. These sequences are referred to as “flanking” sequences or regions. The 5′ flanking region may contain regulatory sequences such as promoters and enhancers that control or influence the transcription of the gene. The 3′ flanking region may contain sequences that influence the transcription termination, post-transcriptional cleavage, mRNA stability and polyadenylation.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

As used herein, the term “polymorphism” refers to the regular and simultaneous occurrence in a single interbreeding population of two or more alleles of a gene, where the frequency of the rarer alleles is greater than can be explained by recurrent mutation alone (typically greater than 1%).

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide protein) chain. The DNA sequence thus codes for the amino acid sequence.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tₘ of the formed hybrid, and the G:C ratio within the nucleic acids. A single molecule that contains pairing of complementary nucleic acids within its structure is said to be “self-hybridized.”

As used herein the term “stringency” is used in reference to the conditions of temperature, ionic strength, and the presence of other compounds such as organic solvents, under which nucleic acid hybridizations are conducted. Under “low stringency conditions” a nucleic acid sequence of interest will hybridize to its exact complement, sequences with single base mismatches, closely related sequences (e.g., sequences with 90% or greater homology), and sequences having only partial homology (e.g., sequences with 50-90% homology). Under “medium stringency conditions,” a nucleic acid sequence of interest will hybridize only to its exact complement, sequences with single base mismatches, and closely related sequences (e.g., 90% or greater homology). Under “high stringency conditions,” a nucleic acid sequence of interest will hybridize only to its exact complement, and (depending on conditions such a temperature) sequences with single base mismatches. In other words, under conditions of high stringency the temperature can be raised so as to exclude hybridization to sequences with single base mismatches.

As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences. Any probe used in the present invention may be labeled with any “reporter molecule,” so that it is detectable in any detection system, including, but not limited to enzyme (e.g., ELISA, as well as enzyme-based histochemical assays), fluorescent, radioactive, luminescent systems and the like. It is not intended that the present invention be limited to any particular detection system or label.

As used herein, the term “target,” refers to the region of nucleic acid bounded by the primers. Thus, the “target” is sought to be sorted out from other nucleic acid sequences. A “segment” is defined as a region of nucleic acid within the target sequence.

As used herein, the term “Southern blot” refers to the analysis of DNA on agarose or acrylamide gels to fractionate the DNA according to size followed by transfer of the DNA from the gel to a solid support, such as nitrocellulose or a nylon membrane. The immobilized DNA is then probed with a labeled probe to detect DNA species complementary to the probe used. The DNA may be cleaved with restriction enzymes prior to electrophoresis. Following electrophoresis, the DNA may be partially depurinated and denatured prior to or during transfer to the solid support. Southern blots are a standard tool of molecular biologists (Sambrook et al., Molecular Cloning: A Laboratory Manual, Cold Spring Harbor Press, NY, pp 9.31-9.58, 1989).

As used herein, the term “Northern blot” refers to the analysis of RNA by electrophoresis of RNA on agarose gels, to fractionate the RNA according to size followed by transfer of the RNA from the gel to a solid support, such as nitrocellulose or a nylon membrane. The immobilized RNA is then probed with a labeled probe to detect RNA species complementary to the probe used. Northern blots are a standard tool of molecular biologists (Sambrook, et al., supra, pp 7.39-7.52, 1989).

As used herein, the term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabeled antibodies.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”.

As used herein, the terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “real time PCR” as used herein, refers to various PCR applications in which amplification is measured during as opposed to after completion of the reaction. Reagents suitable for use in real time PCR embodiments of the present invention include but are not limited to TaqMan probes, molecular beacons, Scorpions primers or double-stranded DNA binding dyes.

As used herein, the terms “transcriptional upregulation,” “overexpression, and “overexpressed” refers to an increase in synthesis of RNA, by RNA polymerases using a DNA template. For example, when used in reference to the methods of the present invention, the term “transcriptional upregulation” refers to an increase of about 1 fold, 2 fold, 2 to 3 fold, 3 to 10 fold, and even greater than 10 fold, in the quantity of mRNA corresponding to a gene of interest detected in a sample derived from an individual predisposed to SLE as compared to that detected in a sample derived from an individual who is not predisposed to SLE. However, the system and evaluation is sufficiently specific to require less that a 2 fold change in expression to be detected. Furthermore, the change in expression may be at the cellular level (change in expression within a single cell or cell populations) or may even be evaluated at a tissue level, where there is a change in the number of cells that are expressing the gene. Changes of gene expression in the context of the analysis of a tissue can be due to either regulation of gene activity or relative change in cellular composition. Particularly useful differences are those that are statistically significant.

Conversely, the terms “transcriptional downregulation,” “underexpression” and “underexpressed” are used interchangeably and refer to a decrease in synthesis of RNA, by RNA polymerases using a DNA template. For example, when used in reference to the methods of the present invention, the term “transcriptional downregulation” refers to a decrease of least 1 fold, 2 fold, 2 to 3 fold, 3 to 10 fold, and even greater than 10 fold, in the quantity of mRNA corresponding to a gene of interest detected in a sample derived from an individual predisposed to SLE as compared to that detected in a sample derived from an individual who is not predisposed to such a condition or to a database of information for wild-type and/or normal control, e.g., fibromyalgia. Again, the system and evaluation is sufficiently specific to require less that a 2 fold change in expression to be detected. Particularly useful differences are those that are statistically significant.

Both transcriptional “upregulation”/overexpression and transcriptional “downregulation”/underexpression may also be indirectly monitored through measurement of the translation product or protein level corresponding to the gene of interest. The present invention is not limited to any given mechanism related to upregulation or downregulation of transcription.

The term “eukaryotic cell” as used herein refers to a cell or organism with membrane-bound, structurally discrete nucleus and other well-developed subcellular compartments. Eukaryotes include all organisms except viruses, bacteria, and bluegreen algae.

As used herein, the term “in vitro transcription” refers to a transcription reaction comprising a purified DNA template containing a promoter, ribonucleotide triphosphates, a buffer system that includes a reducing agent and cations, e.g., DTT and magnesium ions, and an appropriate RNA polymerase, which is performed outside of a living cell or organism.

As used herein, the term “amplification reagents” refers to those reagents (deoxyribonucleotide triphosphates, buffer, etc.), needed for amplification except for primers, nucleic acid template and the amplification enzyme. Typically, amplification reagents along with other reaction components are placed and contained in a reaction vessel (test tube, microwell, etc.).

As used herein, the term “diagnosis” refers to the determination of the nature of a case of disease. In some embodiments of the present invention, methods for making a diagnosis are provided which permit determination of SLE.

The present invention may be used alone or in combination with disease therapy to monitor disease progression and/or patient management. For example, a patient may be tested one or more times to determine the best course of treatment, determine if the treatment is having the intended medical effect, if the patient is not a candidate for that particular therapy and combinations thereof. The skilled artisan will recognize that one or more of the expression vectors may be indicative of one or more diseases and may be affected by other conditions, be they acute or chronic.

As used herein, the term “pharmacogenetic test” refers to an assay intended to study interindividual variations in DNA sequence related to, e.g., drug absorption and disposition (pharmacokinetics) or drug action (pharmacodynamics), which may include polymorphic variations in one or more genes that encode the functions of, e.g., transporters, metabolizing enzymes, receptors and other proteins.

As used herein, the term “pharmacogenomic test” refers to an assay used to study interindividual variations in whole-genome or candidate genes, e.g., single-nucleotide polymorphism (SNP) maps or haplotype markers, and the alteration of gene expression or inactivation that may be correlated with pharmacological function and therapeutic response.

As used herein, an “expression profile” refers to the measurement of the relative abundance of a plurality of cellular constituents. Such measurements may include, e.g., RNA or protein abundances or activity levels. The expression profile can be a measurement for example of the transcriptional state or the translational state. See U.S. Pat. Nos. 6,040,138, 5,800,992, 6,020,135, 6,033,860, relevant portions incorporated herein by reference. The gene expression monitoring system, include nucleic acid probe arrays, membrane blot (such as used in hybridization analysis such as Northern, Southern, dot, and the like), or microwells, sample tubes, gels, beads or fibers (or any solid support comprising bound nucleic acids). See, e.g., U.S. Pat. Nos. 5,770,722, 5,874,219, 5,744,305, 5,677,195 and 5,445,934, relevant portions incorporated herein by reference. The gene expression monitoring system may also comprise nucleic acid probes in solution.

The gene expression monitoring system according to the present invention may be used to facilitate a comparative analysis of expression in different cells or tissues, different subpopulations of the same cells or tissues, different physiological states of the same cells or tissue, different developmental stages of the same cells or tissue, or different cell populations of the same tissue.

As used herein, the term “differentially expressed: refers to the measurement of a cellular constituent varies in two or more samples. The cellular constituent can be either up-regulated in the test sample relative to the reference or down-regulated in the test sample relative to one or more references. Differential gene expression can also be used to distinguish between cell types or nucleic acids. See U.S. Pat. No. 5,800,992, relevant portions incorporated herein by reference.

Therapy or Therapeutic Regimen: In order to alleviate or alter a disease state, a therapy or therapeutic regimen is often undertaken. A therapy or therapeutic regimen, as used herein, refers to a course of treatment intended to reduce or eliminate the affects or symptoms of a disease. A therapeutic regimen will typically comprise, but is not limited to, a prescribed dosage of one or more drugs or surgery. Therapies, ideally, will be beneficial and reduce the disease state but in many instances the effect of a therapy will have non-desirable effects as well. The effect of therapy will also be impacted by the physiological state of the sample.

Modules display distinct “transcriptional behavior”. It is widely assumed that co-expressed genes are functionally linked. This concept of “guilt by association” is particularly compelling in cases where genes follow complex expression patterns across many samples. The present inventors discovered that transcriptional modules form coherent biological units and, therefore, predicted that the co-expression properties identified in our initial dataset would be conserved in an independent set of samples. Data were obtained for PBMCs isolated from the blood of twenty-one healthy volunteers. These samples were not used in the module selection process described above.
Keywords highly specific for M1.2 included Platelet, Aggregation or Thrombosis, and were associated with genes such as ITGA2B (Integrin alpha 2b, platelet glycoprotein IIb), PF4 (platelet factor 4), SELP (Selectin P) and GP6 (platelet glycoprotein 6).
Keywords highly specific for M1.3 included B-cell, Immunoglobulin or IgG and were associated with genes such as CD19, CD22, CD72A, BLNK (B cell linker protein), BLK (B lymphoid tyrosine kinase) and PAX5 (paired box gene 5, a B-cell lineage specific activator).
Keywords highly specific for M1.5 included Monocyte, Dendritic, CD14 or Toll-like and were associated with genes such as MYD88 (myeloid differentiation primary response gene 88), CD86, TLR2 (Toll-like receptor 2), LILRB2 (leukocyte immunoglobulin-like receptor B2) and CD163.
Keywords highly specific for M3.1 included Interferon, IFN-alpha, Antiviral, or ISRE and were associated with genes such as STAT1 (signal transducer and activator of transcription 1), CXCL10 (CXC chemokine ligand 10, IP-10), OAS2 (oligoadenylate synthetase 2) and MX2 (myxovirus resistance 2).

This contrasted pattern of term occurrence denotes the remarkable functional coherence of each module. Information extracted from the literature for all the modules that have been identified permit a comprehensive functional characterization of the PBMC system at a transcriptional level. A description of functional associations identified for each of the twenty-eight sample PBMC transcriptional modules is provided in Table 2.

TABLE 2
Complete Functional assessment of 28 transcriptional modules

| Module | Number of | | |
|---|---|---|---|
| I.D. | probe sets | Keyword selection | Assessment |
| M 1.1 | 69 | Ig, Immunoglobulin, | Plasma cells. Includes genes coding for |
| | | Bone, Marrow, PreB, | Immunoglobulin chains (e.g. IGHM, IGJ, |
| | | IgM, Mu. | IGLL1, IGKC, IGHD) and the plasma cell |
| | | | marker CD38. |
| M 1.2 | 96 | Platelet, Adhesion, | Platelets. Includes genes coding for platelet |
| | | Aggregation, | glycoproteins (ITGA2B, ITGB3, GP6, |
| | | Endothelial, Vascular | GP1A/B), and platelet-derived immune |
| | | | mediators such as PPPB (pro-platelet basic |
| | | | protein) and PF4 (platelet factor 4). |
| M 1.3 | 47 | Immunoreceptor, | B-cells. Includes genes coding for B-cell |
| | | BCR, B-cell, IgG | surface markers (CD72, CD79A/B, CD19, |
| | | | CD22) and other B-cell associated molecules: |
| | | | Early B-cell factor (EBF), B-cell linker |
| | | | (BLNK) and B lymphoid tyrosine kinase |
| | | | (BLK). |
| M 1.4 | 87 | Replication, | Undetermined. This set includes regulators |
| | | Repression, Repair, | and targets of cAMP signaling pathway (JUND, |
| | | CREB, Lymphoid, | ATF4, CREM, PDE4, NR4A2, VIL2), as well |
| | | TNF-alpha | as repressors of TNF-alpha mediated NF-KB |
| | | | activation (CYLD, ASK, TNFAIP3). |
| M 1.5 | 130 | Monocytes, | Myeloid lineage. Includes molecules expressed |
| | | Dendritic, MHC, | by cells of the myeloid lineage (CD86, CD163, |
| | | Costimulatory, | FCGR2A), some of which being involved in |
| | | TLR4, MYD88 | pathogen recognition (CD14, TLR2, MYD88). |
| | | | This set also includes TNF family members |
| | | | (TNFR2, BAFF). |
| M 1.6 | 28 | Zinc, Finger, P53, | Undetermined. This set includes genes coding |
| | | RAS | for signaling molecules, e.g. the zinc finger |
| | | | containing inhibitor of activated STAT (PIAS1 |
| | | | and PIAS2), or the nuclear factor of activated |
| | | | T-cells NFATC3. |
| M 1.7 | 127 | Ribosome, | MHC/Ribosomal proteins. Almost |
| | | Translational, 40S, | exclusively formed by genes coding MHC class |
| | | 60S, HLA | I molecules (HLA-A, B, C, G, E) + Beta 2- |
| | | | microglobulin (B2M) or Ribosomal proteins |
| | | | (RPLs, RPSs). |
| M 1.8 | 86 | Metabolism, | Undetermined. Includes genes encoding |
| | | Biosynthesis, | metabolic enzymes (GLS, NSF1, NAT1) and |
| | | Replication, Helicase | factors involved in DNA replication (PURA, |
| | | | TERF2, EIF2S1). |
| M 2.1 | 72 | NK, Killer, Cytolytic, | Cytotoxic cells. Includes cytotoxic T-cells amd |
| | | CD8, Cell-mediated, | NK-cells surface markers (CD8A, CD2, |
| | | T-cell, CTL, IFN-g | CD160, NKG7, KLRs), cytolytic molecules |
| | | | (granzyme, perforin, granulysin), chemokines |
| | | | (CCL5, XCL1) and CTL/NK-cell associated |
| | | | molecules (CTSW). |
| M 2.2 | 44 | Granulocytes, | Neutrophils. This set includes innate |
| | | Neutrophils, | molecules that are found in neutrophil granules |
| | | Defense, Myeloid, | (Lactotransferrin: LTF, defensin: DEAF1, |
| | | Marrow | Bacterial Permeability Increasing protein: BPI, |
| | | | Cathelicidin antimicrobial protein: CAMP . . . ). |
| M 2.3 | 94 | Erythrocytes, Red, | Erythrocytes. Includes hemoglobin genes |
| | | Anemia, Globin, | (HGBs) and other erythrocyte-associated genes |
| | | Hemoglobin | (erythrocytic alkirin: ANK1, Glycophorin C: |
| | | | GYPC, hydroxymethylbilane synthase: HMBS, |
| | | | erythroid associated factor: ERAF). |
| M 2.4 | 118 | Ribonucleoprotein, | Ribosomal proteins. Including genes encoding |
| | | 60S, nucleolus, | ribosomal proteins (RPLs, RPSs), Eukaryotic |
| | | Assembly, | Translation Elongation factor family members |
| | | Elongation | (EEFs) and Nucleolar proteins (NPM1, |
| | | | NOAL2, NAP1L1). |
| M 2.5 | 242 | Adenoma, Interstitial, | Undetermined. This module includes genes |
| | | Mesenchyme, | encoding immune-related (CD40, CD80, |
| | | Dendrite, Motor | CXCL12, IFNA5, IL4R) as well as |
| | | | cytoskeleton-related molecules (Myosin, |
| | | | Dedicator of Cytokenesis, Syndecan 2, Plexin |
| | | | C1, Distrobrevin). |
| M 2.6 | 110 | Granulocytes, | Myeloid lineage. Related to M 1.5. Includes |
| | | Monocytes, Myeloid, | genes expressed in myeloid lineage cells |
| | | ERK, Necrosis | (IGTB2/CD18, Lymphotoxin beta receptor, |
| | | | Myeloid related proteins 8/14 Formyl peptide |
| | | | receptor 1), such as Monocytes and |
| | | | Neutrophils. |
| M 2.7 | 43 | No keywords | Undetermined. This module is largely |
| | | extracted. | composed of transcripts with no known |
| | | | function. Only 20 genes associated with |
| | | | literature, including a member of the |
| | | | chemokine-like factor superfamily (CKLFSF8). |
| M 2.8 | 104 | Lymphoma, T-cell, | T-cells. Includes T-cell surface markers (CD5, |
| | | CD4, CD8, TCR, | CD6, CD7, CD26, CD28, CD96) and molecules |
| | | Thymus, Lymphoid, | expressed by lymphoid lineage cells |
| | | IL2 | (lymphotoxin beta, IL2-inducible T-cell kinase, |
| | | | TCF7, T-cell differentiation protein mal, |
| | | | GATA3, STAT5B). |
| M 2.9 | 122 | ERK, | Undetermined. Includes genes encoding |
| | | Transactivation, | molecules that associate to the cytoskeleton |
| | | Cytoskeletal, MAPK, | (Actin related protein 2/3, MAPK1, MAP3K1, |
| | | JNK | RAB5A). Also present are T-cell expressed |
| | | | genes (FAS, ITGA4/CD49D, ZNF1A1). |
| M 2.10 | 44 | Myeloid, | Undetermined. Includes genes encoding for |
| | | Macrophage, | Immune-related cell surface molecules (CD36, |
| | | Dendritic, | CD86, LILRB), cytokines (IL15) and |
| | | Inflammatory, | molecules involved in signaling pathways |
| | | Interleukin | (FYB, TICAM2-Toll-like receptor pathway). |
| M 2.11 | 77 | Replication, Repress, | Undetermined. Includes kinases (UHMK1, |
| | | RAS, | CSNK1G1, CDK6, WNK1, TAOK1, CALM2, |
| | | Autophosphorylation | PRKCI, ITPKB, SRPK2, STK17B, DYRK2, |
| | | Oncogenic | PIK3R1, STK4, CLK4, PKN2) and RAS family |
| | | | members (G3BP, RAB14, RASA2, RAP2A, |
| | | | KRAS). |
| M 3.1 | 80 | ISRE, Influenza, | Interferon-inducible. This set includes |
| | | Antiviral, IFN- | interferon-inducible genes: antiviral molecules |
| | | gamma, IFN-alpha, | (OAS1/2/3/L, GBP1, G1P2, EIF2AK2/PKR, |
| | | Interferon | MX1, PML), chemokines (CXCL10/IP-10), |
| | | | signaling molecules (STAT1, STAt2, IRF7, |
| | | | ISGF3G). |
| M 3.2 | 230 | TGF-beta, TNF, | Inflammation I. Includes genes encoding |
| | | Inflammatory, | molecules involved in inflammatory processes |
| | | Apoptotic, | (e.g. IL8, ICAM1, C5R1, CD44, PLAUR, |
| | | Lipopolysaccharide | IL1A, CXCL16), and regulators of apoptosis |
| | | | (MCL1, FOXO3A, RARA, BCL3/6/2A1, |
| | | | GADD45B). |
| M 3.3 | 230 | Granulocyte, | Inflammation II. Includes molecules inducing |
| | | Inflammatory, | or inducible by Granulocyte-Macrophage CSF |
| | | Defense, Oxidize, | (SPI1, IL18, ALOX5, ANPEP), as well as |
| | | Lysosomal | lysosomal enzymes (PPT1, CTSB/S, CES1, |
| | | | NEU1, ASAH1, LAMP2, CAST). |
| M 3.4 | 323 | No keyword | Undetermined. Includes protein phosphates |
| | | extracted | (PPP1R12A, PTPRC, PPP1CB, PPM1B) and |
| | | | phosphoinositide 3-kinase (PI3K) family |
| | | | members (PIK3CA, PIK32A, PIP5K3). |
| M 3.5 | 19 | No keyword | Undetermined. Composed of only a small |
| | | extracted | number of transcripts. Includes hemoglobin |
| | | | genes (HBA1, HBA2, HBB). |
| M 3.6 | 233 | Complement, Host, | Undetermined. This very large set includes T- |
| | | Oxidative, | cell surface markers (CD101, CD102, CD103) |
| | | Cytoskeletal, T-cell | as well as molecules ubiquitously expressed |
| | | | among blood leukocytes (CXRCR1: fraktalkine |
| | | | receptor, CD47, P-selectin ligand). |
| M 3.7 | 80 | Spliceosome, | Undetermined. Includes genes encoding |
| | | Methylation, | proteasome subunits (PSMA2/5, PSMB5/8); |
| | | Ubiquitin, Beta- | ubiquitin protein ligases HIP2, STUB1, as well |
| | | catenin | as components of ubiqutin ligase complexes |
| | | | (SUGT1). |
| M 3.8 | 182 | CDC, TCR, CREB, | Undetermined. Includes genes encoding for |
| | | Glycosylase | several enzymes: aminomethyltransferase, |
| | | | arginyltransferase, asparagines synthetase, |
| | | | diacylglycerol kinase, inositol phosphatases, |
| | | | methyltransferases, helicases . . . |
| M 3.9 | 261 | Chromatin, | Undetermined. Includes genes encoding for |
| | | Checkpoint, | protein kinases (PRKPIR, PRKDC, PRKCI) |
| | | Replication, | and phosphatases (e.g. PTPLB, PPP1R8/2CB). |
| | | Transactivation | Also includes RAS oncogene family members |
| | | | and the NK cell receptor 2B4 (CD244). |

The present includes the implementation of a module-level microarray data analysis strategy and the characterization of immune transcriptional vectors. The modular decomposition of blood leukocyte transcriptional profiles improves the understanding of disease pathogenesis, leading for instance to the identification of a signature of immunosuppression common to patients with metastatic melanoma and liver transplant recipients. It is demonstrated herein that immune transcriptional vectors can be used as diagnostic markers and indicators of disease severity.

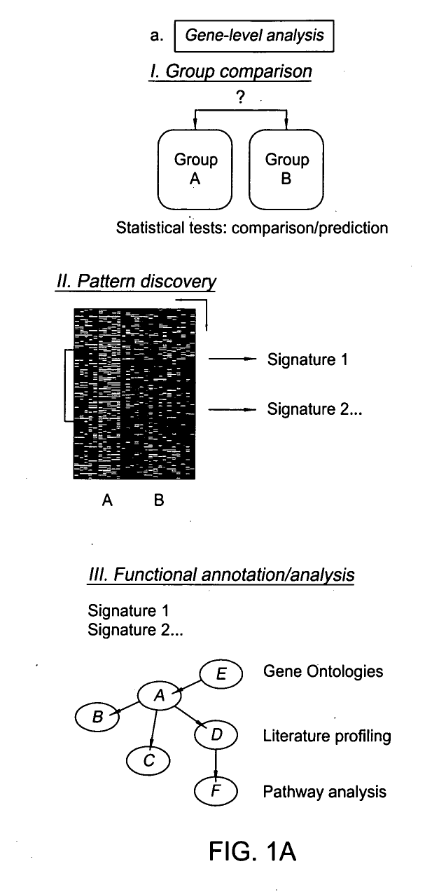

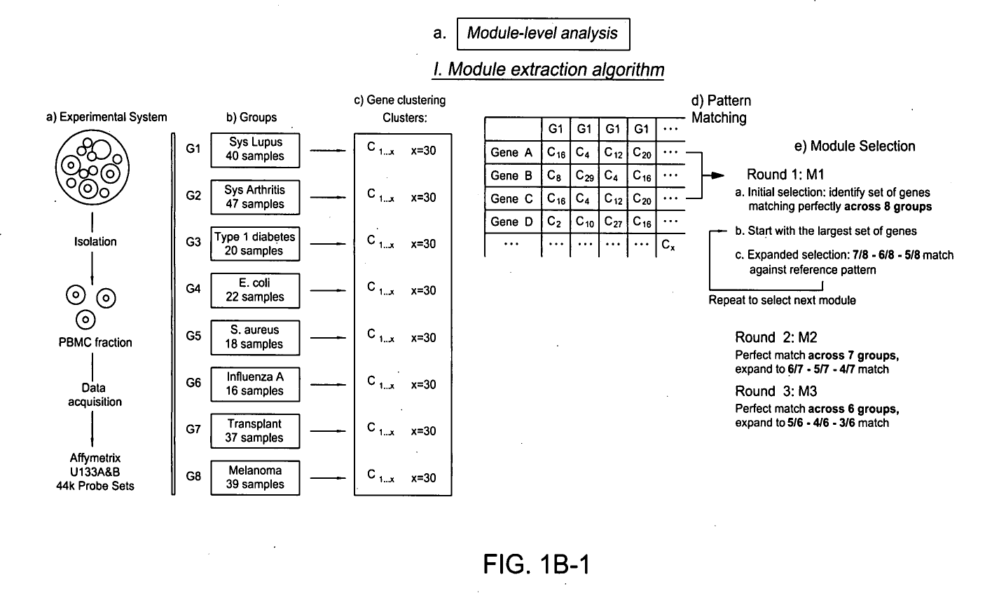

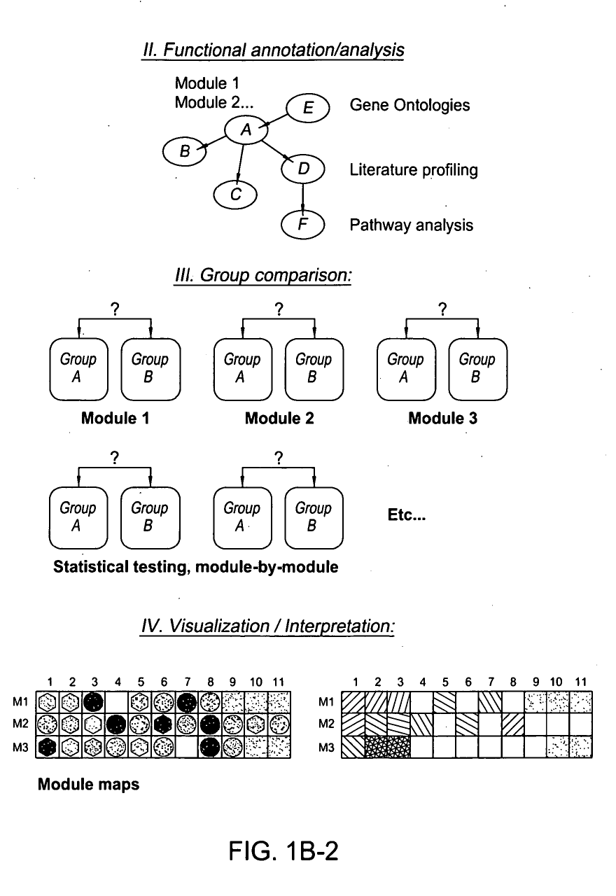

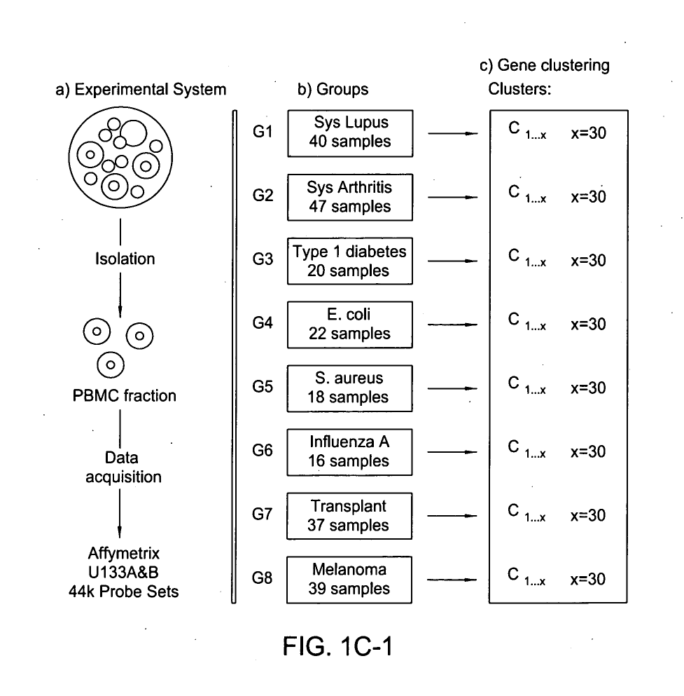

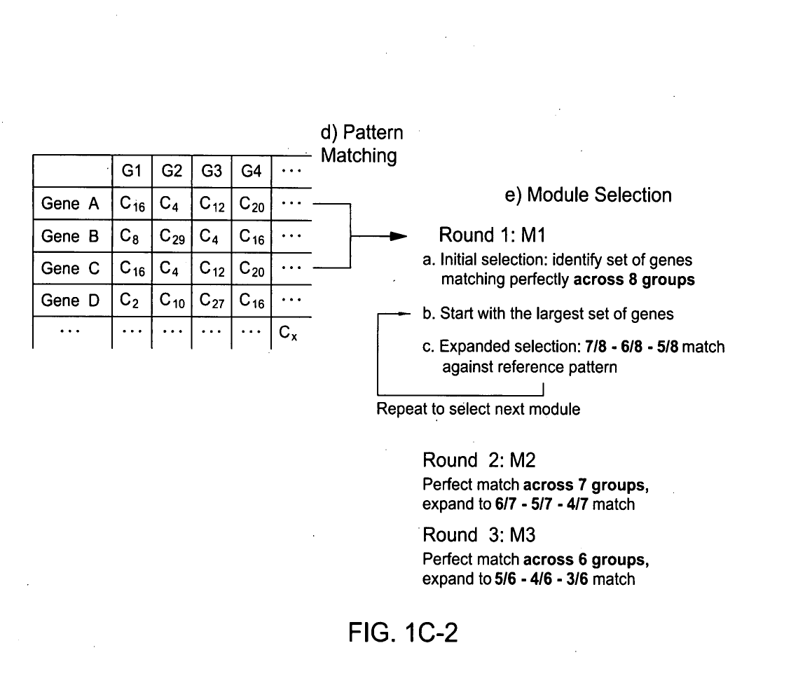

Prior Art microarray data mining strategy. Results from “traditional” microarray analyses are notoriously noisy and difficult to interpret. Conventional gene-level microarray analyses includes three basic steps (FIG. 1a): I. Group comparison: Differentially expressed genes are identified by comparing the different study groups. II. Pattern discovery: Differentially expressed genes are grouped according to their transcriptional profile across multiple conditions. III. Functional annotation/analysis: Functional relationships between genes forming transcriptional signatures are uncovered using ontology-based and/or literature-based analysis tools. This gene-level analysis approach is supported by popular microarray data mining software and is commonly used in microarray publications (e.g., (Borovecki et al., 2005; Calvano et al., 2005; Ockenhouse et al., 2005; Willinger et al., 2005)).

In contrast, the microarray data mining strategy described herein relies instead on the initial characterization of transcriptional modules that serve as a basis to carry out independent statistical group comparisons at a later stage of the analysis (FIG. 1b): I. Module extraction: Sets of coordinately expressed genes are identified using a custom module extraction algorithm (FIG. 1c for details and methods taught hereinbelow). Importantly, the analysis does not take into consideration differences in gene expression levels between study groups; it focuses instead on complex gene expression patterns that arise from biological variations (e.g., inter-individual variations among a patient population, or variations introduced by different treatments). II. Functional annotation/analysis: Functional relationships between genes forming transcriptional modules are uncovered using ontology-based and/or literature-based analysis tools. III. Group comparison: Differentially expressed genes are identified at this stage by comparing study groups on a module-by-module basis. Notably, carrying out statistical comparisons at the level of each module avoids the noise generated when thousands of tests are performed across an entire set of microarray probes. IV. Visualization/Interpretation: Finally, data are interpreted by mapping global transcriptional changes occurring across all modules.

The microarray analysis described herein is based on the identification of sets of coordinately expressed transcripts, or transcriptional modules, which are derived using a data mining algorithm; i.e. this “data-driven” selection process does not require any intervention from the part of the investigator and does not involve any a priori knowledge of gene function. Transcriptional modules are subjected to functional analysis only after the selection process has taken place. Notably, sets of modules are specific for the biological system from which they have been derived. As a result, modules constitute a framework for analyzing data obtained in the context of a defined biological system (i.e. blood transcriptional modules will not permit to analyze data obtained from another tissue; a different set of modules would have to be generated).

Identification of transcriptional modules in peripheral blood cells: The modular mining strategy described above was implemented on a peripheral blood mononuclear cell (PBMC) transcriptional dataset. Identification of blood leukocyte transcriptional modules was based on the analysis of an extensive collection of microarray gene expression profiles generated for a wide range of diseases: systemic juvenile idiopathic arthritis, systemic lupus erythematosus (SLE), type I diabetes, metastatic melanoma, acute infections (Escherichia coli, Staphylococcus aureus, Influenza A), and liver transplant recipients undergoing immunosuppressive therapy. A total of 239 PBMC transcriptional profiles were acquired using Affymetrix U133A and U133B GeneChips (>44,000 probesets). Transcriptional modules were extracted using a custom algorithm (see Methods section for details). For this analysis 4742 transcripts were selected that were distributed among 28 modules (a complete list is provided in Supplementary Table 1). Each module was assigned a unique identifier indicating the round and order of selection (i.e. M3.1 was the first module identified in the third round of selection).

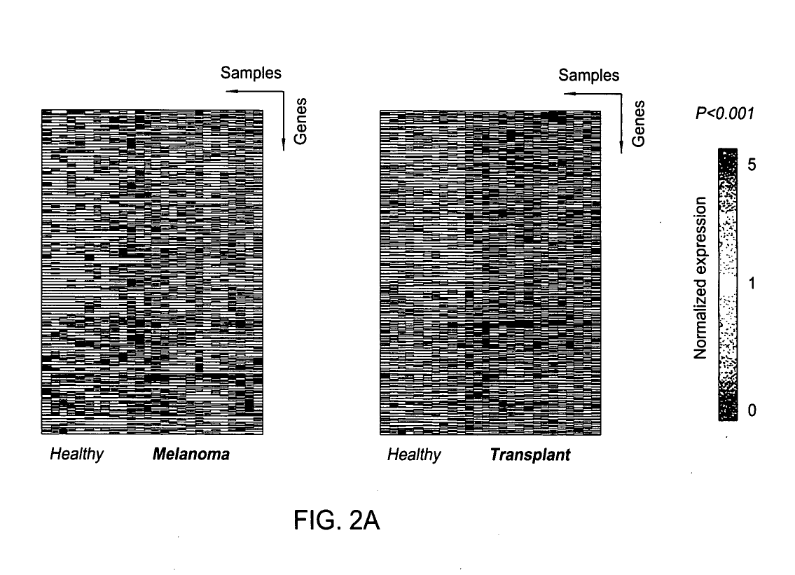

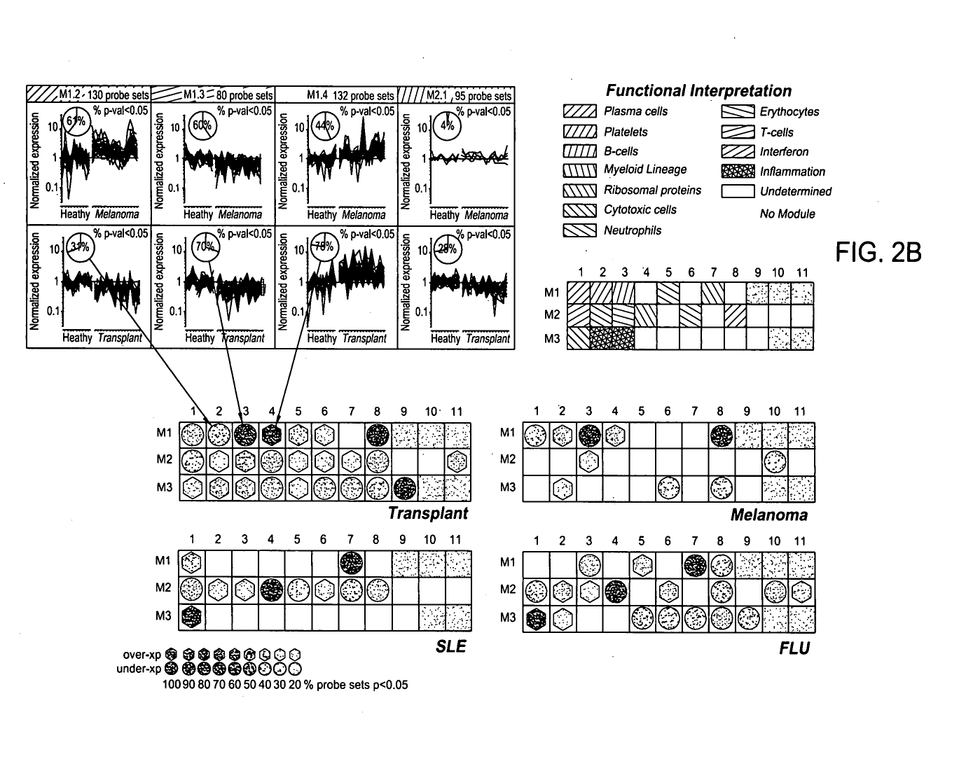

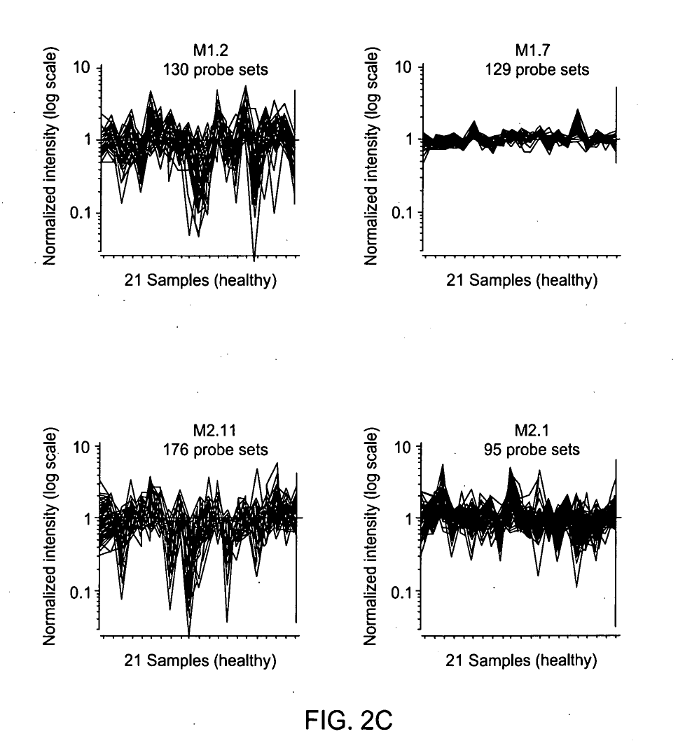

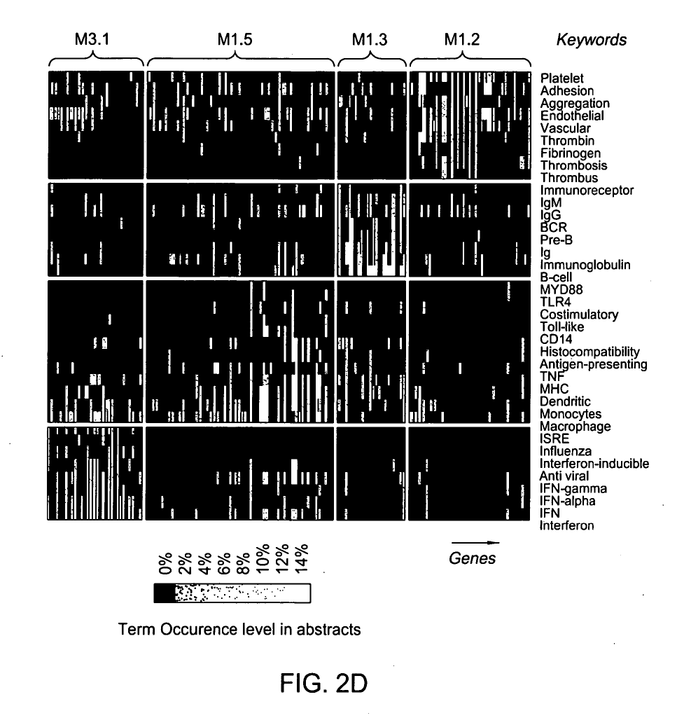

Functional characterization of PBMC transcriptional modules. Modules form coherent transcriptional units and, therefore, it was found that the co-expression properties identified in the initial dataset would be conserved in an independent set of samples. This observation was confirmed in a set of data were obtained for PBMCs isolated from the blood of 21 subjects that were not used in the module selection process described above (FIG. 2c). Next, each module was characterized functionally (FIG. 1b: Step 11). Keyword occurrence in PubMed abstracts associated with the genes forming each module was analyzed by literature profiling (described in (Chaussabel and Sher, 2002). Differential keyword distribution is illustrated in four modules in FIG. 2d), and a description of functional associations identified for each of the 28 PBMC transcriptional modules is provided in Supplementary Table 2. This analysis demonstrates that transcriptional modules form coherent functional units. In 14 out of the 28 PBMC modules the present invention was used to associate some of the genes with pathways and cell types involved in immune processes. Functional convergence was also observed in the remaining 14 modules, but actual implications remain unclear (e.g., M2.5 includes genes encoding immune-related—CD40, CD80, CXCL12, IFNA5, IL4R—as well as cytoskeleton-related molecules—Myosin, Dedicator of Cytokenesis, Syndecan 2, Plexin C1, Distrobrevin; or M2.11, which includes a number of kinases—UHMK1, CSNK1G1, CDK6, WNK1, TAOK1, CALM2, PRKCI, ITPKB, SRPK2, STK17B, DYRK2, PIK3R1, STK4, CLK4, PKN2—and RAS family members—G3BP, RAB14, RASA2, RAP2A, KRAS).

Module-level analysis of PBMC transcriptional profiles in health and disease. Gene-level analysis: PBMC microarray transcriptional profiles were obtained from 16 patients with metastatic melanoma and 16 liver transplant recipients receiving immunosuppressive drug treatments and matched healthy control subjects. The gene-level analysis described in FIG. 1a identified differentially expressed transcripts between patients and respective healthy control group (Mann Whitney U test, p<0.001). Hierarchical clustering defined two signatures in each group, separating over-expressed and under-expressed transcripts (FIG. 2a).

Module-level analysis: This analysis was carried out using PBMC transcriptional modules which were extracted and characterized in advance (Steps I and II of FIG. 1b). Statistical group comparisons between patient and healthy groups were performed independently, on a module-by-module basis (FIG. 1b: Step III, Mann Whitney U test, p<0.05). For each module, transcriptional profiles of differentially expressed genes were represented on a graph, with a pie-chart indicating the proportion of differentially expressed transcripts (FIG. 2b, e.g., 61% of the 130 transcripts forming module M1.2 are over-expressed in patients with melanoma compared to healthy controls). Interestingly, differentially expressed genes in each module were predominantly either under-expressed or over-expressed (FIG. 2b, Supplementary Table 2). Since modules were not extracted based on differences in expression levels between groups, the fact that changes in gene expression are almost unanimous reflects the consistency of transcriptional behavior characterizing each module.

Mapping modular transcriptional changes: Data visualization is paramount for the interpretation of complex datasets and these were used to illustrate, graphically, global modular changes (FIG. 1b: Step 1V). Module-level data were represented by spots aligned on a grid, with each position corresponding to a different module (FIG. 2c). The spot intensity indicates the proportion of genes significantly changed for each module. The spot color indicates the polarity of the change (red: proportion of over-expressed genes, blue: proportion of under-expressed genes). This representation permits a global assessment of perturbations of the PBMC transcriptional system. A modules' coordinates can be associated to functional annotations to facilitate data interpretation (FIG. 2d, Supplementary Table 2).

Modular analysis reveal disease-specific perturbations of PBMC transcriptional profiles: Module maps were generated for four groups of patients compared to their respective control groups composed of healthy donors who were matched for age and sex (22 patients with SLE, 16 with acute influenza infection, 16 with metastatic melanoma and 16 liver transplant recipients were compared to control groups composed of 10 to 12 healthy subjects). Each module has one of four possible states depending whether its genes are: over-expressed (red spot), under-expressed (blue spot), both over- and under-expressed (purple spot—not observed here), not changed (empty). Remarkably, results for M1.1 and M1.2 alone sufficed to distinguish all four diseases (M1.1/M1.2: SLE=+/0; FLU=0/0; Melanoma=−/+; transplant=−/−). A number of genes in M3.2 (“inflammation”) were over-expressed in all diseases (particularly so in the transplant group), while genes in M3.1 (interferon) were over-expressed in patients with SLE, influenza infection and, to some extent, transplant recipients. M2.1 and M2.8 includes, respectively, cytotoxic cells and T-cell transcripts that are under-expressed in lymphopenic SLE patients and transplant recipients treated with immunosuppressive drugs. Thus, the invention was used to demonstrate that diseases are characterized by unique combinations of modular transcriptional changes. Furthermore, it was found that in comparison to the heatmaps obtained by carrying out conventional gene-level analysis (FIG. 2a), applying the proposed module-level mining strategy on the same set of data yielded an elaborate and interpretable representation of microarray results (FIG. 2c).

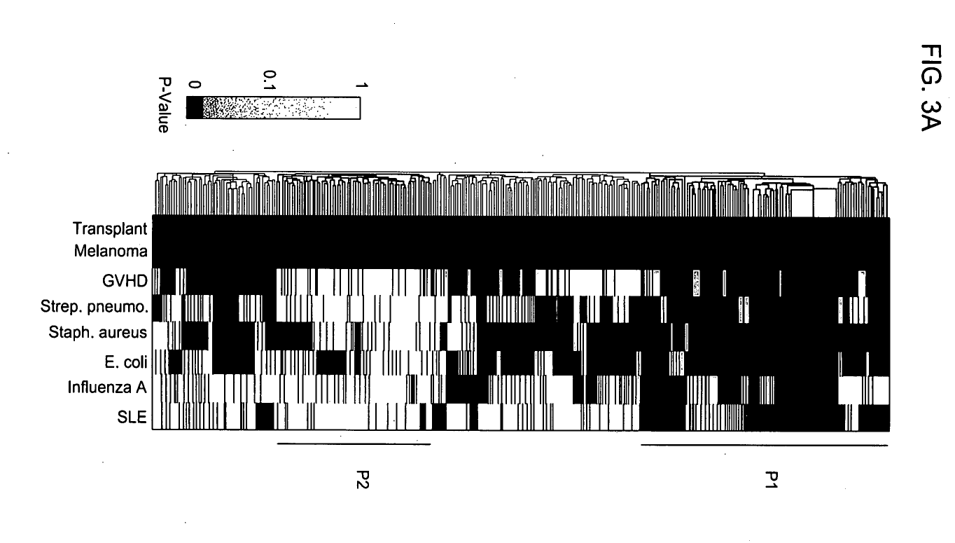

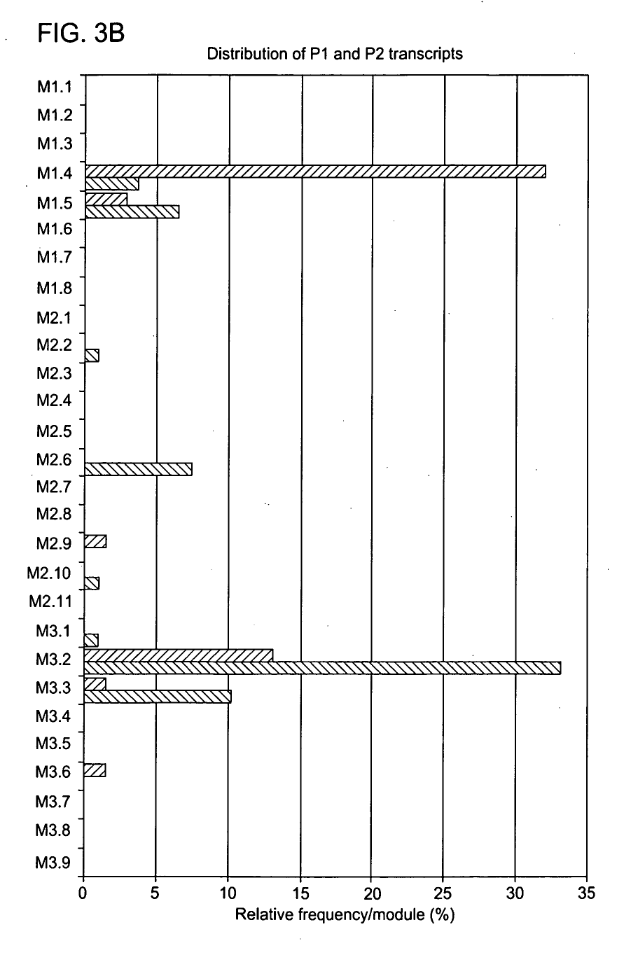

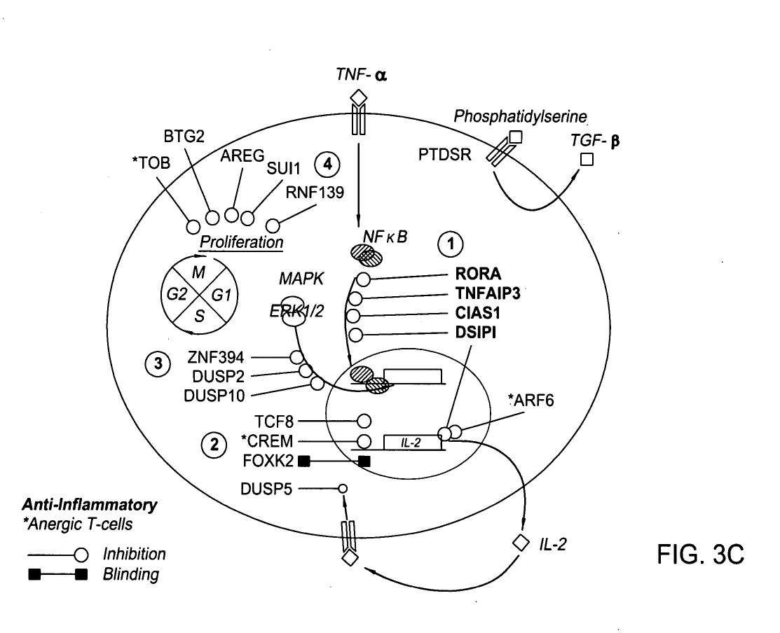

Gaining insights into disease pathogenesis: Sets of transcripts are preferentially over-expressed in patients with metastatic melanoma and liver transplant recipients under treatment with immunosuppressive drugs: Decomposing microarray data in sets of pre-defined transcriptional modules can provide novel insights into mechanisms of disease pathogenesis. It was found that an important proportion of transcripts forming M1.4 were changed both in patients with melanoma and in liver transplant recipients. No changes were on the other hand detected in patients with acute influenza infection and lupus (FIG. 2c). These findings prompted a more in depth investigation. Blood microarray data were generated from a total of 35 patients with metastatic melanoma, 39 liver transplant recipients and 25 healthy subjects. The extent to which similarities observed between patients with metastatic melanoma and liver transplant recipients were specific to these two groups of patients was determined. Statistical group comparison was carried out at the gene-level between patients and healthy controls. This analysis identified 323 transcripts that were significantly overexpressed in both liver transplant recipients and patients with metastatic melanoma (Mann-Whitney U test, p <0.01, filtered >1.25 fold change). Next, group comparisons for these transcripts were carried out using samples from patients with systemic lupus erythematosus (SLE), acute infections (Streptococcus pneumoniae, Staphylococcus aureus, Escherichia. coli, and influenza A) or graft versus host disease (GVHD) vs. respective healthy control groups. The p-values generated by this analysis were grouped by hierarchical clustering based on similarities in patterns of significance (FIG. 3a; this approach is described in details in (Chaussabel et al., 2005). Sets of genes that were ubiquitously overexpressed formed the pattern P1 (Supplementary Table 3); conversely transcripts more specifically expressed in patients with melanoma and transplant recipients formed the pattern P2 (Supplementary Table 4).

Thus, it was found that genes forming transcriptional signatures common to the melanoma and transplant groups can be partitioned into distinct sets based on two properties: (1) coordinated expression (transcriptional modules: FIG. 2b); and (2) change in expression across diseases (significance patterns: FIG. 3a). To cross-validate the results from these two different approaches the modular distribution of ubiquitous (P1) and specific (P2) PBMC transcriptional signatures was determined. FIG. 3b shows that the distribution of P1 and P2 across the 28 PBMC transcriptional modules that have been identified to date is not random. Indeed, P1 transcripts are preferentially found among M3.2 (characterized by transcripts related to inflammation), whereas M1.4 transcripts almost exclusively belong to P2, which includes genes that are more specifically overexpressed in patients with melanoma and liver transplant recipients.

Patients with melanoma display a transcriptional signature of immunosuppression common to liver transplant recipients: Focus was placed on genes that were most specifically overexpressed in melanoma and transplant groups (P2). From the 69 probe sets, 55 unique gene identifiers were found. A query against a literature database indexed by gene, was developed to aid in the interpretation of microarray gene expression data, identified 6527 publications associated with 47 genes, 30 of which were associated with more than ten publications. It was found that a remarkable functional convergence among the genes forming this signature (FIG. 3c). The module includes genes encoding molecules that display immunoregulatory activity: (1) inhibitors of NF-kB pathway such as TNFAIP3 or CIAS1 (Cryopyrin), which regulate NF-kappa B activation and production of proinflammatory cytokines. Mutations of this gene have been identified in several inflammatory disorders (Agostini et al., 2004). DSIPI, a leucine zipper protein, is known to mediate the immunosuppressive effects of glucocorticoids and IL-10 by interfering with a broad range of signaling pathways (NF-kappa B, NFAT/AP-1, MEK, ERK 1/2), leading to the general inhibition of inflammatory responses in macrophages and down-regulation of the IL-2 receptor in T cells. Notably, the expression of DSIPI in immune cells was found to be augmented after drug treatment (dexamethasone) (D'Adamio et al., 1997) or long term exposure to tumor cells (Burkitt Lymphoma) (Berrebi et al., 2003). (2) Inhibitors of MAP kinase pathway: for instance, dual specificity phosphatases 2, 5 and 10 (DUSP2, DUSP5 and DUSP10) interfere with the MAP kinases ERK1/2, which are known targets of calcineurin inhibitors (such as Tacrolimus/FK506). (3) Inhibitors of IL2 production: CREM, FOXK2 and TCF8 directly bind the IL-2 promoter and can contribute to the repression of IL-2 production in anergic T cells (Powell et al., 1999). Interestingly, DUSP5 was found to have a negative feedback role in IL-2 signaling in T-cells (Kovanen et al., 2003). (4) Inhibitors of cell proliferation (e.g., BTG2, TOB1, AREG, SUI1 and RNF139). Other molecules, such as BHLHB2 (Stra13) negatively regulate lymphocyte development and function in vivo (Seimiya et al., 2004).

Thus, patients with metastatic melanoma display a signature of immunosuppression similar to the signature induced by pharmacological regimen in liver transplant recipients.

Biomarker discovery I: characterization of microarray immune transcriptional vectors in the blood of patients with systemic lupus. Blood serves as a reservoir for cells responding to signals acquired in the bloodstream and in the tissues from which they migrate. It constitutes therefore an accessible source of clinically-relevant information. Indeed, microarray gene expression data generated from blood not only provide valuable insights into mechanisms of disease pathogenesis but constitute also a promising source of biomarkers. The difficulty, however, lies in the extraction of indicators of potential clinical value from the vast amounts of data generated by genome-wide expression scans. Modular transcriptional data was used as the foundation of a biomarker discovery strategy and used to illustrate the implementation of this novel approach using a dataset generated from a cohort of pediatric patients with systemic lupus erythematosus (SLE).

Blood transcriptional signatures of Lupus: SLE is an autoimmune disease characterized by dysregulation of innate and adaptive immunity (Carroll, 2004; Grammer and Lipsky, 2003; Kong et al., 2003; Manderson et al., 2004; Manzi et al., 2004; Nambiar et al., 2004). Gene-level analyses have been carried out on peripheral blood mononuclear cells obtained from pediatric and adult SLE patients (Baechler et al., 2003; Bennett et al., 2003; Crow et al., 2003; Kirou et al., 2004). Using an earlier generation of Affymetrix arrays (˜12,600 probe sets), a type I interferon (IFN) signature was identified in all active pediatric patients (Bennett et al., 2003). This data confirmed that activation of the type I IFN pathway is a universal feature of pediatric SLE. This analysis also revealed the presence of neutrophil, immunoglobulin (Ig) and lymphocyte signatures that correlated with the presence of low density granulocytes, plasma cell precursors and a reduction in lymphocyte numbers in SLE blood, respectively (Bennett et al., 2003). In the present study, these signatures were reflected at the module-level by significant changes observed in modules M3.1, M2.2, M1.1 and M2.8 (interferon-inducible, neutrophils, plasma cells and T-lymphocytes, respectively). These results were obtained in a new cohort of pediatric lupus patients sampled at the time of diagnosis and before initiation of treatment, analyzing over 44,000 transcripts on Affymetrix U133 genechips. It was found that in addition transcriptional changes in 7 other modules (FIG. 2b: M1.7, M2.1, M2.3, M2.4, M2.5, M2.6, and M2.7). Interestingly, M1.7 and M2.4 include a number of transcripts encoding ribosomal protein family members which expression was recently found altered in the context of acute infection and sepsis (Calvano et al., 2005; Thach et al., 2005)—see also FIG. 2b: acute influenza infection).

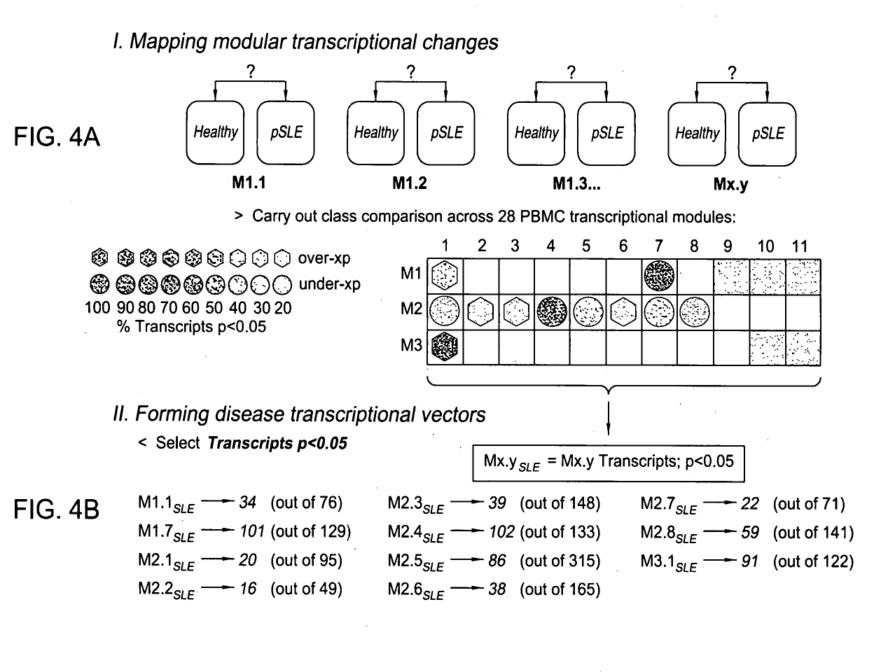

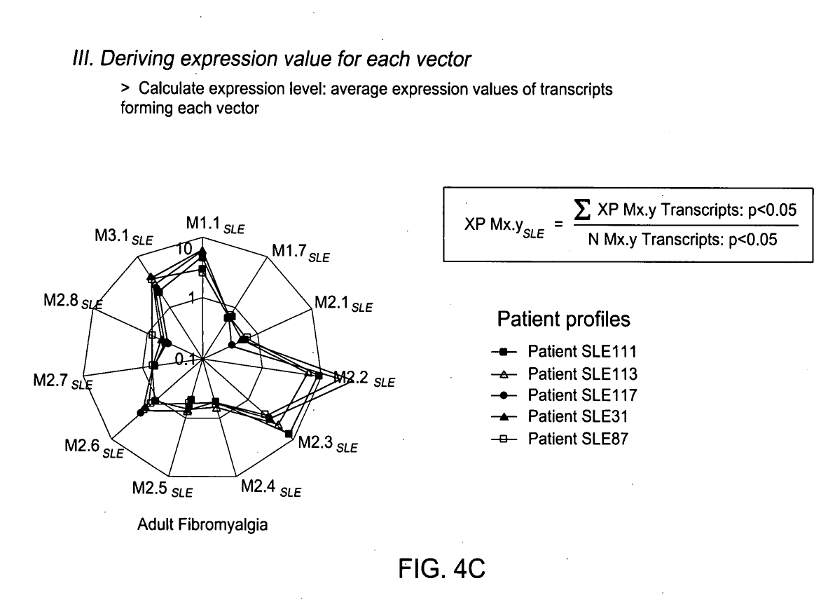

Assembly of transcriptional vectors: The biomarker discovery strategy developed relies on the initial selection of modules that are changed significantly in comparison to control subjects (e.g., healthy volunteers). In this example, 11 modules were used for which changes were observed in untreated pediatric SLE patients (FIG. 4, step I). “Transcriptional vectors” were then formed through the selection of genes that were significantly changed compared to healthy subjects for each of the 11 modules (FIG. 4, step II). Expression levels were subsequently derived by averaging the values obtained for the subset of transcripts forming each vector (FIG. 4, step III). Patient profiles can then be represented by plotting expression levels obtained for each of these vectors on a graph (e.g., on a radar plot). A set of vectors is disease-specific by construction, since it results from two round of selection, first at the module level (Step I: e.g., 11 out of 28 modules in SLE), and then at the gene level (Step 11: p<0.05 in disease vs. healthy control groups).

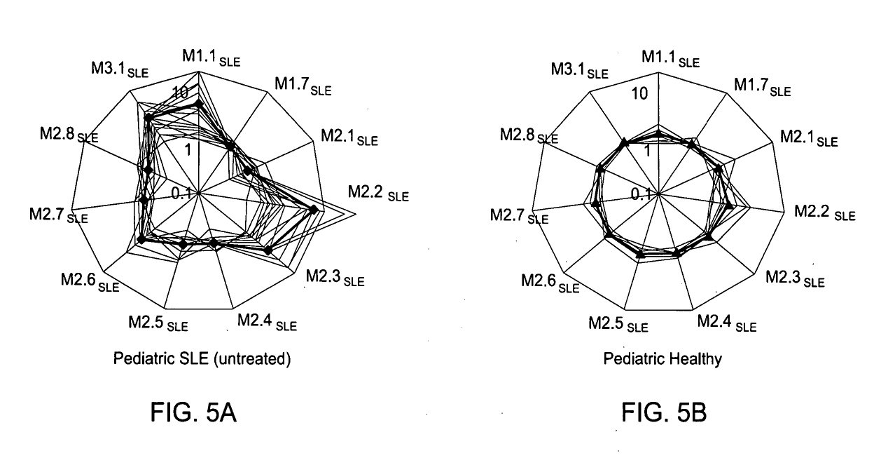

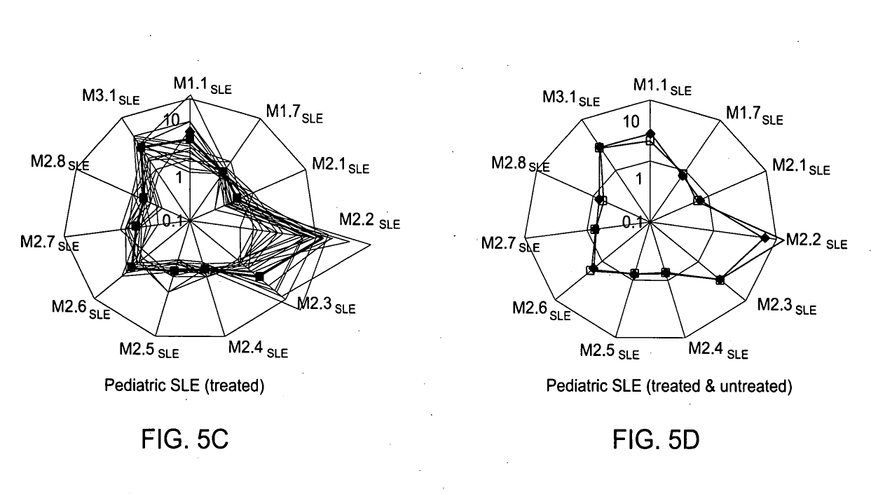

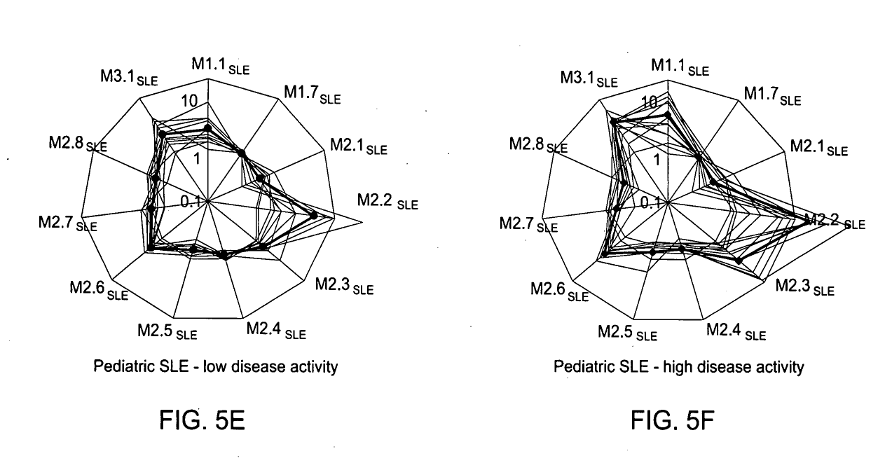

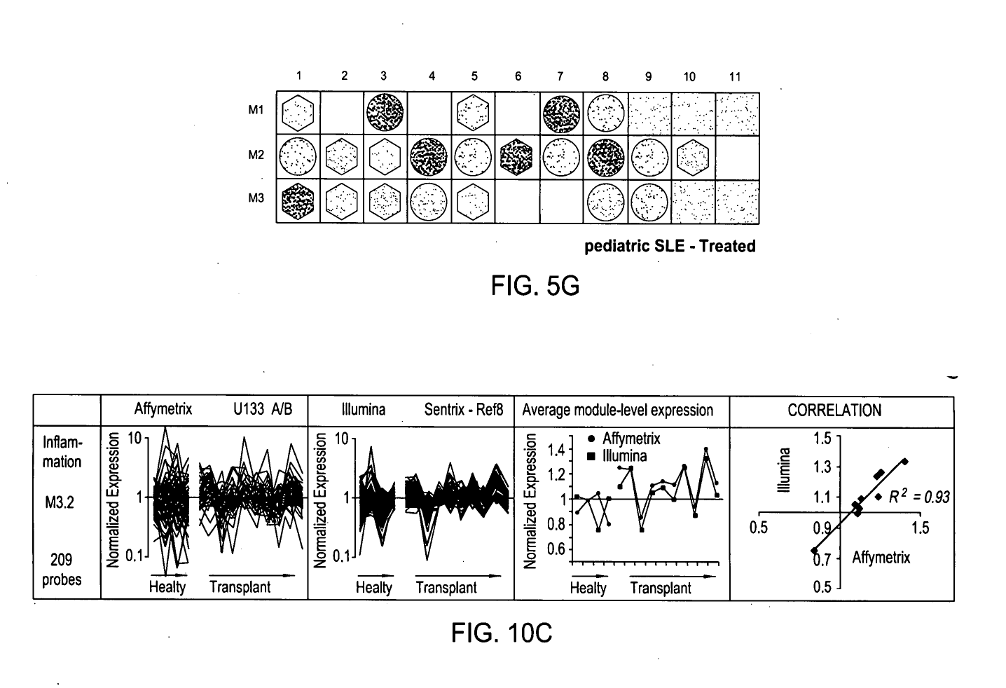

Lupus blood transcriptional vectors: Profiles were derived using the set of SLE vectors obtained above for the entire cohort of untreated pediatric SLE patients (FIG. 5a: each line is one patient, the thicker line is an average for all patients), while FIG. 5b displays on the same vectors the regular pattern characteristic of healthy volunteers. This master set of markers can be used as a reference to derive expression levels for other sets of samples. Patient profiles were generated for an independent set of children with SLE treated orally with steroids (patients receiving high dose steroids were excluded) and/or cytotoxic drugs and/or hydroxychloroquine (N=31; FIG. 5c). Interestingly, average profiles for both treated and untreated patient cohorts were almost superimposable (FIG. 5d). This unexpected result can be explained by the fact that both groups of patients presented similar disease activity as measured by the clinical index SLEDAI (SLE disease activity index-untreated patients average=11.5±7.9; treated patients=9.4±6.4, Student's t-Test p=0.3). Indeed, stratification of the samples based on disease activity and regardless of treatment yielded contrasting profiles: samples from patients with mild disease presented a more regular profile compared to either treated or untreated patient cohorts (FIG. 5e, SLEDAI [0-6]); while patients with high disease activity presented an exacerbated profile (FIG. 5f, SLEDAI [14-28]). Thus, these results demonstrate that immune transcriptional vectors identified in SLE patients are linked directly to the disease process. Notably an effect of treatment could be observed when mapping modular transcriptional changes for treated pediatric SLE patients (FIG. 5g). However, the core disease signature obtained in untreated patients remains.

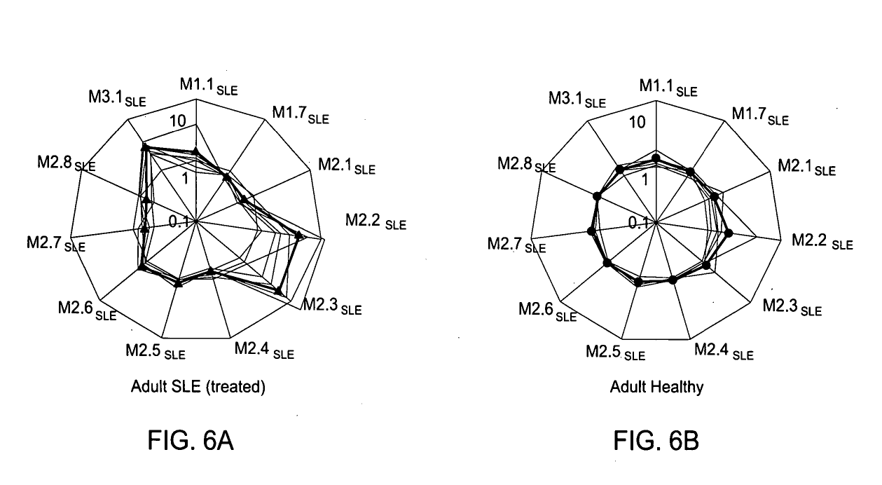

Relevance of transcriptional vectors as diagnostic markers. Using untreated pediatric SLE vectors as a reference, gene profiles were generated for adult patients with SLE. These subjects presented perturbed expression patterns consistent with those observed in pediatric patients (FIG. 6a). This is in contrast with adult patients with fibromyalgia who present few of the characteristics of an SLE signature (FIG. 6b), and resemble more healthy adults (FIG. 6c). This finding is notable since patients with fibromyalgia present symptoms which are consistent with systemic Lupus, leading in some cases to a diagnosis dilemma (Blumenthal, 2002). These results illustrate the potential diagnostic value of immune transcriptional vectors derived from the microarray analysis of patient blood.

Biomarker discovery II: multivariate microarray scores for the assessment of disease severity in patients with systemic lupus. SLE is a disease characterized by flares of high morbidity. At least 6 composite measures of SLE global disease activity are available (Bae et al., 2001; Bencivelli et al., 1992; Bombardier et al., 1992; Hay et al., 1993; Liang et al., 1989; Petri et al., 1999). These instruments provide metrics to document and quantify disease activity and have been used in clinical trials. Some of the included measures, however, are not easy to obtain. Conversely, given the heterogeneous nature of the clinical disease, not all SLE manifestations are computed in these instruments, making the overall assessment of the patient condition difficult. One purpose was to establish an objective disease activity index based on blood leukocyte microarray transcriptional data.

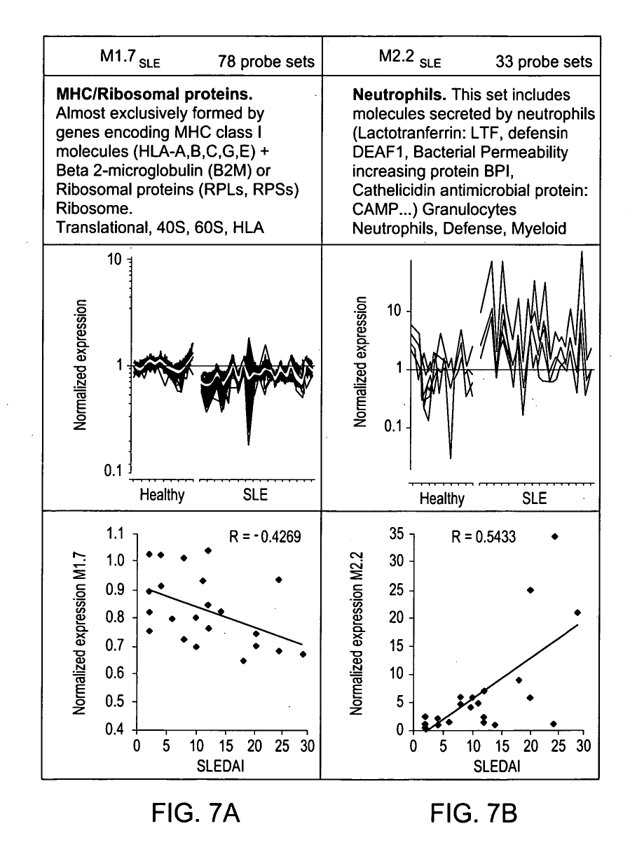

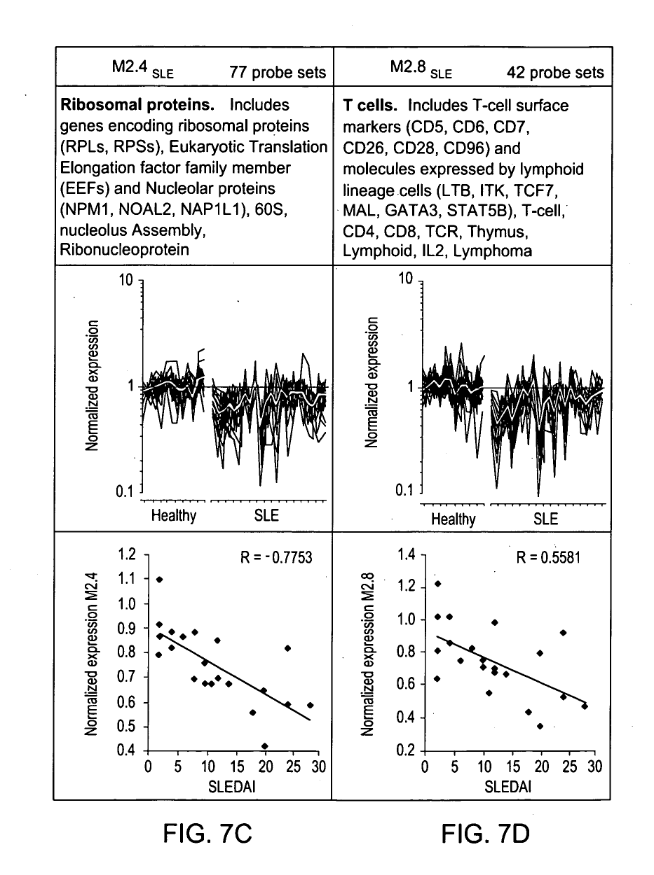

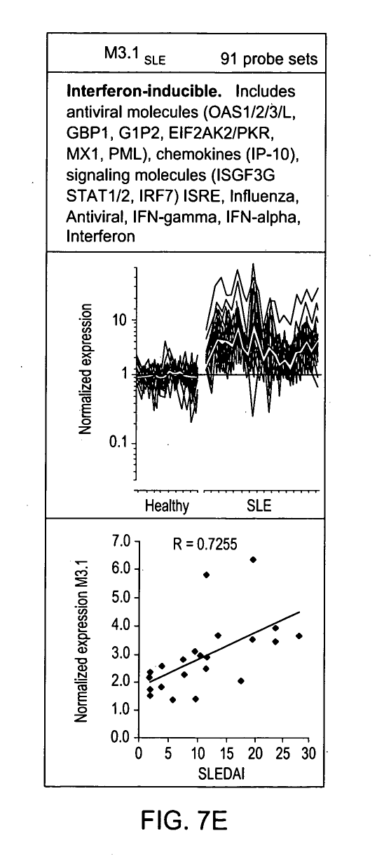

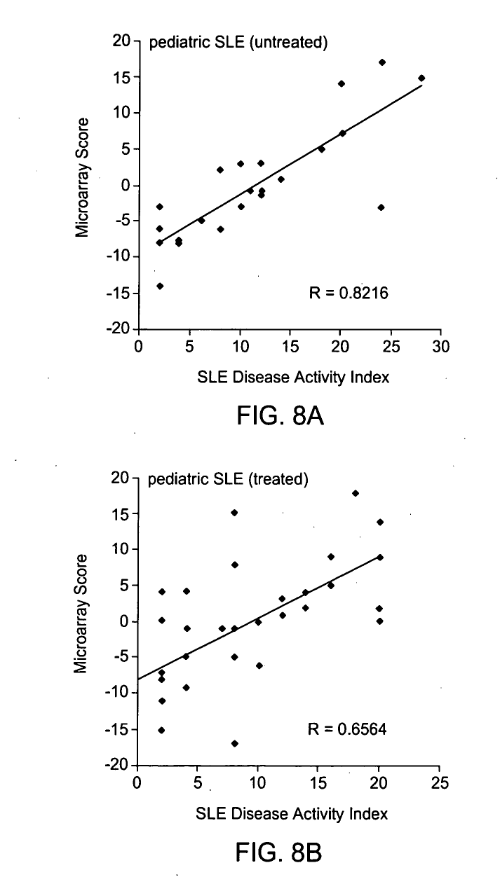

Definition of multivariate microarray transcriptional scores: The analysis of pediatric SLE patient profiles carried out above (FIG. 5) unequivocally linked transcriptional vectors and clinical disease manifestations. Also, correlated composite expression values were obtained for individual vectors and the clinical activity index (SLEDAI) computed for each of the patients in the untreated cohort. It was found that two of the transcriptional vectors correlated positively with disease activity (FIG. 7: M2.2 and M3.1, “neutrophil” and “interferon-inducible” modules, respectively), while three other vectors correlated negatively (FIG. 7: M1.7, M2.4 and M2.8, including transcripts associated with “ribosomal proteins” and “T-cells”). Decomposing microarray transcriptional data in distinct vectors permitted us to combine these five parameters into a single multivariate indicator. A novel non-parametric method for analyzing multivariate ordinal data was used to score the patients (described in detail in (Wittkowski et al., 2004). Microarray “U-scores” obtained for all patients in the untreated cohort were then correlated with SLEDAI (FIG. 8a; Spearman, R=0.82, p<0.0001). This group included one outlier (SLE 98) with a high SLEDAI and comparatively low microarray U-score. Interestingly this patient was the only one to carry two autoimmune diagnoses: SLE and hypothyroidism. Furthermore, this patient was diagnosed with SLE nephritis class IV but eventually failed to respond to conventional therapy with IV cyclophosphamide. Using the same five vectors, scores were generated for the treated, pediatric SLE patient cohort (n=31). Correlation between “transcriptional U-score” and disease activity index was once again strongly significant (FIG. 8b; Spearman correlation R=0.66, p<0.0001) (FIG. 4b).

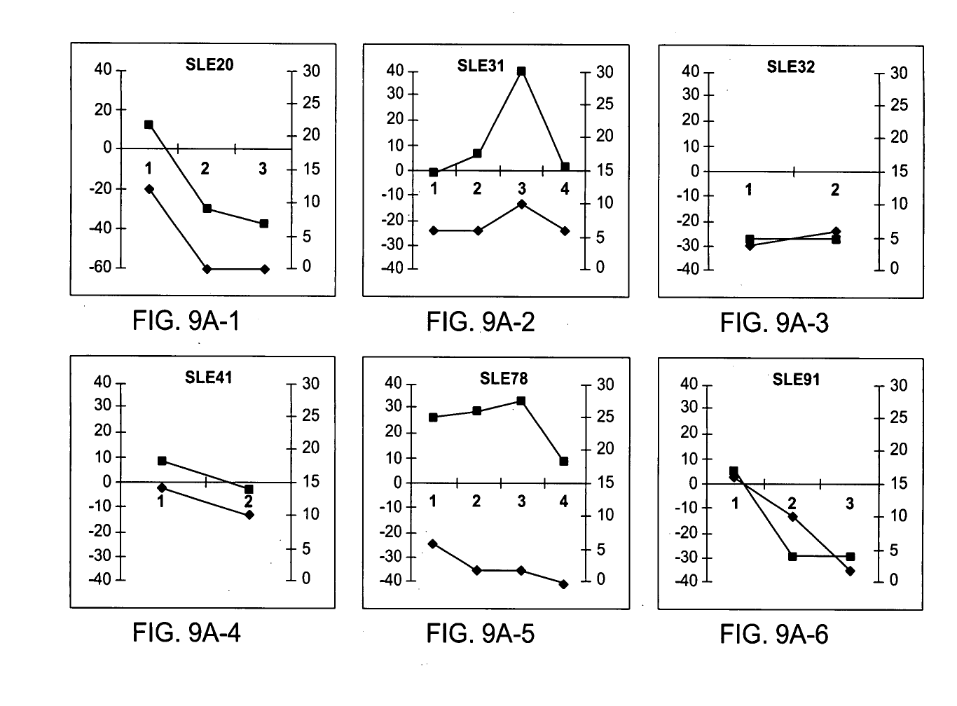

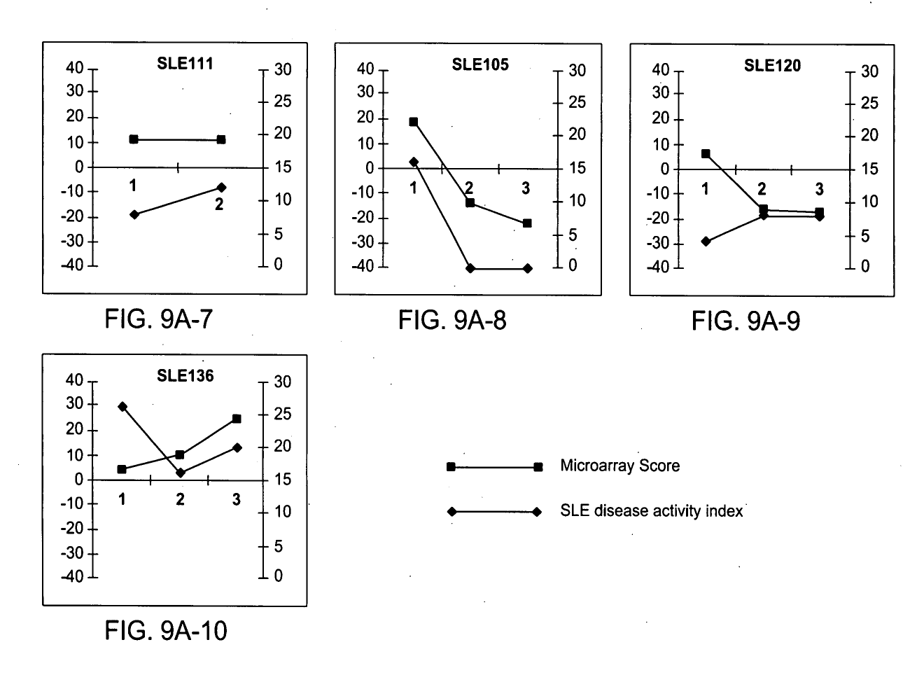

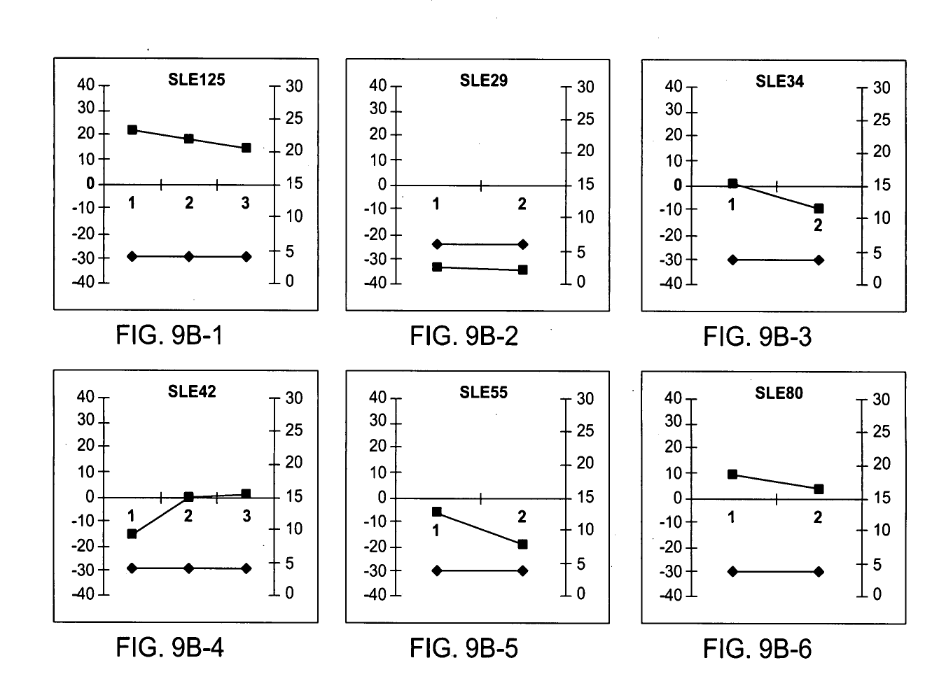

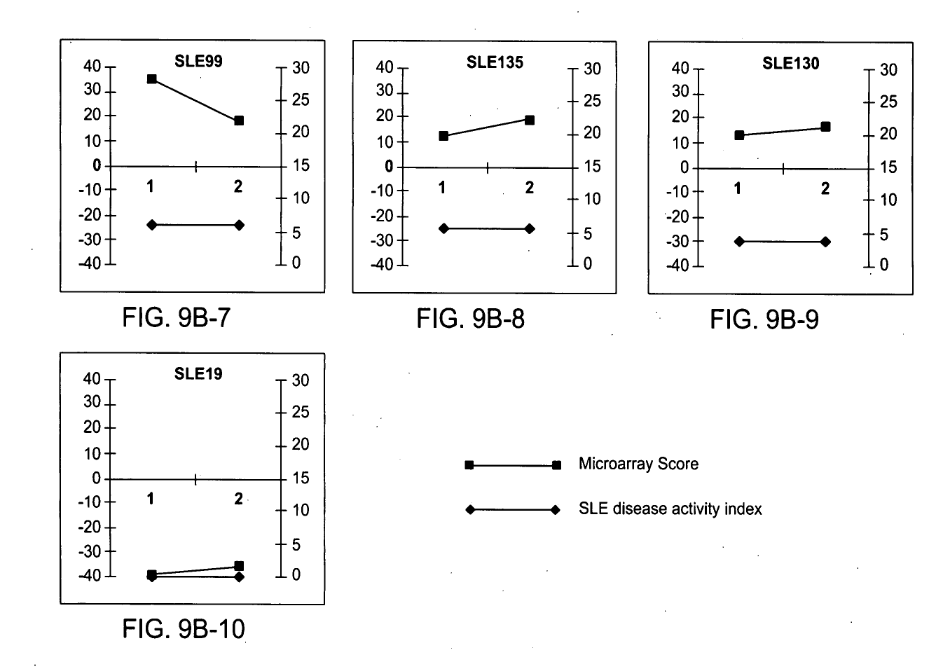

Longitudinal follow up of disease severity: Lupus disease flares which are associated with transient episodes of high morbidity can also lead to an irreversibly worsening of the status of the patient. The relevance of the microarray multivariate score described above was tested for the longitudinal monitoring of disease activity in Lupus patients. A cohort of 20 pediatric SLE patients was followed for disease activity over time. A transcriptome of microarray data was obtained from each of these patients at multiple time points (two to four time points, intervals between each time point varied from one month to 18 months). Microarray U scores were computed for these patients as described above. Half of the patients had been included in the cross-sectional analysis before they were enrolled in this longitudinal study. During the follow-up period, the SLEDAI fluctuated in 10 patients (FIG. 9a) while it remained constant in the other 10 (FIG. 9b). Parallel trends were observed between transcriptional U-scores and SLEDAI longitudinal measures in a majority of patients. Additionally, the overall SLEDAI index and microarray U-scores reflected similar activities according to their respective scales except in 6 patients (SLE31, SLE78, SLE125, SLE130, SLE135 and SLE 99) in whom the microarray U-scores were disproportionately high compared to SLEDAI scores. One of the patients with the highest discrepancy (SLE78) was diagnosed during the follow-up period with a life-threatening complication (pulmonary hypertension) which is not computed within the SLEDAI. The U-score, therefore, reflected better the overall disease activity of this patient. In addition, disease flaring and subsequent recovery was detected in one patient (SLE31) upon longitudinal follow up of both SLEDAI and microarray score. Interestingly, however, the amplitude of change observed in the case of the microarray U-score appears not only to be much greater (0 to 40 vs. 6 to 10 for SLEDAI), but an increase could already be detected at the second time point, 2 months before the worsening of the clinical condition of this patient could be detected by SLEDAI. Thus, these data illustrate the potential value of microarray disease activity scores for the longitudinal follow up of disease activity in individual SLE patients.

Modular transcriptional data are reproducible across microarray platforms. To be truly viable as diagnostic indicators immune transcriptional vectors must prove reliable. Early on, poor reproducibility of microarray results obtained by different laboratories and across platforms has raised suspicion about the validity of these results and remains a major concern, especially in a clinical setting (Bammler et al., 2005; loannidis, 2005; Irizarry et al., 2005; Larkin et al., 2005; Michiels et al., 2005). Modular transcriptional profiles were obtained and compared using two commercial microarray platforms, Affymetrix and Illumina. PBMCs were isolated from four healthy volunteers and ten liver transplant recipients. Starting from the same source of total RNA, targets were generated independently and analyzed using Affymetrix U133 GeneChips (at the Baylor Institute for Immunology Research) and Illumina Human Ref8 BeadChips (at Illumina Inc.). Fundamental differences exist between the two microarray technologies (see Methods for details). Probe IDs provided by each manufacturer were converted into a common ID that was used for matching gene expression profiles. When directly compared, gene expression levels generated by the Affymetrix and Illumina platforms correlated poorly (Pearson correlation between gene expression levels measured by Affymetrix and Illumina platforms for the different samples: R² median (range)=0.13 (0.02-0.5) for genes forming M1.2; 0.36 (0.17-0.55) for genes forming M3.1; and 0.19 (0.06-0.4) for genes forming M3.2). These results are in line with the findings of published microarray cross-platform comparison studies (Bammler et al., 2005; Irizarry et al., 2005; Jarvinen et al., 2004; Larkin et al., 2005; Tan et al., 2003).

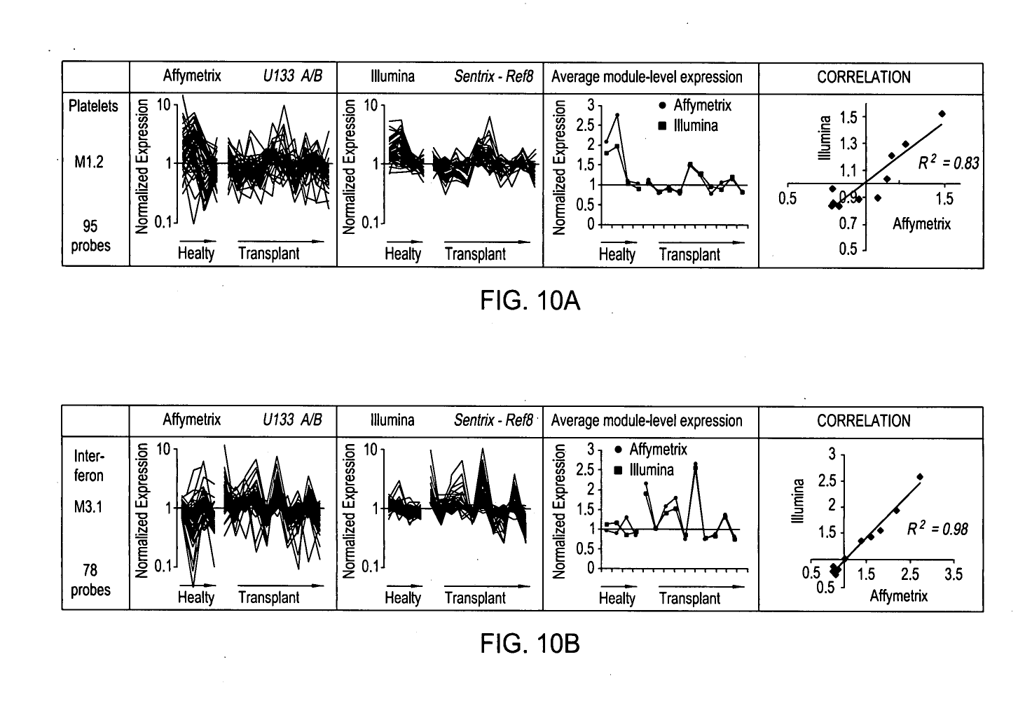

Expression profiles obtained for shared sets of genes are shown in FIG. 10 for modules M1.2 (“platelets”), M3.1 (“interferon”) and M3.2 (“inflammation”). Interestingly, for each module, changes in gene expression across samples measured by the Illumina system appeared tightly coordinated. This finding is particularly meaningful since the initial selection of sets of co-expressed genes (transcriptional modules) was exclusively based on gene expression data generated using Affymetrix GeneChips. Next, a unique expression value recapitulating transcriptional change at the module-level (see FIG. 4, step III) was derived. Modular expression levels generated by Affymetrix and Illumina platforms were highly comparable (FIG. 10; transplant group Pearson correlation coefficient R²=0.83, 0.98 and 0.93, for M1.2, M3.1 and M3.2 respectively; p<0.0001). Taken together, these results demonstrate that modular transcriptional data can be reproduced across microarray platforms.

Microarray data are prone to noise and as a result can be difficult to exploit (Michiels et al., 2005; Tuma, 2005). Indeed, carrying out group comparisons for thousands of transcripts will produce datasets containing significant proportions of noise (false positive results) that may lead to spurious discoveries (Ioannidis, 2005; Tuma, 2005). In order to address this fundamental issue, a preliminary step including the extraction of sets of coordinately expressed transcripts (i.e. transcriptional modules) from an extensive microarray data collection generated in the context of a wide range of diseases was used. Modules were formed from groups of transcripts following the same complex expression pattern across hundreds of samples and are therefore likely to be biologically related. The advantage was confirmed by an analysis of the literature associated with the genes forming each module (FIG. 2c). In summary, the modular decomposition of microarray transcriptional data permits to focus the analysis on well defined groups of coordinately expressed genes that contain reduced amounts of noise and carry identifiable biological meaning. This data mining strategy is applicable in a larger context, e.g., in other biological systems (other tissues, tumor samples as well as primary cells or cell lines) and for other types of data (e.g., proteomics).

Novel approaches for exploiting data acquired on a global level are required in order to translate the technological advances of the “omics revolution” into mainstream health care (Bilello, 2005; Weston and Hood, 2004). The development of immune transcriptional vectors may be an important step towards reaching this goal. It is illustrated herein that the potential clinical applications derived from this approach in two areas: (1) the identification of mechanisms of pathogenesis, and (2) the discovery of disease biomarkers.

Gene expression profiling can provide invaluable insights into molecular mechanisms underpinning disease processes (Bennett et al., 2003; Pascual et al., 2005), but the presence of noise and the scale of microarray datasets can hinder biological interpretation (Ioannidis, 2005). Decomposing transcriptional profiles in a set of well characterized modules provides a conceptual framework that facilitates the elucidation of these data. The representation of transcriptional changes on “module maps” (FIG. 2b) is particularly conductive to comparative analyses carried out across diseases, especially in the study of a universal tissue such as blood. I was observed that transcripts belonging to module M1.4 were overexpressed preferentially in patients with melanoma and in liver transplant recipients, subsequently confirming this finding using an alternative approach (analysis of significance patterns). These transcripts included-inhibitors of interleukin-2 transcription, inhibitors of NF-kappaB and MAPK pathways as well as molecules able to block cell proliferation. These findings point toward a functional convergence between immunosuppressive mechanisms operating in patients with advanced melanoma and pharmacologically-treated transplant recipients. The fact that the transcripts specifically induced in immunosuppressed patients also include glucocorticoid-inducible genes (e.g., DSIPI, CXCR⁴, JUN) and hormone nuclear receptors thought to play key roles in the development and effector functions of T lymphocytes (NR⁴A2 and RORA) (Winoto and Littman, 2002) suggest a possible role for steroid hormones in melanoma-mediated immunosuppression.

Immune transcriptional vectors represent a novel class of disease biomarkers. A direct extension of the modular data mining strategy described herein is the use of expression vectors to capture the global changes observed both at the module- and gene-level. It was found that diseases could be characterized by a unique combination of modular changes. In addition to changes observed at the module-level (first round of selection), vectors also reflect differences that can be observed at the gene-level (second round of selection). As a result, sets of transcriptional vectors are highly disease specific. Remarkably, for each patient a set of “vectorial profiles” could potentially be obtained for any number of diseases based on the same data acquired on a global scale. Averaged transcriptional values derived for each vector proved remarkably robust, as indicated by the excellent reproducibility obtained across microarray platforms and laboratories. This finding is particularly meaningful since the identification of reliable transcriptional markers constitutes an important step towards the development of mainstream applications for microarray technologies in clinical settings.

Processing of blood samples: Blood samples were collected in acid citrate dextrose or EDTA tubes (BD Vacutainer) and immediately delivered at room temperature to the Baylor Institute for Immunology Research, Dallas, Tex., for processing. Peripheral blood mononuclear cells (PBMCs) were isolated via Ficoll gradient and immediately lysed in RLT reagent (Qiagen, Valencia, Calif.) with beta-mercaptoethanol (BME) and stored at −80° C. prior to the RNA extraction step.

Microarray analysis: Total RNA was isolated using the RNeasy kit (Qiagen) according to the manufacturer's instructions and RNA integrity assessed using an Agilent 2100 Bioanalyzer (Agilent, Palo Alto, Calif.).

Affymetrix GeneChips: These microarrays consist of short oligonucleotide probe sets synthesized in situ on a quartz wafer. Target labeling was performed according to the manufacturer's standard protocol (Affymetrix Inc., Santa Clara, Calif.). Biotinylated cRNA targets were purified and subsequently hybridized to Affymetrix HG-U133A and U133B GeneChips (>44,000 probe sets). Arrays were scanned using an Affymetrix confocal laser scanner. Microarray Suite, Version 5.0 (MAS 5.0; Affymetrix) software was used to assess fluorescent hybridization signals, to normalize signals, and to evaluate signal detection calls. Normalization of signal values per chip was achieved using the MAS 5.0 global method of scaling to the target intensity value of 500 per GeneChip. A gene expression analysis software program, GeneSpring, Version 7.1 (Agilent), was used to perform statistical analysis and clustering.

Illumina BeadChips: These microarrays consist of 50mer oligonucleotide probes attached to 3 μm beads, which are lodged into microwells at the surface of a glass slide. Samples were processed and data acquired by Illumina Inc. (San Diego, Calif.). Targets were prepared using the Illumina RNA amplification kit (Ambion, Austin, Tex.). cRNA targets were hybridized to Sentrix HumanRef8 BeadChips (>25,000 probes), which were scanned on an Illumina BeadStation 500. Illumina's Beadstudio software was used to assess fluorescent hybridization signals.

Module extraction algorithm: Sets of coordinately regulated genes, or transcriptional modules, were extracted from a leukocyte microarray dataset using a custom mining algorithm (FIG. 1b: Step I and FIG. 1c). Gene expression profiles from a total of 239 PBMC samples generated using Affymetrix U133A and U133B GeneChips (>44,000 probe sets) were obtained for eight groups of patients (with systemic juvenile idiopathic arthritis, systemic lupus erythematosus, type I diabetes, metastatic melanoma, acute infections—Escherichia coli, Staphylococcus aureus and influenza A—and liver transplant recipients). For each group, transcripts that were present in at least 50% of all conditions were segregated into 30 clusters (k-means clustering: clusters C1 through C30). The cluster assignment for each gene was recorded in a table and distribution patterns were compared among all the genes. Modules were selected using an iterative process, starting with the largest set of genes that belonged to the same cluster in all study groups (i.e. genes that were found in the same cluster in eight of the eight experimental groups). The selection was then expanded from this core reference pattern to include genes with 7/8, 6/8 and 5/8 matches. The resulting set of genes formed a transcriptional module and was withdrawn from the selection pool. The process was repeated starting with the second largest group of genes, progressively reducing the level of stringency.

U-scores: The detailed explanation of this method has been published recently (Wittkowski et al., 2004) and the required tools are available at http://Mustat.Rockefeller.edu. Briefly, scores were obtained by computing the average normalized expression levels for all transcripts within the modules that were identified as differentially expressed in SLE PBMCs.

Literature profiling: The literature profiling algorithm employed in this study has been previously described in detail (Chaussabel and Sher, 2002). This approach links genes sharing similar keywords. It uses hierarchical clustering to analyze patterns of term occurrence in literature abstracts.

Biomarker discovery plays a critical role in the development of novel diagnostics and therapies (Ratner, 2005), and while microarray data constitute a very attractive source of candidate markers, very little progress has been made towards the development of applications at the bedside. Indeed, markers derived from microarray analyses have been difficult to validate and proved to be unstable (Frantz, 2005; Michiels et al., 2005). The use of modular data mining strategy and composite expression vectors were found to be consistent with the global changes observed at the module and gene-level. Using modules as a foundation grounds expression vectors to coherent functional and transcriptional units containing minimized amounts of noise. The fact that vectors are composite (i.e. formed by a combination of transcripts) further contributes to the stability of these markers. Indeed, vector expression values proved remarkably robust, as indicated by the high reproducibility obtained across microarray platforms (FIG. 10); as well as the validation results obtained in an independent set of pediatric lupus patients (FIG. 5d). More importantly these data and studies demonstrate that composite expression vectors can be directly linked to clinical disease activity (e.g., in patients with lupus; FIGS. 7 to 10). These improve the reliability of microarray data, which is a prerequisite for the widespread use of this technology in clinical practice (Shi, 2006).

The biomarker discovery strategy that we have developed is particularly well adapted for the exploitation of data acquired on a global scale. Starting from ˜44,000 transcripts we have defined 28 modules composed of nearly 5000 transcripts. Sets of composite vectors were then formed through two selection rounds carried out at the module- and gene-level. This precise tailoring permits to optimize the performance of a given set of markers by increasing its specificity. Finally, vectors can in turn be combined to obtain unique multivariate scores, therefore delivering results in a form that is compatible with mainstream clinical practice. Interestingly, multivariate scores recapitulate global patterns of change rather than changes in individual markers. The development of such “global biomarkers” constitutes a promising prospect for both diagnostic and pharmacogenomics fields.

In conclusion, expression vectors belong to a novel class of biomarkers capable of leveraging data acquired on a global scale. The clinical relevance of this approach for the diagnosis and assessment of disease progression in patients with systemic lupus is demonstrated herein. As illustrated by our results, composite expression vectors could also be useful indicators for the evaluation of the efficacy, safety, and mechanism of action of novel drugs. Other potential applications include disease prognosis and health monitoring.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. Carroll, M. C. 2004. A protective role for innate immunity in systemic lupus erythematosus. Nat Rev Immunol 4:825-831.
2. Manderson, A. P., Botto, M., and Walport, M. J. 2004. The role of complement in the development of systemic lupus erythematosus. Annu Rev Immunol 22:431-456.
3. Manzi, S., Ahearn, J. M., and Salmon, J. 2004. New insights into complement: a mediator of injury and marker of disease activity in systemic lupus erythematosus. Lupus 13:298-303.
4. Nambiar, M. P., Juang, Y. T., Krishnan, S., and Tsokos, G. C. 2004. Dissecting the molecular mechanisms of TCR zeta chain downregulation and T cell signaling abnormalities in human systemic lupus erythematosus. Int Rev Immunol 23:245-263.
5. Kong, P. L., Odegard, J. M., Bouzahzah, F., Choi, J. Y., Eardley, L. D., Zielinski, C. E., and Craft, J. E. 2003. Intrinsic T cell defects in systemic autoimmunity. Ann N Y Acad Sci 987:60-67.
6. Grammer, A. C., and Lipsky, P. E. 2003. B cell abnormalities in systemic lupus erythematosus. Arthritis Res Ther 5 Suppl 4:822-27.
7. Jorgensen, T. N., Gubbels, M. R., and Kotzin, B. L. 2003. Links between type I interferons and the genetic basis of disease in mouse lupus. Autoimmunity 36:491-502.
8. Blanco, P., Palucka, A. K., Gill, M., Pascual, V., and Banchereau, J. 2001. Induction of dendritic cell differentiation by IFN-alpha in systemic lupus erythematosus. Science 294:1540-1543.
9. Santiago-Raber, M. L., Baccala, R., Haraldsson, K. M., Choubey, D., Stewart, T. A., Kono, D. H., and Theofilopoulos, A. N. 2003. Type-I interferon receptor deficiency reduces lupus-like disease in NZB mice. J Exp Med 197:777-788.
10. Bencivelli, W., Vitali, C., Isenberg, D. A., Smolen, J. S., Snaith, M. L., Sciuto, M., and Bombardieri, S. 1992. Disease activity in systemic lupus erythematosus: report of the Consensus Study Group of the European Workshop for Rheumatology Research. III. Development of a computerised clinical chart and its application to the comparison of different indices of disease activity. The European Consensus Study Group for Disease Activity in SLE. Clin Exp Rheumatol 10:549-554.
11. Hay, E. M., Bacon, P. A., Gordon, C., Isenberg, D. A., Maddison, P., Snaith, M. L., Symmons, D. P., Viner, N., and Zoma, A. 1993. The BILAG index: a reliable and valid instrument for measuring clinical disease activity in systemic lupus erythematosus. Q J Med 86:447-458.
12. Bombardier, C., Gladman, D. D., Urowitz, M. B., Caron, D., and Chang, C. H.
1992. Derivation of the SLEDAI. A disease activity index for lupus patients. The Committee on Prognosis Studies in SLE. Arthritis Rheum 35:630-640.
13. Liang, M. H., Socher, S. A., Larson, M. G., and Schur, P. H. 1989. Reliability and validity of six systems for the clinical assessment of disease activity in systemic lupus erythematosus. Arthritis Rheum 32:1107-1118.
14. Bae, S. C., Koh, H. K., Chang, D. K., Kim, M. H., Park, J. K., and Kim, S. Y. 2001. Reliability and validity of systemic lupus activity measure-revised (SLAM-R) for measuring clinical disease activity in systemic lupus erythematosus. Lupus 10:405-409.
15. Petri, M., Buyon, J., and Kim, M. 1999. Classification and definition of major flares in SLE clinical trials. Lupus 8:685-691.
16. Jimenez, S., Cervera, R., Font, J., and Ingelmo, M. 2003. The epidemiology of systemic lupus erythematosus. Clin Rev Allergy Immunol 25:3-12.
17. Rood, M. J., ten Cate, R., van Suijlekom-Smit, L. W., den Ouden, E. J., Ouwerkerk, F. E., Breedveld, F. C., and Huizing a, T. W. 1999. Childhood-onset Systemic Lupus Erythematosus: clinical presentation and prognosis in 31 patients. Scand J Rheumatol 28:222-226.
18. Brunner, H. I., Silverman, E. D., To, T., Bombardier, C., and Feldman, B. M. 2002. Risk factors for damage in childhood-onset systemic lupus erythematosus: cumulative disease activity and medication use predict disease damage. Arthritis Rheum 46:436-444.
19. Tan, E. M., Cohen, A. S., Fries, J. F., Masi, A. T., McShane, D. J., Rothfield, N. F., Schaller, J. G., Talal, N., and Winchester, R. J. 1982. The 1982 revised criteria for the classification of systemic lupus erythematosus. Arthritis Rheum 25:1271-1277.
20. Hochberg, M. C. 1997. Updating the American College of Rheumatology revised criteria for the classification of systemic lupus erythematosus. Arthritis Rheum 40:1725.
21. Tan, E. M., Feltkamp, T. E., Smolen, J. S., Butcher, B., Dawkins, R., Fritzler, M. J., Gordon, T., Hardin, J. A., Kalden, J. R., Lahita, R. G., et al. 1997. Range of antinuclear antibodies in “healthy” individuals. Arthritis Rheum 40:1601-1611.
22. Al-Allaf, A. W., Ottewell, L., and Pullar, T. 2002. The prevalence and significance of positive antinuclear antibodies in patients with fibromyalgia syndrome: 2-4 years' follow-up. Clin Rheumatol 21:472-477.
23. Staud, R. 2004. Fibromyalgia pain: do we know the source? Curr Opin Rheumatol 16:157-163.
24. Bennett, L., Palucka, A. K., Arce, E., Cantrell, V., Borvak, J., Banchereau, J., and Pascual, V. 2003. Interferon and granulopoiesis signatures in systemic lupus erythematosus blood. J Exp Med 197:711-723.
25. Baechler, E. C., Batliwalla, F. M., Karypis, G., Gaffney, P. M., Ortmann, W. A., Espe, K. J., Shark, K. B., Grande, W. J., Hughes, K. M., Kapur, V., et al. 2003. Interferon-inducible gene expression signature in peripheral blood cells of patients with severe lupus. Proc Natl Acad Sci USA 100:2610-2615.
26. Crow, M. K., Kirou, K. A., and Wohlgemuth, J. 2003. Microarray analysis of interferon-regulated genes in SLE. Autoimmunity 36:481-490.
27. Kirou, K. A., Lee, C., George, S., Louca, K., Papagiannis, I. G., Peterson, M. G., Ly, N., Woodward, R. N., Fry, K. E., Lau, A. Y., et al. 2004. Coordinate overexpression of interferon-alpha-induced genes in systemic lupus erythematosus. Arthritis Rheum 50:3958-3967.
28. Ito, T., Amakawa, R., Inaba, M., Ikehara, S., Inaba, K., and Fukuhara, S. 2001. Differential regulation of human blood dendritic cell subsets by IFNs. J Immunol 166:2961-2969.
29. Santini, S. M., Lapenta, C., Logozzi, M., Parlato, S., Spada, M., Di Pucchio, T., and Belardelli, F. 2000. Type I interferon as a powerful adjuvant for monocyte-derived dendritic cell development and activity in vitro and in Hu-PBL-SCID mice. J Exp Med 191:1777-1788.
30. Arce, E., Jackson, D. G., Gill, M. A., Bennett, L. B., Banchereau, J., and Pascual, V. 2001. Increased frequency of pre-germinal center B cells and plasma cell precursors in the blood of children with systemic lupus erythematosus. J Immunol 167:2361-2369.
31. Jego, G., Bataille, R., and Pellat-Deceunynck, C. 2001. Interleukin-6 is a growth factor for nonmalignant human plasmablasts. Blood 97:1817-1822.
32. Odendahl, M., Jacobi, A., Hansen, A., Feist, E., Hiepe, F., Burmester, G. R., Lipsky, P. E., Radbruch, A., and Domer, T. 2000. Disturbed peripheral B lymphocyte homeostasis in systemic lupus erythematosus [In Process Citation]. J Immunol 165:5970-5979.
33. Shodell, M., Shah, K., and Siegal, F. P. 2003. Circulating human plasmacytoid dendritic cells are highly sensitive to corticosteroid administration. Lupus 12:222-230.
34. Gladman, D. D., Ibanez, D., and Urowitz, M. B. 2002. Systemic lupus erythematosus disease activity index 2000. J Rheumatol 29:288-291.
35. Tibshirani, R., Hastie, T., Narasimhan, B., and Chu, G. 2002. Diagnosis of multiple cancer types by shrunken centroids of gene expression. Proc Natl Acad Sci USA 99:6567-6572.
36. Wittkowski, K. M., Lee, E., Nussbaum, R., Chamian, F. N., and Krueger, J. G. 2004. Combining several ordinal measures in clinical studies. Stat Med 23:1579-1592.
37. Segal, E., Friedman, N., Kaminski, N., Regev, A., and Koller, D. 2005. From signatures to models: understanding cancer using microarrays. Nat Genet 37 Suppl:S38-45.
38. Choi, P., and Chen, C. 2005. Genetic expression profiles and biologic pathway alterations in head and neck squamous cell carcinoma. Cancer.
39. Thach, D. C., Agan, B. K., Olsen, C., Diao, J., Lin, B., Gomez, J., Jesse, M., Jenkins, M., Rowley, R., Hanson, E., et al. 2005. Surveillance of transcriptomes in basic military trainees with normal, febrile respiratory illness, and convalescent phenotypes. Genes Immun.
40. Kirou, K. A., Lee, C., George, S., Louca, K., Peterson, M. G., and Crow, M. K. 2005. Activation of the interferon-alpha pathway identifies a subgroup of systemic lupus erythematosus patients with distinct serologic features and active disease. Arthritis Rheum 52:1491-1503.
41. Wittkowski, K., Lee, E., Nussbaum, R., Chamian, F., and Krueger, J. G. 2004. Combining several ordinal measures in clinical studies. Statist Med 23.

What is claimed is:

1. A method for determining whether an individual has systemic lupus erythematosus (SLE), comprising:
obtaining the transcriptome of a patient;
scoring the transcriptome based on one or more transcriptional modules; and
determining the patient's disease or condition based on the presence, absence or level of expression of genes within the transcriptome in the one or more transcriptional modules that are indicative of SLE.

2. The method of claim 1, wherein the transcriptional modules is obtained by:
iteratively selecting gene expression values for one or more transcriptional modules by:
selecting for the module the genes from each cluster that match in every disease or condition;
removing the selected genes from the analysis; and
repeating the process of gene expression value selection for genes that cluster in a sub-fraction of the diseases or conditions; and
iteratively repeating the generation of modules for each cluster until all gene clusters are exhausted.

3. The method of claim 2, wherein the clusters are selected from expression value clusters, keyword clusters, metabolic clusters, disease clusters, infection clusters, transplantation clusters, signaling clusters, transcriptional clusters, replication clusters, cell-cycle clusters, siRNA clusters, miRNA clusters, mitochondrial clusters, T cell clusters, B cell clusters, cytokine clusters, lymphokine clusters, heat shock clusters and combinations thereof.

4. The method of claim 1, wherein the patient is a human SLE patient.

5. The method of claim 1, wherein the patient is provided with a therapeutically effective amount of a drug selected from the group consisting of: a glucocorticoid, a non-steroidal anti-inflammatory agent and an immunosuppressant.

6. A method of diagnosing or monitoring an autoimmune or chronic inflammatory disease in a patient, comprising detecting the expression level of two or more gene modules that include genes selected from: immunoglobulin, neutrophils, interferon, T cells, and ribosomal proteins.

7. The method of claim 6, wherein the one or more genes is selected from:
Transcriptional modules
M 1.7 one or more MHC/Ribosomal genes comprising MHC class I molecules: HLA-A,B,C,G,E)+Beta 2-microglobulin (B2M), Ribosomal proteins: RPLs, RPSs;
M 2.2 one or more Neutrophil genes comprising Lactotransferrin: LTF, defensin: DEAF1, Bacterial Permeability Increasing protein (BPI), Cathelicidin antimicrobial protein (CAMP);
M 2.4 one or more Ribosomal protein genes comprising RPLs, RPSs, Eukaryotic Translation Elongation factor family members (EEFs), Nucleolar proteins: NPM1, NOAL2, NAPIL1;
M 2.8 one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B; and
M 3.1 one or more interferon-inducible genes comprising antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G).

8. The method of claim 6, wherein the disease comprises systemic lupus erythematosus (SLE).

9. The method of claim 6, wherein the expression level is detected by measuring the RNA level expressed by the gene.

10. The method of claim 6, further comprising isolating RNA from the patient prior to detecting the RNA level expressed by the gene.

11. The method of claim 6, wherein the RNA level is detected by PCR, by hybridization or hybridization to an oligonucleotide.

12. The method of claim 6, wherein the modules analyzed further comprise the genes listed herein in modules listed as M 1.1, M 1.7, M 2.1, M 2.2, M 2.3, M 2.4, M 2.5, M 2.6, M 2.7, M 2.8 and/or M 3.1.

13. The method of claim 6, wherein the modules analyzed comprise one or more genes from each of the following:
a first module includes one or more of the following genes or gene fragments: Hs.406683; Hs.514581; Hs.546356; Hs.374553; Hs.448226; Hs.381172; Hs.534255; Hs.406620; Hs.534255; Hs.410817; Hs.136905; Hs.546394; Hs.419463; Hs.5308; Hs.514581; Hs.387804; Hs.546286; Hs.300141; Hs.356366; Hs.433427; Hs.533624; Hs.546356; Hs.370504; Hs.433701; Hs.153177; Hs.150580; Hs.514581; Hs.356794; Hs.419463; Hs.433427; Hs.469473; Hs.380953; Hs.410817; Hs.421257; Hs.408054; Hs.433529; Hs.458476; Hs.439552; Hs.156367; Hs.546291; Hs.546290; Hs.514581; Hs.144835; Hs.439552; Hs.356502; Hs.397609; Hs.446628; Hs.546356; Hs.265174; Hs.425125; Hs.374596; Hs.381126; Hs.381061; Hs.406620; Hs.533977; Hs.447600; Hs.148340; Hs.421907; Hs.448226; Hs.410817; Hs.119598; Hs.433427; Hs.410817; Hs.8102; Hs.446628; Hs.356572; Hs.381123; Hs.515329; Hs.408054; Hs.483877; Hs.386384; Hs.337766; Hs.408073; Hs.546289; Hs.374596; Hs.512199; Hs.119598; Hs.499839; Hs.446588; Hs.356572; Hs.397609; Hs.356572; Hs.144835; Hs.515329; Hs.534833; Hs.374588; Hs.144835; Hs.80545; Hs.546356; Hs.400295; Hs.119598; Hs.408073; Hs.412370; Hs.401929; Hs.425125; Hs.374588; Hs.374588; Hs.356366; Hs.186350; and Hs.186350; and;
a second module includes one or more of the following genes or gene fragments: Hs.513711; Hs.375108; Hs.176626; Hs.2962; Hs.41; Hs.99863; Hs.530049; Hs.51120; Hs.480042; Hs.36977; Hs.294176; Hs.529019; Hs.2582; Hs.550853; Hs.529517; Hs.204238; and;
a third module includes one or more of the following genes or gene fragments: Hs.518827; Hs.8102; Hs.190968; Hs.508266; Hs.523913; Hs.437594; Hs.515598; Hs.54780; Hs.534384; Hs.527105; Hs.522885; Hs.462341; Hs.127610; Hs.408018; Hs.381219; Hs.6917; Hs.109798; Hs.497581; Hs.369728; Hs.432485; Hs.314359; Hs.409140; Hs.529798; Hs.477028; Hs.107003; Hs.528668; Hs.314359; Hs.6917; Hs.333120; Hs.500822; Hs.131255; Hs.469925; Hs.410817; Hs.277517; Hs.529631; Hs.367900; Hs.408054; Hs.467284; Hs.111099; Hs.378103; Hs.108332; Hs.397609; Hs.80545; Hs.529631; Hs.472558; Hs.519452; Hs.516023; Hs.438429; Hs.515472; Hs.512675; Hs.438429; Hs.314359; Hs.75056; Hs.482526; Hs.333388; Hs.483305; Hs.515329; Hs.288856; Hs.546288; Hs.483305; Hs.534346; Hs.528435; Hs.381219; Hs.469925; Hs.172791; Hs.190968; Hs.182825; Hs.492599; Hs.406620; Hs.549130; Hs.532359; Hs.534346; Hs.421257; Hs.511831; Hs.380920; Hs.311640; Hs.546356; Hs.119598; Hs.405590; Hs.178551; Hs.499839; Hs.148340; Hs.483305; Hs.505735; Hs.381219; Hs.299002; Hs.532359; Hs.5662; Hs.515329; Hs.408073; Hs.515070; Hs.448226; Hs.515329; Hs.511582; Hs.421608; Hs.186350; Hs.529798; and Hs.294094; and;
a fourth module includes one or more of the following genes or gene fragments: Hs.397891; Hs.438801; Hs.125036; Hs.210891; Hs.220629; Hs.376208; Hs.316931; Hs.196981; Hs.271272; Hs.397891; Hs.7946; Hs.505326; Hs.369581; Hs.58685; Hs.7236; Hs.17109; Hs.49143; Hs.505806; Hs.60339; Hs.13262; Hs.22380; Hs.233044; Hs.133397; Hs.445489; Hs.60339; Hs.428214; Hs.431498; Hs.533994; Hs.533994; Hs.498317; Hs.533994; Hs.517717; Hs.173135; Hs.522679; Hs.446149; Hs.525700; Hs.519580; Hs.481704; Hs.379414; Hs.125036; Hs.440776; Hs.475602; Hs.173135; Hs.481704; Hs.167087; Hs. 142023; Hs.524134; Hs.98309; Hs.433700; Hs.480837; Hs.5019; Hs.525700; Hs.94229; Hs.446149; Hs.502710; and
a fifth module includes one or more of the following genes or gene fragments: Hs.276925; Hs.98259; Hs.478275; Hs.273330; Hs.175120; Hs.190622; Hs.175120; Hs.415534; Hs.62661; Hs.344812; Hs.145150; Hs.5148; Hs.302123; Hs.65641; Hs.62661; Hs.86724; Hs.120323; Hs.370515; Hs.291000; Hs.62661; Hs.118110; Hs.131431; Hs.464419; Hs.65641; Hs.145150; Hs.415534; Hs.54483; Hs.520162; Hs.414579; Hs.190622; Hs.374950; Hs.478275; Hs.369039; Hs.229988; Hs.458414; Hs.425777; Hs.531314; Hs.352018; Hs.526464; Hs.470943; Hs.514535; Hs.487933; Hs.481143; Hs.217484; Hs.524117; Hs.137007; Hs.458414; Hs.374650; Hs.470943; Hs.50842; Hs. 118633; Hs.130759; Hs.384598; Hs.524760; Hs.441975; Hs.530595; Hs.546467; Hs.529317; Hs.175687; Hs.112420; Hs.1706; Hs.523847; Hs.388733; Hs.163173; Hs.470943; Hs.481141; Hs.171426; Hs.174195; Hs.518201; Hs.118633; Hs.489118; Hs.489118; Hs.193842; Hs.551516; Hs.518203; Hs.371794; Hs.529317; Hs.195642; Hs.12341; Hs.414332; Hs.524760; Hs.479264; Hs.501778; Hs.414332; Hs.12646; Hs.518200; Hs.441975; Hs.441975; Hs.437609; Hs.130759; Hs.82316; Hs.518200; Hs.458485; Hs.31869; Hs.166120; Hs.549041; Hs.17518; Hs.546467; Hs.517307; Hs.549041; Hs.528634; Hs.389724; Hs.546523; Hs.82316; Hs.7155; Hs.521903; Hs.26663; Hs.120323; and Hs.926.

14. The method of claim 6, wherein the nucleotide sequence comprises DNA, RNA, cDNA, PNA, genomic DNA, or synthetic oligonucleotides.

15. The method of claim 6, wherein the expression is detecting by measuring protein levels of the gene.

16. A disease analysis tool comprising:
one or more gene probes selected from the group consisting of:
one or more MHC/Ribosomal genes comprising MHC class I molecules: HLA-A,B,C,G,E)+Beta 2-microglobulin (B2M), Ribosomal proteins: RPLs, RPSs;
one or more Neutrophil genes comprising Lactotransferrin: LTF, defensin: DEAF1, Bacterial Permeability Increasing protein (BPI), Cathelicidin antimicrobial protein (CAMP);
one or more Ribosomal protein genes comprising RPLs, RPSs, Eukaryotic Translation Elongation factor family members (EEFs), Nucleolar proteins: NPM1, NOAL2, NAP1L1;
one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B; and
one or more interferon-inducible genes comprising antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G).
sufficient to distinguish between an autoimmune disease, a viral infection a bacterial infection, cancer and transplant rejection.

17. A prognostic gene array comprising:
a customized gene array that comprises a combination of genes that are representative of one or more transcriptional modules, wherein the transcriptome of a patient that is contacted with the customized gene array is prognostic of SLE.

18. The array of claim 17, wherein the patient's response to therapy for SLE is monitored.

19. The array of claim 17, wherein the array can distinguish between an autoimmune disease, a viral infection a bacterial infection, cancer and transplant rejection.

20. The array of claim 17, wherein the array is organized into two or more transcriptional modules.

21. The array of claim 17, wherein the array is organized into three or more transcriptional modules comprising one or more submodules are selected from:

| | Number | | |
|---|---|---|---|
| | of probe | | |
| Submodule | sets | Keyword selection | Assessment |
| M 1.1 | 69 | Ig, Immunoglobulin, | Plasma cells. Includes genes coding for |
| | | Bone, Marrow, PreB, | Immunoglobulin chains (e.g. IGHM, IGJ, |
| | | IgM, Mu. | IGLL1, IGKC, IGHD) and the plasma cell |
| | | | marker CD38; |
| M 1.2 | 96 | Platelet, Adhesion, | Platelets. Includes genes coding for platelet |
| | | Aggregation, | glycoproteins (ITGA2B, ITGB3, GP6, GP1A/B), |
| | | Endothelial, Vascular | and platelet-derived immune mediators such as |
| | | | PPPB (pro-platelet basic protein) and PF4 |
| | | | (platelet factor 4); |
| M 1.3 | 47 | Immunoreceptor, | B-cells. Includes genes coding for B-cell surface |
| | | BCR, B-cell, IgG | markers (CD72, CD79A/B, CD19, CD22) and |
| | | | other B-cell associated molecules: Early B-cell |
| | | | factor (EBF), B-cell linker (BLNK) and B |
| | | | lymphoid tyrosine kinase (BLK); |
| M 1.4 | 87 | Replication, | Undetermined. This set includes regulators and |
| | | Repression, Repair, | targets of cAMP signaling pathway (JUND, |
| | | CREB, Lymphoid, | ATF4, CREM, PDE4, NR4A2, VIL2), as well as |
| | | TNF-alpha | repressors of TNF-alpha mediated NF-KB |
| | | | activation (CYLD, ASK, TNFAIP3); |
| M 1.5 | 130 | Monocytes, | Myeloid lineage. Includes molecules expressed |
| | | Dendritic, MHC, | by cells of the myeloid lineage (CD86, CD163, |
| | | Costimulatory, | FCGR2A), some of which being involved in |
| | | TLR4, MYD88 | pathogen recognition (CD14, TLR2, MYD88). |
| | | | This set also includes TNF family members |
| | | | (TNFR2, BAFF); |
| M 1.6 | 28 | Zinc, Finger, P53, | Undetermined. This set includes genes coding |
| | | RAS | for signaling molecules, e.g. the zinc finger |
| | | | containing inhibitor of activated STAT (PIAS1 |
| | | | and PIAS2), or the nuclear factor of activated T- |
| | | | cells NFATC3; |
| M 1.7 | 127 | Ribosome, | MHC/Ribosomal proteins. Almost exclusively |
| | | Translational, 40S, | formed by genes coding MHC class I molecules |
| | | 60S, HLA | (HLA-A, B, C, G, E) + Beta 2-microglobulin (B2M) |
| | | | or Ribosomal proteins (RPLs, RPSs); |
| M 1.8 | 86 | Metabolism, | Undetermined. Includes genes encoding |
| | | Biosynthesis, | metabolic enzymes (GLS, NSF1, NAT1) and |
| | | Replication, Helicase | factors involved in DNA replication (PURA, |
| | | | TERF2, EIF2S1); |
| M 2.1 | 72 | NK, Killer, Cytolytic, | Cytotoxic cells. Includes cytotoxic T-cells and |
| | | CD8, Cell-mediated, | NK-cells surface markers (CD8A, CD2, CD160, |
| | | T-cell, CTL, IFN-g | NKG7, KLRs), cytolytic molecules (granzyme, |
| | | | perforin, granulysin), chemokines (CCL5, |
| | | | XCL1) and CTL/NK-cell associated molecules |
| | | | (CTSW); |
| M 2.2 | 44 | Granulocytes, | Neutrophils. This set includes innate molecules |
| | | Neutrophils, Defense, | that are found in neutrophil granules |
| | | Myeloid, Marrow | (Lactotransferrin: LTF, defensin: DEAF1, |
| | | | Bacterial Permeability Increasing protein: BPI, |
| | | | Cathelicidin antimicrobial protein: CAMP); |
| M 2.3 | 94 | Erythrocytes, Red, | Erythrocytes. Includes hemoglobin genes |
| | | Anemia, Globin, | (HGBs) and other erythrocyte-associated genes |
| | | Hemoglobin | (erythrocytic alkirin: ANK1, Glycophorin C: |
| | | | GYPC, hydroxymethylbilane synthase: HMBS, |
| | | | erythroid associated factor: ERAF); |
| M 2.4 | 118 | Ribonucleoprotein, | Ribosomal proteins. Including genes encoding |
| | | 60S, nucleolus, | ribosomal proteins (RPLs, RPSs), Eukaryotic |
| | | Assembly, | Translation Elongation factor family members |
| | | Elongation | (EEFs) and Nucleolar proteins (NPM1, NOAL2, |
| | | | NAP1L1); |
| M 2.5 | 242 | Adenoma, Interstitial, | Undetermined. This module includes genes |
| | | Mesenchyme, | encoding immune-related (CD40, CD80, |
| | | Dendrite, Motor | CXCL12, IFNA5, IL4R) as well as cytoskeleton- |
| | | | related molecules (Myosin, Dedicator of |
| | | | Cytokenesis, Syndecan 2, Plexin C1, |
| | | | Distrobrevin); |
| M 2.6 | 110 | Granulocytes, | Myeloid lineage. Related to M 1.5. Includes |
| | | Monocytes, Myeloid, | genes expressed in myeloid lineage cells |
| | | ERK, Necrosis | (IGTB2/CD18, Lymphotoxin beta receptor, |
| | | | Myeloid related proteins 8/14 Formyl peptide |
| | | | receptor 1), such as Monocytes and Neutrophils; |
| M 2.7 | 43 | No keywords | Undetermined. This module is largely |
| | | extracted. | composed of transcripts with no known function. |
| | | | Only 20 genes associated with literature, |
| | | | including a member of the chemokine-like factor |
| | | | superfamily (CKLFSF8); |
| M 2.8 | 104 | Lymphoma, T-cell, | T-cells. Includes T-cell surface markers (CD5, |
| | | CD4, CD8, TCR, | CD6, CD7, CD26, CD28, CD96) and molecules |
| | | Thymus, Lymphoid, | expressed by lymphoid lineage cells |
| | | IL2 | (lymphotoxin beta, IL2-inducible T-cell kinase, |
| | | | TCF7, T-cell differentiation protein mal, |
| | | | GATA3, STAT5B); |
| M 2.9 | 122 | ERK, | Undetermined. Includes genes encoding |
| | | Transactivation, | molecules that associate to the cytoskeleton |
| | | Cytoskeletal, MAPK, | (Actin related protein 2/3, MAPK1, MAP3K1, |
| | | JNK | RAB5A). Also present are T-cell expressed |
| | | | genes (FAS, ITGA4/CD49D, ZNF1A1); |
| M 2.10 | 44 | Myeloid, | Undetermined. Includes genes encoding for |
| | | Macrophage, | Immune-related cell surface molecules (CD36, |
| | | Dendritic, | CD86, LILRB), cytokines (IL15) and molecules |
| | | Inflammatory, | involved in signaling pathways (FYB, TICAM2- |
| | | Interleukin | Toll-like receptor pathway); |
| M 2.11 | 77 | Replication, Repress, | Undetermined. Includes kinases (UHMK1, |
| | | RAS, | CSNK1G1, CDK6, WNK1, TAOK1, CALM2, |
| | | Autophosphorylation, | PRKCI, ITPKB, SRPK2, STK17B, DYRK2, |
| | | Oncogenic | PIK3R1, STK4, CLK4, PKN2) and RAS family |
| | | | members (G3BP, RAB14, RASA2, RAP2A, |
| | | | KRAS); |
| M 3.1 | 80 | ISRE, Influenza, | Interferon-inducible. This set includes |
| | | Antiviral, IFN- | interferon-inducible genes: antiviral molecules |
| | | gamma, IFN-alpha, | (OAS1/2/3/L, GBP1, G1P2, EIF2AK2/PKR, |
| | | Interferon | MX1, PML), chemokines (CXCL10/IP-10), |
| | | | signaling molecules (STAT1, STAt2, IRF7, |
| | | | ISGF3G); |
| M 3.2 | 230 | TGF-beta, TNF, | Inflammation I. Includes genes encoding |
| | | Inflammatory, | molecules involved in inflammatory processes |
| | | Apoptotic, | (e.g. IL8, ICAM1, C5R1, CD44, PLAUR, IL1A, |
| | | Lipopolysaccharide | CXCL16), and regulators of apoptosis (MCL1, |
| | | | FOXO3A, RARA, BCL3/6/2A1, GADD45B); |
| M 3.3 | 230 | Granulocyte, | Inflammation II. Includes molecules inducing |
| | | Inflammatory, | or inducible by Granulocyte-Macrophage CSF |
| | | Defense, Oxidize, | (SPI1, IL18, ALOX5, ANPEP), as well as |
| | | Lysosomal | lysosomal enzymes (PPT1, CTSB/S, CES1, |
| | | | NEU1, ASAH1, LAMP2, CAST); |
| M 3.4 | 323 | No keyword | Undetermined. Includes protein phosphates |
| | | extracted | (PPP1R12A, PTPRC, PPP1CB, PPM1B) and |
| | | | phosphoinositide 3-kinase (PI3K) family |
| | | | members (PIK3CA, PIK32A, PIP5K3); |
| M 3.5 | 19 | No keyword | Undetermined. Composed of only a small |
| | | extracted | number of transcripts. Includes hemoglobin |
| | | | genes (HBA1, HBA2, HBB); |
| M 3.6 | 233 | Complement, Host, | Undetermined. This very large set includes T- |
| | | Oxidative, | cell surface markers (CD101, CD102, CD103) as |
| | | Cytoskeletal, T-cell | well as molecules ubiquitously expressed among |
| | | | blood leukocytes (CXRCR1: fraktalkine |
| | | | receptor, CD47, P-selectin ligand); |
| M 3.7 | 80 | Spliceosome, | Undetermined. Includes genes encoding |
| | | Methylation, | proteasome subunits (PSMA2/5, PSMB5/8); |
| | | Ubiquitin, Beta- | ubiquitin protein ligases HIP2, STUB1, as well |
| | | catenin | as components of ubiqutin ligase complexes |
| | | | (SUGT1); |
| M 3.8 | 182 | CDC, TCR, CREB, | Undetermined. Includes genes encoding for |
| | | Glycosylase | several enzymes: aminomethyltransferase, |
| | | | arginyltransferase, asparagines synthetase, |
| | | | diacylglycerol kinase, inositol phosphatases, |
| | | | methyltransferases, helicases; and |
| M 3.9 | 261 | Chromatin, | Undetermined. Includes genes encoding for |
| | | Checkpoint, | protein kinases (PRKPIR, PRKDC, PRKCI) and |
| | | Replication, | phosphatases (e.g. PTPLB, PPP1R8/2CB). Also |
| | | Transactivation | includes RAS oncogene family members and the |
| | | | NK cell receptor 2B4 (CD244); |

wherein probes that bind specifically to one or more of the genes are selected from within the three or more modules and are indicative of systemic lupus erythematosus.

22. A method for selecting patients for a clinical trial comprising the steps of:
obtaining the transcriptome of a prospective patient;
comparing the transcriptome to one or more transcriptional modules that are indicative of a disease or condition that is to be treated in the clinical trial; and
determining the likelihood that a patient is a good candidate for the clinical trial based on the presence, absence or level of one or more genes that are expressed in the patient's transcriptome within one or more transcriptional modules that are correlated with success in a clinical trial.

23. The method of claim 22, wherein each module comprises a vector that correlates with a sum of the proportion of transcripts in a sample.

24. The method of claim 22, wherein each module comprises a vector and wherein one or more diseases or conditions are associated with the one or more vectors.

25. The method of claim 22, wherein each module comprises a vector that correlates to the expression level of one or more genes within each module.

26. The method of claim 22, wherein each module comprises a vector and wherein the modules selected are:
Transcriptional modules
one or more MHC/Ribosomal genes comprising MHC class I molecules: HLA-A,B,C,G,E)+Beta 2-microglobulin (B2M), Ribosomal proteins: RPLs, RPSs;
one or more Neutrophil genes comprising Lactotransferrin: LTF, defensin: DEAF1, Bacterial Permeability Increasing protein (BPI), Cathelicidin antimicrobial protein (CAMP);
one or more Ribosomal protein genes comprising RPLs, RPSs, Eukaryotic Translation Elongation factor family members (EEFs), Nucleolar proteins: NPM1, NOAL2, NAP1L1;
one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B;
one or more interferon-inducible genes comprising antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G);
and combinations thereof, wherein the transcriptional module is used to differentiate patients with SLE from other patients.

27. An array of nucleic acid probes immobilized on a solid support comprising sufficient probes from one or more modules to provide a sufficient proportion of differentially expressed genes to distinguish between one or more diseases, the probes being selected from Table 4.

28. A prognostic gene array comprising:
a customized gene array that comprises a combination of probes that are prognostic of SLE and the probes are selected from:
Transcriptional modules
one or more MHC/Ribosomal genes comprising MHC class I molecules: HLA-A,B,C,G,E)+Beta 2-microglobulin (B2M), Ribosomal proteins: RPLs, RPSs;
one or more Neutrophil genes comprising Lactotransferrin: LTF, defensin: DEAF1, Bacterial Permeability Increasing protein (BPI), Cathelicidin antimicrobial protein (CAMP);
one or more Ribosomal protein genes comprising RPLs, RPSs, Eukaryotic Translation Elongation factor family members (EEFs), Nucleolar proteins: NPM1, NOAL2, NAP1L1;
one or more T-cell surface marker genes comprising CD5, CD6, CD7, CD26, CD28, CD96, lymphotoxin beta, IL2-inducible T-cell kinase, TCF7, T-cell differentiation protein mal, GATA3, and STAT5B; and
one or more interferon-inducible genes comprising antiviral molecules (OAS1/2/3/L, GBP1, G1P2, EIF2AK2/PKR, MX1, PML), chemokines (CXCL10/IP-10), signaling molecules (STAT1, STAt2, IRF7, ISGF3G).
